(19) United States
(12) Patent Application Publication
(10) Pub. No.: US 2008/0050727 A1
(43) Pub. Date: Feb. 28, 2008

(54) Method of Genotyping Blood Cell Antigens and Kit Suitable for Genotyping Blood Cell Antigens

(76) Inventors: Sigrid Herma W. Beiboer, Hoogvliet (NL); Hendrika Wieringa-Jelsma, Almere (NL); Johannes Theodorus Den Dunnen, Rotterdam (NL); Maschenka De Haas, Koog Aan De Zaan (NL)

Correspondence Address: MORRISON & FOERSTER LLP, 12531 HIGH BLUFF DRIVE, SUITE 100, SAN DIEGO, CA, 92130-2040, US

(21) Appl. No.: 11/547,018

(22) PCT Filed: Mar. 31, 2005
(86) PCT No.: PCT/NL05/00236

§ 371 Date: Jul. 18, 2007

(30) Foreign Application Priority Data
Apr. 1, 2004 (EP) 04076046.4

(51) Int. Cl. C12Q 1/68

(52) U.S. Cl. 435/6

(57) ABSTRACT

A method of genotyping blood cell antigens comprising subjecting DNA from an individual of a mammalian species to a multiplex Polymerase Chain Reaction (PCR) to amplify and detectably label a region of the locus of at least two different blood cell antigens which contains the site of nucleotide polymorphism of said blood cell antigen and using the thus amplified and labeled DNA fragments to determine the genotype for each of said blood cell antigens. The multiplex PCR -comprises the use of at least one pair of blood cell antigen-specific chimeric primers for each blood cell antigen to be genotyped and at least one detectably labeled universal primer, preferably a pair of detectably labeled universal primers. The universal primer(s) have a unique sequence not occurring in the DNA of said mammalian species. Each chimeric primer pair comprises a left chimeric primer and a right chimeric primer, Each of them comprising a blood cell antigen-specific part at the 3′ end and a universal part at the 5′ end. The base sequence of the universal part of the chimeric primers corresponds to the base sequence of said at least one universal primer. The blood cell antigen-specific parts of the chimeric primer pair enclose a region of the locus of the blood cell antigen which contains the site of nucleotide polymorphism of said blood cell antigen. A kit for genotyping blood cell antigens by this method. A set of blood cell antigen-specific chimeric primer pairs and a set of blood cell antigen allele-specific oligonucleotide probes.

### FIELD OF THE INVENTION

This invention is in the field of genotyping of blood cell antigens, more particularly antigens on red blood cells (blood group antigens), blood platelets (platelet antigens) and leukocytes (leukocyte antigens).

The present invention provides a method of genotyping blood cell antigens and a kit suitable for genotyping blood cell antigens, and provides sets of primers and probes useful for genotyping blood cell antigens.

### BACKGROUND OF THE INVENTION

When blood, or a blood fraction, such as red blood cells (erythrocytes or red cells), platelets (thrombocytes) and white blood cells (leukocytes), derived from a donor are administered to another person (or more generally to another mammalian individual), serious adverse reactions may occur when the donor blood or blood fraction does not match properly with the blood of the recipient. Well known are transfusion reactions (agglutination) occurring when for example blood from a donor of blood group A is given to a person of blood group B (blood group antigens A and B belong to the AB0 system). When blood of a rhesus D (RhD) positive donor is given to a RhD negative patient there is a high chance that alloantibody formation occurs. RhD antibodies will lead to rapid destruction of RhD-positive red cells and to transfusion reactions. Furthermore, when a woman with red cell or platelet antibodies becomes pregnant, those antibodies can cross the placenta and can destruct the red cells or the platelets of the unborn child. This can lead to severe hemolysis resulting in anaemia, jaundice (after birth) and if not treated it can be fatal or lead to cerebral damage. Thus, to avoid transfusion reactions, the current blood transfusion policy is to transfuse only AB0 and RhD matched red cells. To avoid alloantibody formation with possible complications during pregnancy for women in childbearing age only AB0, RhD and K1 matched red cells are transfused. Possibly, in the future, also Rhc and RhE matched red cells will be given to women in childbearing age.

Various other blood group antigens (red cell antigens) exist, however, and these may also cause serious problems when non-matching donor blood is given to a recipient with alloantibodies. Typing of platelet-specific antigens is important for the diagnosis and therapy of the patients since different alloimmune thrombocytopenic syndromes can occur. If a woman has developed anti-platelet antigen antibodies (in most cases Human Platelet Antigen (HPA) type 1a antibodies) and mostly developed during a pregnancy, these antibodies can lead to fetal platelet destruction with an increased bleeding tendency in the unborn. In a number of cases this will lead to intracranial bleeding. To prevent bleeding, HPA-1a-negative platelets need to be transfused.

Platelets are usually transfused without previous typing of platelet antigens [human platelet antigens (HPA) in humans] of the donor and the recipient. Transfusions of non-matching blood or blood fractions may cause the generation of alloantibodies, especially in patients who need frequent or recurrent red cell or platelet (or leukocyte) transfusions. If multiple alloantibodies have formed, or if the alloantibodies are directed against high-frequency antigens, it can be a problem to find compatible red cells or platelets. According to a published study (Seltsam et al., 2003), transfusion support was unsatisfactory in about one-third of the hospitalized patients with antibodies to high-frequency antigens.

The classical method of testing for blood group antigens and antibodies is phenotyping by the hemagglutination test. The technique of this serological test is simple and inexpensive, but its costs and difficulties increase when multiple assays need to be done for complete typing and it requires availability of a large number of specific antisera. In The Netherlands alone, the total number of blood donors is in the order of magnitude of 500,000 people and it is estimated that the number of donors increases each year with about 60,000 new donors. For red cells, there are at least 29 blood group antigen systems (each with a number of different alleles). Furthermore, it will be relevant to type for high-frequency antigens and low-frequency antigens. The number of clinically relevant blood cell antigen systems is about 60. Complete phenotyping of all blood donors is therefore expensive, laborious, time consuming and not feasible due to lack of sufficient typing reagents.

The molecular basis for most blood cell antigen systems is known. Most blood group antigens, platelet antigens and neutrophil antigens are bi-allelic and are the result of a single nucleotide polymorphism (SNP). These SNPs may be used for genotyping. Innumerable DNA-based assays have been described in the scientific literature for the genotyping of blood groups and platelet antigens. These include PCR-RFLP, allele-specific PCR, sequence specific PCR as single or multiplex assays, real-time quantitative PCR, a single-nucleotide dye terminator extension method and high-throughput bead technology (reviewed by Reid, 2003). Semiautomated methods using a mass spectrophotometer or pyrosequencer may also be used for genotyping.

For example, Randen et al. (2003) recently disclosed a genotyping of the human platelet antigens 1, 2, 3, 4, 5 and Gov (recently called HPA-15, Metcalfe et al., 2003) by melting curve analysis using LightCycler technology. The biallelic systems HPA-1 through 5 and Gov are the ones most frequently involved in disease (Berry et al., 2000), making them important targets for genotyping. The LightCycler technology involves an amplification of relevant fragments of donor DNA by PCR using a specific primer pair for each of the above mentioned platelet antigens. By using fluorescent hybridization probes and melting curve analysis it was possible to achieve a simultaneous detection of both alleles of a platelet antigen in the same capillary, without a need of the laborious and time consuming gel electrophoresis of earlier genotyping methodology.

However, as with all other described blood cell antigen genotyping methods, also the LightCycler technology is not capable of performing the enormous task of a complete genotyping of blood donors, which would require methodology which is suitable for high-throughput screening. Typing each year 60,000 donors after two different donations for 60 blood group and platelet antigens would involve more than 7 million typing tests per year or about 140,000 typing tests per week. A suitable high-throughput method, which is rapid and reliable, is highly desirable for this task.

Multiplex Polymerase Chain Reaction

The technique of Polymerase Chain Reaction (PCR) has developed much since it was introduced in the 1980's. Chamberlain et al. (1988) taught multiplex PCR as a general technique for the amplification of multiple loci in (genomic) DNA. Herein, instead of one primer pair for the amplification of one locus, more primer pairs are added in one reaction mixture. However, the development of such multiplex PCRs is limited by the complexity of the amplification reaction. Reaction components and cycling conditions must be adjusted for each extra pair of primers. Shuber et al. (1995) introduced ‘chimeric’ sequence-specific primers to circumvent this problem. These primers are complementary to the template DNA and contain an unrelated 20-nucleotide tag at the 5′ end (universal sequence). Although the primers were designed in such a way that their predicted melting temperatures are similar to those of the other primers and have a calculated AG for primer duplexing below −10 kcal/mole, the concentration of the primers still needs to be adjusted to obtain similar yields of PCR product. To circumvent this problem and to also reduce primer-dimer formation further, Belgrader et al. (1996) applied only a limited amount of chimeric primer (2 pmol) and added an overload of universal primer after 15 PCR cycles. Thermal cycling proceeded then for another 25 cycles at a lower annealing temperature. Brownie et al. (1997) included the universal primer already in the PCR reaction mixture at the start. After 4 cycles of PCR the annealing temperature is raised from 60° C. to 74° C. and another 35 cycles of PCR is performed. Further, a nested PCR is taught by Heath et al. (2000) who applies in the second PCR two (complementary) universal primers, one of them carrying a fluorescent tag attached to the 5′ end.

So far, multiplex PCR has only been used with relatively small sets of primer pairs for allele-specific amplification. Neither the feasibility of using a multiplex PCR in a method of genotyping a large number of blood cell antigens, nor the nature of the PCR conditions and the primer mixtures that are suitable for such blood cell antigen genotyping, nor a practical, rapid and reliable method for analyzing the products of the amplification to assign the clinically relevant blood cell antigen genotypes have been described in the prior art.

DNA Microarrays

Several approaches using microarray technology are known in the general field of genotyping. One of these approaches is the so-called mini-sequencing method, which includes an allele-specific extension either on the microarray or in solution (Pastinen et al., 1997; Fan et al., 2000). A difficulty in this approach is the occurrence of non-specific primer extension and further optimization of primers or the extension method itself is necessary (Pastinen et al., 2000; Lindroos et al., 2002). Moreover, this method requires laborious steps of enzymatic treatment and purification.

Another approach is called allele-specific oligonucleotide hybridization (ASO) on microarrays. This approach relies on the thermal stability of the target and short oligonucleotide probes for genotype determination (Hacia et al., 1996; Wang et al., 1998). The first light-generated oligonucleotide arrays were developed in 1991 (Fodor et al.). The method has been used for the determination of new SNPs or for resequencing. It has also been used for genotyping (Evans et al., 2002) by spotting the PCR amplification products derived from patient samples into an array and contacting the array with allele-specific oligos to discriminate the alleles of the patient. Many variations have been described, using different kinds of tools, like enzymes, nanoparticle probes, artificial nucleotides, thermal gradients, flow-through arrays, blocking oligonucleotides, for improving the sensitivity or specificity (Lu et al., 2002; Park et al., 2002; Prix et al., 2002; Kajiyama et al., 2003; Jobs et al., 2003; Van Beuningen et al., 2001; Iwasaki et al., 2002). Wen et al., 2000, made a comparison of the sensitivity and specificity of an oligonucleotide array analysis of TP53 mutations with conventional DNA sequence analysis. The oligonucleotide array used contained a plurality of probes per SNP. To immobilize the oligos to the matrix support, usually a glass slide, the probes may be provided with a spacer and an amine group (Guo et al., 2001; Wen et al., 2003). Various microarray formats have been described, for example slides with a 96-microarray format containing 250 spots per array to improve throughput (Huang et al., 2001). Flow-through systems have been described in order to reduce the hybridization time and thereby increase throughput (Cheek et al., 2001; Van Beuningen et al., 2001).

So far, DNA microarray methods have not been applied to genotyping of blood cell antigens and neither the feasibility of using a DNA microarray in a method of genotyping a large number of blood cell antigens, nor the nature of the oligonucleotide probes and microarray formats that are suitable for such blood cell antigen genotyping, nor a practical, rapid and reliable method for analyzing the hybridization results to assign the clinically relevant blood cell antigen genotypes have been described in the prior art.

### SUMMARY OF THE INVENTION

An object of the present invention is to provide methods and means allowing a practical, rapid and reliable genotyping of a large number of blood cell antigens.

Another object of the invention is to develop a high-throughput technique which allows genotyping of the whole existing donor cohort and/or the donor cohort increase for a number of blood cell antigens in the order of magnitude of 20 and ultimately even some 60 blood cell antigen systems, to thereby facilitate the selection of correct donor blood and improve the safety of blood transfusion.

Again another object of the invention is to achieve an essentially complete and reliable genotyping, at least covering the majority of clinically relevant blood cell antigen systems, using simple apparatus, in a short time, such as less than 30 hours, or less than 24 hours, preferably less than 6 hours and more preferably less than 2 hours or even less than 1 hour, on a single DNA sample subjected to one PCR reaction in a single reaction tube to simultaneously amplify and detectably label relevant DNA fragments.

These objects are achieved by the present invention which provides, in one aspect, a method of genotyping blood cell antigens comprising subjecting DNA from an individual of a mammalian species to a multiplex Polymerase Chain Reaction (PCR) to amplify and detectably label a region of the locus of at least two different blood cell antigens which contains the site of nucleotide polymorphism of said blood cell antigen and using the thus amplified and labeled DNA fragments to determine the genotype for each of said blood cell antigens, said multiplex PCR comprising the use of at least one pair of blood cell antigen-specific chimeric primers for each blood cell antigen to be genotyped and at least one detectably labeled universal primer, wherein said at least one universal primer has a unique sequence not occurring in the DNA of said mammalian species, and wherein each chimeric primer pair comprises a left chimeric primer and a right chimeric primer, each of them comprising a blood cell antigen-specific part at the 3′ end and a universal part at the 5′ end, wherein the base sequence of the universal part of the chimeric primers corresponds to the base sequence of said at least one universal primer, and wherein said blood cell antigen-specific parts of the chimeric primer pair enclose a region of the locus of the blood cell antigen which contains the site of nucleotide polymorphism of said blood cell antigen.

In the method of the invention, it is preferred that a pair of detectably labeled universal primers with a unique sequence not occurring in the DNA of said mammalian species is used and that for each chimeric primer pair the base sequence of the universal part of one member of the chimeric primer pair corresponds to the base sequence of one member of the universal primer pair and the base sequence of the universal part of the other member of the chimeric primer pair corresponds to the base sequence of the other member of the universal primer pair.

Further, it is strongly preferred in the present invention that the genotype for each of said blood cell antigens is determined by hybridizing the products of the multiplex PCR amplification, after denaturation, to blood cell antigen allele-specific oligonucleotide probes contained in a DNA array or presented in any other form, such as supported on beads, and analyzing the hybridization pattern.

In another aspect, the present invention provides a kit for genotyping blood cell antigens by a method as defined herein, comprising at least one pair of blood cell antigen-specific chimeric primers for each blood cell antigen to be genotyped and one pair of detectably labeled universal primers, both as defined herein.

This invention further provides a set of blood cell antigen-specific chimeric primer pairs useful in a multiplex PCR, and a set of blood cell antigen allele-specific oligonucleotide probes useful for genotyping blood cell antigens.

### BRIEF DESCRIPTION OF THE DRAWING

FIG. 1 depicts the results of a multiplex PCR of 19 blood group antigens with or without the presence of universal MAPH primers. Very little amount of PCR product is amplified during the first amplification cycles (lane 2), but enough to be used as template by the universal MAPH primers in the following cycles. The MAPH primers perform the actual amplification (lane 3), so adjustments of primer concentrations are hardly necessary and similar yields of PCR product are obtained.

FIG. 2 depicts the pattern obtained with ABI capillary sequencer from the multiplex PCR of 19 blood group antigens as described in this invention.

FIG. 3 depicts the scan results of the hybridisation of 6 donor samples, each comprising the 6 human platelet antigen 1 through 5 and Gov PCR products. The PCR products of one sample were hybridised to two arrays, that is 4 blocks. The original scans have red spots and a black background. For better visualisation this is changed to grayscale and inverted.

### DETAILED DESCRIPTION OF THE INVENTION

The invention provides a method of genotyping blood cell antigens comprising subjecting DNA from an individual of a mammalian species to a multiplex Polymerase Chain Reaction (PCR) to amplify and detectably label a region of the locus of at least two different blood cell antigens which contains the site of nucleotide polymorphism of said blood cell antigen and using the thus amplified and labeled DNA fragments to determine the genotype for each of said blood cell antigens, said multiplex PCR comprising the use of at least one pair of blood cell antigen-specific chimeric primers for each blood cell antigen to be genotyped and at least one detectably labeled universal primer, wherein said at least one universal primer has a unique sequence not occurring in the DNA of said mammalian species, and wherein each chimeric primer pair comprises a left chimeric primer and a right chimeric primer, each of them comprising a blood cell antigen-specific part at the 3′ end and a universal part at the 5′ end, wherein the base sequence of the universal part of the chimeric primers corresponds to the base sequence of said at least one universal primer, and wherein said blood cell antigen-specific parts of the chimeric primer pair enclose a region of the locus of the blood cell antigen which contains the site of nucleotide polymorphism of said blood cell antigen.

More particularly, the invention provides a method of genotyping blood cell antigens comprising subjecting DNA from an individual of a mammalian species to a multiplex Polymerase Chain Reaction (PCR) to amplify and detectably label a region of the locus of at least two different blood cell antigens which contains the site of nucleotide polymorphism of said blood cell antigen and using the thus amplified and labeled DNA fragments to determine the genotype for each of said blood cell antigens, said multiplex PCR comprising the use of at least one pair of blood cell antigen-specific chimeric primers for each blood cell antigen to be genotyped and one pair of detectably labeled universal primers, wherein said pair of universal primers comprises a forward universal primer and a reverse universal primer each having a unique sequence not occurring in the DNA of said mammalian species, and wherein each chimeric primer pair comprises a left chimeric primer and a right chimeric primer, each of them comprising a blood cell antigen-specific part at the 3′ end and a universal part at the 5′ end, wherein the base sequence of the universal part of one of these chimeric primers corresponds to the base sequence of one of the universal primers and the base sequence of the universal part of the other chimeric primer corresponds to the base sequence of the other of the universal primers, and wherein said blood cell antigen-specific parts of the chimeric primer pair enclose a region of the locus of the blood cell antigen which contains the site of nucleotide polymorphism of said blood cell antigen.

The invention involves a particular sequence of steps that in combination allows to achieve the above mentioned objects. This sequence of steps comprises isolation of DNA from a sample which is to be subjected to the genotyping analysis of this invention, followed by one of the key aspects of the present invention, a multiplex PCR of gene fragments carrying the single nucleotide polymorphisms (SNPs) that are responsible for blood cell antigen differences. This multiplex PCR applies a mixture of different primers, including two or more pairs of chimeric primers and in addition including a labelled universal primer or a pair of labelled universal primers. Then the thus amplified and labelled DNA fragments are denatured (such as by heating) and are contacted with a set of probes, preferably contained in a microarray comprising a multitude of allele-specific probes, under conditions which support hybridisation between matching sequences. In the next step, the presence of labelled fragments is measured for each spot in the microarray, and finally the signals measured are interpreted to type the sample. The probes may be presented also in other formats, for example as probes supported on beads (glass beads, polystyrene beads, etc.), or probes attached to the wall of a suitable container, etc. An array format is however the most preferred embodiment.

Species

The invention allows the genotyping of blood cell antigens not only of humans, but of all animals with a blood cell antigen system. For all practical purposes, genotyping will relate to blood cell antigens of mamnmals, including for example farm animals and pets, wild-life animals and generally all animals of economic, emotional or wild life preservation value. Most preferably, however, the invention concerns the genotyping of human blood cell antigens.

Blood Cell Antigens

The blood cell antigens that may be genotyped by the present invention include typical blood group antigens, such as Kidd, Duffy, Kell and Rhesus. The blood group antigens of the AB0 system may be genotyped by the present invention as well, although it may be necessary, because of their extreme importance, to perform the existing and well-accepted serological test. Other blood group antigens that may be genotyped by the present invention comprise antigen variants and or gene variants leading to gene silencing of the blood group systems MNSs, Rhesus (Rh), Kell, Kidd, Duffy, Colton, Diego, Dombrock, Lutheran, Lewis, Cartwright, Landsteiner Wiener (LW), Cromer, Knops, Kx, Indian, Gerbich, Hh, Chido/Rodgers, GIL, I, JMH, OK, P-related, RAPH-MER, Scianna, Xg, YT, etc.

In addition to the blood group antigens, also platelet and leukocyte (especially neutrophil) antigens may be genotyped by the method of this invention. The clinically most relevant platelet antigens, at least in the Western world, comprise HPA-1, HPA-2, HPA-3, HPA-4, HPA-5 and Gov (or HPA-15). Other platelet antigens that may be genotyped by the present invention, even though they are considered of lesser importance, comprise for example human platelet antigens 6 through 14 and 16. Examples of typical Human Neutrophil Antigens that may be genotyped by the method of the invention include HNA-1, 4 and 5. Others, such as HNA-2 and 3, can be genotyped as soon as their molecular basis has been elucidated.

It is conceived that the present invention is useful even when only a restricted number of blood cell antigens is genotyped. It may for some purposes be sufficient that only the red cell antigens are genotyped, or only the platelet antigens, or only a selected group of antigens which is relevant in a certain context. The minimum number of blood cell antigens to be genotyped by the present invention is two blood cell antigens, for example the blood cell antigen RhD and the RHD gene variant RhDΨ, or the blood cell antigen JK1/2 (Jk^{a}/Jk^{b}) and the blood cell antigen FY1/2 (Fy^{a}/Fy^{b}). Genotyping of a larger number of blood cell antigens is clearly preferred, for example three, four, five, and especially six or more. For most purposes, however, a more or less complete genotyping, at least covering most of the clinically relevant blood cell antigens, is preferred. Most preferred, the invention includes at least six different chimeric primer pairs specific for HPA's 1 through 5 and HPA-15, and at least 10 or 11 or 12 chimeric primer pairs specific for red cell antigens, such as the various Kidd, Duffy, Kell and Rhesus antigens.

The DNA

The DNA subjected to the multiplex PCR will usually be genomic DNA of the individual whose blood cell antigens are to be genotyped The donor DNA may be obtained from any suitable source, for example from EDTA, heparin or citrate treated blood samples or buffy coats, using isolation procedures and means well known to the skilled person, such as for example the commercially available Qiagen Blood DNA extraction kit, use of a salting-out method, such as by the standard protocol of Miller et al. (1988), or use of the Roche Magnapure.

Universal Primers

The method of the invention uses at least one universal primer carrying a detectable label, preferably a pair of universal primers both carrying a detectable label. The sequence of the universal primer(s) corresponds with the sequence of the universal part at the 5′ end of the chimeric primers. It is possible to use only one universal primer. In that case, all chimeric primers have an identical base sequence at their 5′ end, i.e. the universal part at the 5′ end of the chimeric primers corresponds with the sequence of said single universal primer. Practically, however, it was found preferable to use two different universal primers (i.e. a pair of universal primers). In that case, each pair of chimeric primers contains a primer with a universal part corresponding to one of the universal primers and a primer with a universal part corresponding to the other universal primer.

It is important that the universal primer(s) do not hybridize to the DNA which is to be genotyped. Assuming that the DNA to be genotyped is human genomic DNA, the universal primer(s) (and the corresponding parts of the chimeric primers) should not hybridize with human genomic DNA and therefore have a unique sequence not occurring in said DNA. Preferably, the sequence of the universal primer(s) differs significantly from any sequence occurring in the human genome. Further, it is strongly preferred that the universal primer(s) are designed such that there Tm value is similar to the Tm value of the blood cell antigen specific part of the chimeric primers. Preferably, the Tm value of the universal primer(s) is between 50 and 70° C., more preferably between 56 and 68° C. It is also preferred that the universal primer(s) have a length of between 12 and 30 bases, more preferably between 15 and 25 or even between 16 and 20 bases.

Very good results were obtained with universal primers disclosed in White et al., 2002, with the sequences ggccgcgggaattcgatt (SEQ ID NO:1, forward MAPH), with a Tm of 67.55° C., and gccgcgaattcactagtg (SEQ ID NO:2, reverse MAPH) with a Tm of 57.94° C.

Detectable Labels

The universal primer(s) carry a detectable label. The label may be any label suitable for the purpose, such as a radioactive atom or group, an enzyme, such as an enzyme catalyzing a measurable conversion of a substrate thereof, a dye, a fluorescent substance, a chemiluminescent substance, biotin, etc. Most preferably, the label is a fluorescent group, of which many are known to the person skilled in the art. Examples thereof are Cy3, Cy5, Fluorescein (FITC), Phycoerythrin (PE), Rhodamine, Hex, Tet, Fam, etc.

Most preferably, the invention uses the fluorescent group Cy5, which is a sulfoindocyanine dye (Mujumdar et al., 1993).

Usually, in particular when the label is a moiety attached to the oligonucleotide, it will be attached to the 5′ end of the oligonucleotide sequence of the universal primer(s).

Because the universal primer(s) are labeled, while the chimeric primers are not labelled, the invention achieves very efficient labeling simultaneous with amplification, while the various chimeric primers can be used without previous labeling.

Chimeric Primers

The chimeric primers used in this invention have a universal part at their 5′ end and a blood cell antigen-specific part at their 3′ end. The oligonucleotide sequence of the universal part corresponds with the sequence of the universal primer(s) and requires no further elucidation here.

As to the blood cell antigen-specific part of the chimeric primers, it is most preferable that these parts have similar Tm values, both in comparison with blood cell antigen-specific parts of other chimeric primers in the mixture, and in comparison with the universal primers used. Thus, it is preferred that these blood cell antigen-specific parts of the chimeric primers have Tm values between 50 and 70° C., more preferably between 55 and 65° C. and most preferably between 56 and 62° C. It is also preferred that the blood cell antigen-specific parts of the chimeric primers have a length of betveen 12 and 35 bases, more preferably between 15 and 30 or even between 16 and 25 bases.

It is further preferred that the blood cell antigen-specific parts of the chimeric primers are selected in such a way that the PCR amplification results in oligonucleotide products with a length of between 50 and 800, preferably between 80 and 500, most preferably between 100 and 400 or 300 nucleotides. A product length of from 100 to 200 nucleotides would be ideal.

Various software products are available that can be used to design suitable blood cell antigen-specific parts of the chimeric primers. On the basis of the publicly available DNA sequences of the blood cell antigen-encoding genes, the software product Primer3 (http://www.broad.mit.edu/cgi-bin/primer/primer3 www.cgi) allows to design primers. To check for any unspecific genomic DNA binding the CELERA database can be used and to check for any primer-dimer formation the Oligo6 software (Medprobe) can be used. It is preferred to select primers having a calculated ΔG below −10 kcal/mole.

A list of preferred chimeric primers includes the following:

| | | |
|---|---|---|
| HPA1-left | gccgcgaattcactagtgcttcaggtcacagcgaggt | SEQ ID NO:3 |
| HPA1-right | ggccgcgggaattcgattgctccaatgtacggggtaaa | SEQ ID NO:4 |
| HPA2-left | gccgcgaattcactagtgtgaaaggcaatgagctgaag | SEQ ID NO:5 |
| HPA2-right | ggccgcgggaattcgattagccagactgagcttctcca | SEQ ID NO:6 |
| HPA3-left | gccgcgaattcactagtggcctgaccactcctttgc | SEQ ID NO:7 |
| HPA3-right | ggccgcgggaattcgattggaagatctgtctgcgatcc | SEQ ID NO:8 |
| HPA4-left | gccgcgaattcactagtgatccgcaggttactggtgag | SEQ ID NO:9 |
| HPA4-right | ggccgcgggaattcgattccatgaaggatgatctgtgg | SEQ ID NO:10 |
| HPA5-left | gccgcgaattcactagtgtccaaatgcaagttaaattaccag | SEQ ID NO:11 |
| HPA5-right | ggccgcgggaattcgattacagacgtgctcttggtaggt | SEQ ID NO:12 |
| HPA15-left | gccgcgaattcactagtgtgtatcagttcttggttttgtgatg | SEQ ID NO:13 |
| HPA15-right | ggccgcgggaattcgattaaaaccagtagccacccaag | SEQ ID NO:14 |
| JK1/2-left | gccgcgaattcactagtggtctttcagccccatttgag | SEQ ID NO:15 |
| JK1/2-right | ggccgcgggaattcgattgttgaaaccccagagtccaa | SEQ ID NO:16 |
| FY1/2-left | gccgcgaattcactagtggaattcttcctatggtgtgaatga | SEQ ID NO:17 |
| FY1/2-right | ggccgcgggaattcgattaagaagggcagtgcagagtc | SEQ ID NO:18 |
| GATAbox-left | gccgcgaattcactagtgggccctcattagtccttgg | SEQ ID NO:19 |
| GATAbox-right | ggccgcgggaattcgattgaaatgaggggcatagggata | SEQ ID NO:20 |
| Fyx-left | gccgcgaattcactagtgtcatgcttttcagacctctcttc | SEQ ID NO:175 |
| Fyx-right | ggccgcgggaattcgattcaagacgggcaccacaat | SEQ ID NO:176 |
| KEL1/2-left | gccgcgaattcactagtgaagggaaatggccatactga | SEQ ID NO:21 |
| KEL1/2-right | ggccgcgggaattcgattagctgtgtaagagccgatcc | SEQ ID NO:22 |
| KEL3/4-left | gccgcgaattcactagtggcctcagaaactggaacagc | SEQ ID NO:23 |
| KEL3/4-right | ggccgcgggaattcgattagcaaggtgcaagaacactct | SEQ ID NO:24 |
| KLBL6/7-left | gccgcgaattcactagtggcagcaccaaccctatgttc | SEQ ID NO:177 |
| KEL6/7-right | ggccgcgggaattcgatttcaggcacaggtgagcttc | SEQ ID NO:178 |
| RHCEex2for | gccgcgaattcactagtgcgtctgcttccccctcc | SEQ ID NO:25 |
| RHex2rev | ggccgcgggaattcgattctgaacagtgtgatgaccacc | SEQ ID NO:26 |
| RHDex3-left | gccgcgaattcactagtgtcctggctctccctctct | SEQ ID NO:179 |
| RHCEex3-right | ggccgcgggaattcgatttttttcaaaaccccggaag | SEQ ID NO:180 |
| RHCEex5-left | gccgcgaattcactagtgggatgttctggccaagtg | SEQ ID NO:27 |
| RHex5rev | ggccgcgggaattcgattggctgtcaccacactgactg | SEQ ID NO:28 |
| RHDΨ-left | ggccgcgggaattcgattgtagtgagctggcccatca | SEQ ID NO:29 |
| RHDΨ-right | gccgcgaattcactagtgtgtctagtttcttaccggcaagt | SEQ ID NO:30 |
| RHD-leftB | gccgcgaattcactagtgttataataacacttgtccacaggg | SEQ ID NO:31 |
| RHD-rightC | ggccgcgggaattcgattcggctccgacggtatc | SEQ ID NO:32 |
| BigC-left | gccgcgaattcactagtgggccaccaccatttgaa | SEQ ID NO:33 |
| BigC-rightintron2 | ggccgcgggaattcgattccatgaacatgccacttcac | SEQ ID NO:34 |
| RhDVI-left (fw) | ggccgcgggaattcgattctttgaattaagcacttcacaga | SEQ ID NO:181 |
| RhDVI-right (rev) | gccgcgaattcactagtggccagaatcacactcctgct | SEQ ID NO:182 |
| MN-left | gccgcgaattcactagtgtgagggaatttgtcttttgca | SEQ ID NO:35 |
| MN-right | ggccgcgggaattcgattcagaggcaagaattcctcca | SEQ ID NO:36 |
| Ss-left | gccgcgaattcactagtgtttttctttgcacatgtcttt | SEQ ID NO:183 |
| Ss-right | ggccgcgggaattcgatttctttgtctttacaatttcgtgtg | SEQ ID NO:184 |
| U-left | gccgcgaattcactagtgcgctgatgttatctgtcttatttttc | SEQ ID NO:37 |
| U-right | ggccgcgggaattcgattgatcgttccaataataccagcc | SEQ ID NO:38 |
| DO-left | ggccgcgggaattcgatttgatccctccctatgagctg | SEQ ID NO:185 |
| DO-right | gccgcgaattcactagtgttatatgtgctcaggttcccagt | SEQ ID NO:186 |
| JO-left | gccgcgaattcactagtgcctggcttaaccaaggaaaa | SEQ ID NO:39 |
| JO-right | ggccgcgggaattcgatttcatactgctgtggagtcctg | SEQ ID NO:40 |
| Colton-left | gccgcgaattcactagtggccacgaccctctttgtct | SEQ ID NO:187 |
| Colton-right | ggccgcgggaattcgatttacatgagggcacggaagat | SEQ ID NO:188 |
| Diego-left | gccgcgaattcactagtgacttattcacgggcatccag | SEQ ID NO:189 |
| Diego-right | ggccgcgggaattcgattaagctccacgttcctgaaga | SEQ ID NO:190 |
| Wr-left | gccgcgaattcactagtgggcttcaaggtgtccaactc | SEQ ID NO:636 |
| Wr-right | ggccgcgggaattcgattaggatgaagaccagcagagc | SEQ ID NO:637 |
| Yt-left | ggccgcgggaattcgattccttcgtgcctgtggtagat | SEQ ID NO:638 |
| Yt-right | gccgcgaattcactagtgttctgggacttctgggaatg | SEQ ID NO:639 |
| Lu-left | gccgcgaattcactagtgggacccagagagagagagactg | SEQ ID NO:640 |
| Lu-right | ggccgcgggaattcgattgggagtccagctggtatgg. | SEQ ID NO:641 |

Most preferred are the chimeric primers with SEQ ID NOS:3 to 40.

The above mentioned software products would allow the skilled person to design additional primers for other blood cell antigens. The present invention encompasses the use of a set of similar chimeric primers having the same blood cell antigen-specific part but a different universal part.

Proportions of the Primers

To achieve the aims of the invention, and in particular to avoid the occurrence of primer-dimers as much as possible, it is important that only a minimal proportion of the chimeric primers is used and that amplification is due to a large extent to elongation of the universal primers. Practically, it is preferred that the universal primers are used in a molar amount which is at least 10 times, more preferably at least 40 times the molar amount of each chimeric primer. Per amplification reaction 5 nM of each chimeric primer and 0.2 μM of each universal primer per chimeric primer pair is preferred. Per microliter reaction volume, an amount of 5 femtomol (5*10⁻¹⁵ mol) of each chimeric primer would be preferred, while about 0.2 pmol (0.2*10⁻¹² mol) of each universal primer is used per chimeric primer pair.

Multiplex PCR Conditions

The multiplex PCR in the method of this invention uses a mixture of universal and chimeric primers which are present from the beginning of the reaction. The PCR as used in this invention does not distinguish between a separate first part in which only chimeric primers are extended, and a second part in which only universal primers are extended. The present invention applies an annealing or primer extension temperature in the later cycles of the PCR which is the same as the annealing or primer extension temperature used in the first few cycles of the PCR. The inventors found that a change of reaction conditions in order to switch from chimeric primer extension to universal primer extension is not required.

The DNA polymerase used in the multiplex PCR is preferably a heat-resistant DNA polymerase, such as Taq DNA polymerase. Other heat-resistant DNA polymerases are useful as well, and even heat-sensitive DNA polymerases, although this may require repeated addition of a fresh amount of DNA polymerase. Suitable DNA polymerases are commercially available, for example as a component of multiplex PCR kits, like the Qiagen multiplex kit. Such kits also contain other necessary components, such as the required dNTP's and a suitable buffer system.

Apparatus for carrying out PCR thermocycling is commercially available as well, for example the MWG AG Biotech PrimusHT thermal cycler.

The amplification of the invention usually starts with a heat treatment to activate the DNA polymerase, for example 15 min at 95° C. Subsequently, the thermal cycling begins. Each cycle includes a heat denaturation step, an annealing step (to bind primer by hybridization to its template) and a primer extension or elongation step. In the invention, it is preferred that the heat denaturation step involves heating at a temperature of 90 to 98° C., more preferably at about 94 or 95° C., and takes a time of from 15 to 60 seconds (shorter and especially longer times are allowable), more preferably about 30 seconds. In the invention, it is further preferred that the annealing step involves heating at a temperature of 54 to 60° C., more preferably at about 57° C., and takes a time of from 60 to 120 seconds (shorter and especially longer times are allowable), more preferably about 90 seconds. In the invention, it is further preferred that the primer extension step involves heating at a temperature of 68 to 76° C., more preferably at about 72° C., and takes a time of from 60 to 120 seconds (shorter and especially longer times are allowable), more preferably about 90 seconds. A final treatment at the temperature of the primer extension step for a much longer time, for example 10 minutes, may conclude the thermal cycling.

The total number of cycles may be chosen at will, depending on the available time and the precision sought. A total of 30 cycles may be enough, but 40 or 45 or 50 cycles or more is preferable.

Genotyping

The result of the multiplex PCR is a mixture of labelled amplification products, in which ideally each product essentially comprises a region of the locus of different blood cell antigens which contains the site of nucleotide polymorphism of said blood cell antigen. In most cases of blood cell antigen polymorphisms that are presently known, the nucleotide polymorphism is a single nucleotide polymorphism (SNP), but polymorphism sites covering more than one nucleotide are encompassed as well.

To analyse the SNP's present in the mixture of labelled amplification products, various strategies are available, including sequencing of the products, but many of these are not suitable for rapid and reliable high-throughput genotyping. The present invention therefore preferably applies the special step of contacting the products of the multiplex PCR with a DNA array which contains blood cell antigen allele-specific oligonucleotide probes. Said contacting is done after denaturation, for example by heating, of the oligonucleotidic products of thie amplification and under conditions suitable for hybridization of these products to corresponding probes in the array. Purification of the products, of the amplification before contacting them with the probes is not necessary and will usually be omitted.

Probes

The probes are oligonucleotides including the site of the nucleotide polymorphism. Thereby, they are allele-specific. Although their length may vary from only 8 or 10 nucleotides up to several hundreds of nucleotides, it is preferred in this invention to use probes with a length of from 15 to 40 nucleotides, more preferably 16 or 17 to 29 or 30 nucleotides. Preferably, all probes have similar Tm values, in particular in a range of 55 to 75° C., more preferably in the range of 60 to 70° C. or especially in the range of 60 to 65° C.

Although in principle it is possible to use only one probe for each allele of each blood cell antigen, the invention applies several probes for each allele of each blood cell antigen, thereby increasing the reliability of the method. According to this invention, the array contains for each allele of each blood cell antigen at least two, preferably at least five, different sense probes and at least two, preferably at least five, antisense probes, each of them covering the site of the nucleotide polymorphism but at varying positions. Preferably, these positions are in or near the centre of the oligos.

Various software products, as mentioned above for the blood cell antigen-specific parts of the primers, are available that can be used to design suitable blood cell antigen allele-specific probes (in particular the sofware product Primer3).

A list of preferred probes includes the following (SNPs are highlighted):

| HPA1-allele a: | | |
|---|---|---|
| HPA-1aa | tacaggccctgcctctggg | SEQ ID NO:41 |
| HPA-1ab | aggccctgcctctgggct | SEQ ID NO:42 |
| HPA-1ac | ccctgcctctgggctcacc | SEQ ID NO:43 |
| HPA-1ad | tgcctctgggctcacctcg | SEQ ID NO:44 |
| HPA-1ae | ctctgggctcacctcgctg | SEQ ID NO:45 |
| HPA-1aa CR | cagcgaggtgagcccagag | SEQ ID NO:46 |
| HPA-1ab CR | cgaggtgagcccagaggca | SEQ ID NO:47 |
| HPA-1ac CR | ggtgagcccagaggcaggg | SEQ ID NO:48 |
| HPA-1ad CR | agcccagaggcagggcct | SEQ ID NO:49 |
| HPA-1ae CR | cccagaggcagggcctgta | SEQ ID NO:50 |
| HPA1-allele b: | | |
| HPA-1ba | tacaggccctgcctccggg | SEQ ID NO:51 |
| HPA-1bb | aggccctgcctccgggct | SEQ ID NO:52 |
| HPA-1bc | ccctgcctccgggctcac | SEQ ID NO:53 |
| HPA-1bd | ctgcctccgggctcacct | SEQ ID NO:54 |
| HPA-1be | ctccgggctcacctcgct | SEQ ID NO:55 |
| HPA-1ba CR | agcgaggtgagcccggag | SEQ ID NO:56 |
| HPA-1bb CR | aggtgagcccggaggcag | SEQ ID NO:57 |
| HPA-1bc CR | gtgagcccggaggcaggg | SEQ ID NO:58 |
| HPA-1bd CR | agcccggaggcagggcct | SEQ ID NO:59 |
| HPA-1be CR | cccggaggcagggcctgta | SEQ ID NO:60 |
| HPA2-allele a: | | |
| HPA-2aa | ctgacgcccacacccaag | SEQ ID NO:61 |
| HPA-2ab | ctcctgacgcccacaccc | SEQ ID NO:62 |
| HPA-2ac | ggctcctgacgcccacac | SEQ ID NO:63 |
| HPA-2ad | agggctcctgacgcccac | SEQ ID NO:64 |
| HPA-2ae | ccagggctcctgacgccc | SEQ ID NO:65 |
| HPA-2aa CR | cttgggtgtgggcgtcag | SEQ ID NO:66 |
| HPA-2ab CR | gggtgtgggcgtcaggag | SEQ ID NO:67 |
| HPA-2ac CR | gtgtgggcgtcaggagcc | SEQ ID NO:68 |
| HPA-2ad CR | gtgggcgtcaggagccct | SEQ ID NO:69 |
| HPA-2ae CR | gggcgtcaggagccctgg | SEQ ID NO:70 |
| HPA2-allele b: | | |
| HPA-2ba | cctgatgcccacacccaag | SEQ ID NO:71 |
| HPA-2bb | ctcctgatgcccacaccca | SEQ ID NO:72 |
| HPA-2bc | ggctcctgatgcccacacc | SEQ ID NO:73 |
| HPA-2bd | agggctcctgatgcccaca | SEQ ID NO:74 |
| HPA-2be | ccagggctcctgatgccc | SEQ ID NO:75 |
| HPA-2ba CR | cttgggtgtgggcatcagg | SEQ ID NO:76 |
| HPA-2bb CR | tgggtgtgggcatcaggag | SEQ ID NO:77 |
| HPA-2bc CR | ggtgtgggcatcaggagcc | SEQ ID NO:78 |
| HPA-2bd CR | tgtgggcatcaggagccct | SEQ ID NO:79 |
| HPA-2be CR | gggcatcaggagccctgg | SEQ ID NO:80 |
| HPA3-allele a: | | |
| HPA-3aa | ccatccccagcccctccc | SEQ ID NO:81 |
| HPA-3ab | gcccatccccagcccctc | SEQ ID NO:82 |
| HPA-3ac | ctgcccatccccagcccc | SEQ ID NO:83 |
| HPA-3ad1 | gctgcccatccccagccc | SEQ ID NO:84 |
| HPA-3ad | ggctgcccatccccagcc | SEQ ID NO:85 |
| HPA-3ad2 | gggctgcccatccccagc | SEQ ID NO:86 |
| HPA-3ae | ggggctgcccatcccca | SEQ ID NO:87 |
| HPA-3aa CR | gggaggggctggggatgg | SEQ ID NO:88 |
| HPA-3ab CR | gaggggctggggatgggc | SEQ ID NO:89 |
| HPA-3ac CR | ggggctggggatgggcag | SEQ ID NO:90 |
| HPA-3ad1 CR | gggctggggatgggcagc | SEQ ID NO:91 |
| HPA-3ad CR | ggctggggatgggcagcc | SEQ ID NO:92 |
| HPA-3ad2 CR | gctggggatgggcagccc | SEQ ID NO:93 |
| HPA-3ae CR | tgggatgggcagcccc | SEQ ID NO:94 |
| HPA3-allele b: | | |
| HPA-3ba | ccagccccagcccctcc | SEQ ID NO:95 |
| HPA-3bb | gcccagccccagcccct | SEQ ID NO:96 |
| HPA-3bc | ctgcccagccccagccc | SEQ ID NO:97 |
| HPA-3bd1 | gctgcccagccccagcc | SEQ ID NO:98 |
| HPA-3bd | ggctgcccagcccagc | SEQ ID NO:99 |
| HPA-3bd2 | gggctgcccagccccag | SEQ ID NO:100 |
| HPA-3be | ggggctgcccagcccca | SEQ ID NO:101 |
| HPA-3ba CR | ggaggggctggggctgg | SEQ ID NO:102 |
| HPA-3bb CR | aggggctggggctgggc | SEQ ID NO:103 |
| HPA-3bc CR | gggctggggctgggcag | SEQ ID NO:104 |
| HPA-3bd1 CR | ggctggggctgggcagc | SEQ ID NO:105 |
| HPA-3bd CR | gctggggctgggcagcc | SEQ ID NO:106 |
| HPA-3bd2 CR | ctggggctgggcagccc | SEQ ID NO:107 |
| HPA-3be CR | tggggctgggcagcccc | SEQ ID NO:108 |
| HPA4-allele a: | | |
| HPA-4aa | gccacccagatgcgaaag | SEQ ID NO:109 |
| HPA-4ab | cacccagatgcgaaagct | SEQ ID NO:110 |
| HPA-4ac | cccagatgcgaaagctca | SEQ ID NO:111 |
| HPA-4ad | cagatgcgaaagctcacc | SEQ ID NO:112 |
| HPA-4ae | gatgcgaaagctcaccag | SEQ ID NO:113 |
| HPA-4aa CR | ctggtgagctttcgcatc | SEQ ID NO:114 |
| HPA-4ab CR | ggtgagctttcgcatctg | SEQ ID NO:115 |
| HPA-4ac CR | tgagctttcgcatctggg | SEQ ID NO:116 |
| HPA-4ad CR | agctttcgcatctgggtg | SEQ ID NO:117 |
| HPA-4ae CR | ctttcgcatctgggtggc | SEQ ID NO:118 |
| HPA4-allele b: | | |
| HPA-4ba | gccacccagatgcaaaag | SEQ ID NO:119 |
| HPA-4bb | ccacccagatgcaaaagct | SEQ ID NO:120 |
| HPA-4bc | acccagatgcaaaagctcac | SEQ ID NO:121 |
| HPA-4bd | cagatgcaaaagctcacca | SEQ ID NO:122 |
| HPA-4be | gatgcaaaagctcaccagtaa | SEQ ID NO:123 |
| HPA-4ba CR | ttactggtgagcttttgcatc | SEQ ID NO:124 |
| HPA-4bb CR | tggtgagcttttgcatctg | SEQ ID NO:125 |
| HPA-4bc CR | gtgagcttttgcatctgggt | SEQ ID NO:126 |
| HPA-4bd CR | agcttttgcatctgggtgg | SEQ ID NO:127 |
| HPA-4be CR | cttttgcatctgggtggc | SEQ ID NO:128 |
| HPA5-allele a: | | |
| HPA-5aa | gagtctacctgtttactatcaaagagg | SEQ ID NO:129 |
| HPA-5ab | agtctacctgttactatcaaagaggta | SEQ ID NO:130 |
| HPA-5ac | gtctacctgtttactatcaaagaggtaa | SEQ ID NO:131 |
| HPA-5ad | ctacctgtttactatcaaagaggtaaaa | SEQ ID NO:132 |
| HPA-5ae | acctgtttactatcaaagaggtaaaaa | SEQ ID NO:133 |
| HPA-5aa CR | tttttacctctttgatagtaaacaggt | SEQ ID NO:134 |
| HPA-5ab CR | ttttacctctttgatagtaaacaggtag | SEQ ID NO:135 |
| HPA-5ac CR | ttacctctttgatagtaaacaggtagac | SEQ ID NO:136 |
| HPA-5ad CR | tacctctttgatagtaaacaggtagact | SEQ ID NO:137 |
| HPA-5ae CR | cctctttgatagtaaacaggtagactc | SEQ ID NO:138 |
| HPA5-allele b: | | |
| HPA-5ba | gagtctacctgtttactatcaaaaagg | SEQ ID NO:139 |
| HPA-5bb | agtctacctgtttactatcaaaaaggta | SEQ ID NO:140 |
| HPA-5bc | gtctacctgtttactatcaaaaaggtaa | SEQ ID NO:141 |
| HPA-5ad | ctacctgtttactatcaaaaaggtaaaa | SEQ ID NO:142 |
| HPA-5be | acctgtttactatcaaaaaggtaaaaa | SEQ ID NO:143 |
| HPA-5ba CR | tttttacctttttgatagtaaacaggt | SEQ ID NO:144 |
| HPA-5bb CR | ttttacctttttgatagtaaacaggtag | SEQ ID NO:145 |
| HPA-5bc CR | ttacctttttgatagtaaacaggtagac | SEQ ID NO:146 |
| HPA-5bd CR | tacctttttgatagtaaacaggtagact | SEQ ID NO:147 |
| HPA-5be CR | cctttttgatagtaaacaggtagactc | SEQ ID NO:148 |
| HPA15-allele a: | | |
| Gov-aa | ttattatcttgacttcagttacaggattt | SEQ ID NO:149 |
| Gov-ab | tcttgacttcagttacaggatttacc | SEQ ID NO:150 |
| Gov-ac | tgacttcagttacaggatttaccaa | SEQ ID NO:151 |
| Gov-ad | cttcagttacaggatttaccaagaat | SEQ ID NO:152 |
| Gov-ae | cagttacaggatttaccaagaatttg | SEQ ID NO:153 |
| Gov-aa CR | caaattcttggtaaatcctgtaactg | SEQ ID NO:154 |
| Gov-ab CR | attcttggtaaatcctgtaactgaag | SEQ ID NO:155 |
| Gov-ac CR | tggtaaatcctgtaactgaagtcaa | SEQ ID NO:156 |
| Gov-ad CR | ggtaaatcctgtaactgaagtcaaga | SEQ ID NO:157 |
| Gov-ae CR | aaatcctgtaactgaagtcaagataataa | SEQ ID NO:158 |
| HPA15-allele b: | | |
| Gov-ba | tatcttgacttcagttccaggatt | SEQ ID NO:159 |
| Gov-bb | cttgacttcagttccaggatttac | SEQ ID NO:160 |
| Gov-bc | gacttcagttccaggatttacca | SEQ ID NO:161 |
| Gov-bd | ttcagttccaggatttaccaag | SEQ ID NO:162 |
| Gov-be | cagttccaggatttaccaagaatt | SEQ ID NO:163 |
| Gov-ba CR | aattcttggtaaatcctggaactg | SEQ ID NO:164 |
| Gov-bb CR | cttggtaaatcctggaactgaa | SEQ ID NO:165 |
| Gov-bc CR | ggtaaatcctggaactgaagtca | SEQ ID NO:166 |
| Gov-bd CR | gtaaatcctggaactgaagtcaag | SEQ ID NO:167 |
| Gov-be CR | aatcctggaactgaagtcaagata | SEQ ID NO:168 |
| Colton-a-allele: | | |
| Co.a.1 | aacaaccagacggcggt | SEQ ID NO:191 |
| Co.a.2 | accagacggcggtccag | SEQ ID NO:192 |
| Co.a.3 | cagacggcggtccagga | SEQ ID NO:193 |
| Co.a.4 | gacggcggtccaggacaa | SEQ ID NO:194 |
| Co.a.5 | cggcggtccaggacaac | SEQ ID NO:195 |
| Co.a.1.cr | gttgtcctggaccgccgt | SEQ ID NO:196 |
| Co.a.2.cr | tcctggaccgccgtctg | SEQ ID NO:197 |
| Co.a.3.cr | ctggaccgccgtctggt | SEQ ID NO:198 |
| Co.a.4.cr | ggaccgccgtctggttg | SEQ ID NO:199 |
| Co.a.5.cr | accgccgtctggttgtt | SEQ ID NO:200 |
| Colton-b-allele: | | |
| Co.b.1 | ggaacaaccagacggtggt | SEQ ID NO:201 |
| Co.b.2 | acaaccagacggtggtccag | SEQ ID NO:202 |
| Co.b.3 | accagacggtggtccagga | SEQ ID NO:203 |
| Co.b.4 | gacggtggtccaggacaacg | SEQ ID NO:204 |
| Co.b.5 | cggtggtccaggacaacg | SEQ ID NO:205 |
| Co.b.1.cr | cgttgtcctggaccaccgt | SEQ ID NO:206 |
| Co.b.2.cr | ttgtcctggaccaccgtctg | SEQ ID NO:207 |
| Co.b.3.cr | ctggaccaccgtctggttgt | SEQ ID NO:208 |
| Co.b.4.cr | ggaccaccgtctggttgttc | SEQ ID NO:209 |
| Co.b.5.cr | accaccgtctggttgttcc | SEQ ID NO:210 |
| Diego-a-allele: | | |
| Di.a.1 | gtgaagtccacgccggc | SEQ ID NO:211 |
| Di.a.2 | gaagtccacgccggcct | SEQ ID NO:212 |
| Di.a.3 | agtccacgccggcctcc | SEQ ID NO:213 |
| Di.a.4 | tccacgccggcctccct | SEQ ID NO:214 |
| Di.a.5 | acgccggcctccctggcc | SEQ ID NO:215 |
| Di.a.1.cr | gccagggaggccggcgt | SEQ ID NO:216 |
| Di.a.2.cr | agggaggccggcgtgga | SEQ ID NO:217 |
| Di.a.3.cr | ggaggccggcgtggact | SEQ ID NO:218 |
| Di.a.4.cr | aggccggcgtggacttc | SEQ ID NO:219 |
| Di.a.5.cr | ggccggcgtggacttca | SEQ ID NO:220 |
| Diego-b-allele: | | |
| Di.b.1 | ggtgaagtccacgctggc | SEQ ID NO:221 |
| Di.b.2 | gtgaagtccacgctggcct | SEQ ID NO:222 |
| Di.b.3 | tgaagtccacgctggcctcc | SEQ ID NO:223 |
| Di.b.4 | gaagtccacgctggcctccct | SEQ ID NO:224 |
| Di.b.5 | acgctggcctccctggccc | SEQ ID NO:225 |
| Di.b.1.cr | ggccagggaggccagcgt | SEQ ID NO:226 |
| Di.b.2.cr | cagggaggccagcgtgga | SEQ ID NO:227 |
| Di.b.3.cr | agggaggccagcgtggact | SEQ ID NO:228 |
| Di.b.4.cr | ggaggccagcgtggacttc | SEQ ID NO:229 |
| Di.b.5.cr | ggccagcgtggacttcacc | SEQ ID NO:230 |
| Diego Wr-a-allele: | | |
| Wr.a.1 | tgggcttgcgttccaagt | SEQ ID NO:231 |
| Wr.a.2 | ggcttgcgttccaagtttc | SEQ ID NO:232 |
| Wr.a.3 | ttgcgttccaagtttccca | SEQ ID NO:233 |
| Wr.a.4 | cgttccaagtttcccatct | SEQ ID NO:234 |
| Wr.a.5 | tccaagtttcccatctgga | SEQ ID NO:235 |
| Wr.a.1 CR | tccagatgggaaacttgga | SEQ ID NO:236 |
| Wr.a.2 CR | agatgggaaacttggaacg | SEQ ID NO:237 |
| Wr.a.3 CR | tgggaaacttggaacgcaa | SEQ ID NO:238 |
| Wr.a.4 CR | gaaacttggaacgcaagcc | SEQ ID NO:239 |
| Wr.a.5 CR | acttggaacgcaagccca | SEQ ID NO:240 |
| Diego Wr-b-allele:2 | | |
| Wr.b.1 | gggcttgcgttccgagtt | SEQ ID NO:241 |
| Wr.b.2 | gcttgcgttccgagtttc | SEQ ID NO:242 |
| Wr.b.3 | ttgcgttccgagtttccc | SEQ ID NO:243 |
| Wr.b.4 | cgttccgagtttcccatc | SEQ ID NO:244 |
| Wr.b.5 | tccgagtttcccatctgg | SEQ ID NO:245 |
| Wr.b.1 CR | ccagatgggaaactcgga | SEQ ID NO:246 |
| Wr.b.2 CR | gatgggaaactcggaacg | SEQ ID NO:247 |
| Wr.b.3 CR | gggaaactcggaacgcaa | SEQ ID NO:248 |
| Wr.b.4 CR | gaaactcggaacgcaagc | SEQ ID NO:249 |
| Wr.b.5 CR | aactcggaacgcaagccc | SEQ ID NO:250 |
| Dombrock-a-allele: | | |
| Do.a.1 | taccacccaagaggaaact | SEQ ID NO:251 |
| Do.a.2 | ccacccaagaggaaactgg | SEQ ID NO:252 |
| Do.a.3 | acccaagaggaaactggttg | SEQ ID NO:253 |
| Do.a.4 | caagaggaaactggttgca | SEQ ID NO:254 |
| Do.a.5 | aggaaactggttgcagttga | SEQ ID NO:255 |
| Do a 6 cr | ctcaactgcaaccagtttcc | SEQ ID NO:256 |
| Do a 7 cr | caactgcaaccagtttcctc | SEQ ID NO:257 |
| Do a 8 cr | tgcaaccagtttcctcttgg | SEQ ID NO:258 |
| Do a 9 cr | accagtttcctcttgggtgg | SEQ ID NO:259 |
| Do a 10 cr | cagtttcctcttgggtggta | SEQ ID NO:260 |
| Dombrock-b-allele: | | |
| Do.b.1 | taccacccaagaggagact | SEQ ID NO:261 |
| Do.b.2 | ccacccaagaggagactgg | SEQ ID NO:262 |
| Do.b.3 | acccaagaggagactggttg | SEQ ID NO:263 |
| Do.b.4 | caagaggagactggttgca | SEQ ID NO:264 |
| Do.b.5 | aggagactggttgcagttga | SEQ ID NO:265 |
| Do b 6 cr | ctcaactgcaaccagtctcc | SEQ ID NO:266 |
| Do b 7 cr | caactgcaaccagtctcctc | SEQ ID NO:267 |
| Do b 8 cr | tgcaaccagtctcctcttgg | SEQ ID NO:268 |
| Do b 9 cr | accagtctcctcttgggtgg | SEQ ID NO:269 |
| Do b 10 cr | cagtctcctcttgggtggt | SEQ ID NO:270 |
| Dombrock-Joseph(a) positive allele: | | |
| Jo.a.pos.1 | ccccagaacatgactaccac | SEQ ID NO:271 |
| Jo.a.pos.2 | ccagaacatgactaccacaca | SEQ ID NO:272 |
| Jo.a.pos.3 | agaacatgactaccacacacgc | SEQ ID NO:273 |
| Jo.a.pos.4 | catgactaccacacacgctgt | SEQ ID NO:274 |
| Jo.a.pos.5 | actaccacacacgctgtgg | SEQ ID NO:275 |
| Jo.a.pos.1.cr | gccacagcgtgtgtggtagt | SEQ ID NO:276 |
| Jo.a.pos.2.cr | acagcgtgtgtggtagtcatg | SEQ ID NO:277 |
| Jo.a.pos.3.cr | agcgtgtgtggtagtcatgtt | SEQ ID NO:278 |
| Jo.a.pos.4.cr | cgtgtgtggtagtcatgttctg | SEQ ID NO:279 |
| Jo.a.pos.5.cr | gtggtagtcatgttctgggg | SEQ ID NO:280 |
| Dombrock-Joseph(a) negative allele: | | |
| Jo.a.neg.1 | ctaccccagaacatgactatcac | SEQ ID NO:281 |
| Jo.a.neg.2 | cccagaacatgactatcacaca | SEQ ID NO:282 |
| Jo.a.neg.3 | cagaacatgactatcacacacgc | SEQ ID NO:283 |
| Jo.a.neg.4 | catgactatcacacacgctgtg | SEQ ID NO:284 |
| Jo.a.neg.5 | actatcacacacgctgtggc | SEQ ID NO:285 |
| Jo.a.neg.1.cr | aatagccacagcgtgtgtgatagt | SEQ ID NO:286 |
| Jo.a.neg.2.cr | cacagcgtgtgtgatagtcatg | SEQ ID NO:287 |
| Jo.a.neg.3.cr | cagcgtgtgtgatagtcatgtt | SEQ ID NO:288 |
| Jo.a.neg.4.cr | cgtgtgtgatagtcatgttctgg | SEQ ID NO:289 |
| Jo.a.neg.5.cr | gtgatagtcatgttctggggtag | SEQ ID NO:290 |
| Duffy-a-allele: | | |
| Fy.A.1 | ccagatggagactatggtgcc | SEQ ID NO:291 |
| Fy.A.2 | atggagactatggtgccaac | SEQ ID NO:292 |
| Fy.A.3 | ggagactatggtgccaacctg | SEQ ID NO:293 |
| Fy.A.4 | gactatggtgccaacctgga | SEQ ID NO:294 |
| Fy.A.5 | tatggtgccaacctggaag | SEQ ID NO:295 |
| Fy.A.1.cr | cttccaggttggcaccata | SEQ ID NO:296 |
| Fy.A.2.cr | tccaggttggcaccatagtc | SEQ ID NO:297 |
| Fy.A.3.cr | aggttggcaccatagtctcc | SEQ ID NO:298 |
| Fy.A.4.cr | gttggcaccatagtctccat | SEQ ID NO:299 |
| Fy.A.5.cr | gcaccatagtctccatctgg | SEQ ID NO:300 |
| Duffy-b-allele: | | |
| Fy.B.1 | cccagatggagactatgatgcc | SEQ ID NO:301 |
| Fy.B.2 | gatggagactatgatgccaac | SEQ ID NO:302 |
| Fy.B.3 | tggagactatgatgccaacctg | SEQ ID NO:303 |
| Fy.B.4 | gactatgatgccaacctggaa | SEQ ID NO:304 |
| Fy.B.5 | tatgatgccaacctggaagc | SEQ ID NO:305 |
| Fy.B.1.cr | gcttccaggttggcatcata | SEQ ID NO:306 |
| Fy.B.2.cr | ttccaggttggcatcatagtc | SEQ ID NO:307 |
| Fy.B.3.cr | caggttggcatcatagtctcc | SEQ ID NO:308 |
| Fy.B.4.cr | gttggcatcatagtctccatc | SEQ ID NO:309 |
| Fy.B.5.cr | gcatcatagtctccatctggg | SEQ ID NO:310 |
| Duffy GATAbox-normal-allele: | | |
| Fy.GATA.normal.1 | agtccttggctcttatcttg | SEQ ID NO:311 |
| Fy.GATA.normal.2 | agtccttggctcttatcttgga | SEQ ID NO:312 |
| Fy.GATA.normal.3 | cttggctcttatcttggaagc | SEQ ID NO:313 |
| Fy.GATA.normal.4 | gctcttatcttggaagcacagg | SEQ ID NO:314 |
| Fy.GATA.normal.5 | cttatcttggaagcacaggcgc | SEQ ID NO:315 |
| Fy.GATA.normal.1.cr | gcctgtgcttccaagataag | SEQ ID NO:316 |
| Fy.GATA.normal.2.cr | tgtgcttccaagataagagc | SEQ ID NO:317 |
| Fy.GATA.normal.3.cr | tgcttccaagataagagcca | SEQ ID NO:318 |
| Fy.GATA.normal.4.cr | ttccaagataagagccaagga | SEQ ID NO:319 |
| Fy.GATA.normal.5.cr | caagataagagccaaggact | SEQ ID NO:320 |
| Duffy GATAbox-mutation-allele: | | |
| Fy.GATA.mut.1 | tccttggctcttaccttg | SEQ ID NO:321 |
| Fy.GATA.mut.2 | tccttggctcttaccttgga | SEQ ID NO:322 |
| Fy.GATA.mut.3 | tggctcttaccttggaagc | SEQ ID NO:323 |
| Fy.GATA.mut.4 | gctcttaccttggaagcacag | SEQ ID NO:324 |
| Fy.GATA.mut.5 | cttaccttggaagcacaggcg | SEQ ID NO:325 |
| Fy.GATA.mut.1.cr | cctgtgcttccaaggtaag | SEQ ID NO:326 |
| Fy.GATA.mut.2.cr | gtgcttccaaggtaagagc | SEQ ID NO:327 |
| Fy.GATA.mut.3.cr | gcttccaaggtaagagcca | SEQ ID NO:328 |
| Fy.GATA.mut.4.cr | ttccaaggtaagagccaagg | SEQ ID NO:329 |
| Fy.GATA.mut.5.cr | caaggtaagagccaagga | SEQ ID NO:330 |
| Duffy Fyx-normal-allele: | | |
| Fy.X.(b).1 | ttttcagacctctcttccgct | SEQ ID NO:331 |
| Fy.X.(b).2 | agacctctcttccgctggc | SEQ ID NO:332 |
| Fy.X.(b).3 | ctctcttccgctggcagc | SEQ ID NO:333 |
| Fy.X.(b).4 | ctcttccgctggcagctc | SEQ ID NO:334 |
| Fy.X.(b).5 | cttccgctggcagctctg | SEQ ID NO:335 |
| Fy.X.(b).1.cr | cagagctgccagcggaa | SEQ ID NO:336 |
| Fy.X.(b).2.cr | agctgccagcggaagag | SEQ ID NO:337 |
| Fy.X.(b).3.cr | ctgccagcggaagagagg | SEQ ID NO:338 |
| Fy.X.(b).4.cr | gccagcggaagagaggtc | SEQ ID NO:339 |
| Fy.X.(b).5.cr | cagcggaagagaggtctg | SEQ ID NO:340 |
| Duffy Fyx-mutation-allele: | | |
| Fy.X.1 | gcttttcagacctctcttctgct | SEQ ID NO:341 |
| Fy.X.2 | tcagacctctcttctgctggc | SEQ ID NO:342 |
| Fy.X.3 | acctctcttctgctggcagc | SEQ ID NO:343 |
| Fy.X.4 | ctcttctgctggcagctctg | SEQ ID NO:344 |
| Fy.X.5 | cttctgctggcagctctgc | SEQ ID NO:345 |
| Fy.X.1.cr | gcagagctgccagcagaa | SEQ ID NO:346 |
| Fy.X.2.cr | gagctgccagcagaagagag | SEQ ID NO:347 |
| Fy.X.3.cr | agctgccagcagaagagagg | SEQ ID NO:348 |
| Fy.X.4.cr | gccagcagaagagaggtctg | SEQ ID NO:349 |
| Fy.X.5.cr | cagcagaagagaggtctgaaa | SEQ ID NO:350 |
| Kidd-allele-a: | | |
| Jk.a.1 | cagccccatttgaggaca | SEQ ID NO:351 |
| Jk.a.2 | gccccatttgaggacatcta | SEQ ID NO:352 |
| Jk.a.3 | ccatttgaggacatctactttg | SEQ ID NO:353 |
| Jk.a.4 | atttgaggacatctactttgga | SEQ ID NO:354 |
| Jk.a.5 | gaggacatctactttggactct | SEQ ID NO:355 |
| Jk.a.1.cr | cagagtccaaagtagatgtcctc | SEQ ID NO:356 |
| Jk.a.2.cr | agtccaaagtagatgtcctcaaa | SEQ ID NO:357 |
| Jk.a.3.cr | aaagtagatgtcctcaaatggg | SEQ ID NO:358 |
| Jk.a.4.cr | tagatgtcctcaaatggggc | SEQ ID NO:359 |
| Jk.a.5.cr | atgtcctcaaatggggctg | SEQ ID NO:360 |
| Kidd-allele-b: | | |
| Jk.b.1 | tcagccccatttgagaaca | SEQ ID NO:361 |
| Jk.b.2 | gccccatttgagaacatcta | SEQ ID NO:362 |
| Jk.b.3 | cccatttgagaacatctactttg | SEQ ID NO:363 |
| Jk.b.4 | atttgagaacatctactttggac | SEQ ID NO:364 |
| Jk.b.5 | gagaacatctactttggactctg | SEQ ID NO:365 |
| Jk.b.1.cr | ccagagtccaaagtagatgttctc | SEQ ID NO:366 |
| Jk.b.2.cr | agagtccaaagtagatgttctcaaa | SEQ ID NO:367 |
| Jk.b.3.cr | ccaaagtagatgttctcaaatgg | SEQ ID NO:368 |
| Jk.b.4.cr | agtagatgttctcaaatggggc | SEQ ID NO:369 |
| Jk.b.5.cr | atgttctcaaatggggctga | SEQ ID NO:370 |
| Kell-K1-allele: | | |
| KEL.1.1 | tccttaaactttaaccgaatgct | SEQ ID NO:371 |
| KEL.1.2 | ttaaactttaaccgaatgctgaga | SEQ ID NO:372 |
| KEL.1.3 | aactttaaccgaatgctgagactt | SEQ ID NO:373 |
| KEL.1.4 | aaccgaatgctgagacttctg | SEQ ID NO:374 |
| KEL.1.5 | cgaatgctgagacttctgatgag | SEQ ID NO:375 |
| KEL 1.6 CR | actcatcagaagtctcagcattc | SEQ ID NO:376 |
| KEL 1.7 CR | tcagaagtctcagcattcggt | SEQ ID NO:377 |
| KEL 1.8 CR | aagtctcagcattcggttaaag | SEQ ID NO:378 |
| KEL 1.9 CR | tctcagcattcggttaaagtttaa | SEQ ID NO:379 |
| KEL 1.10 CR | agcattcggttaaagtttaagga | SEQ ID NO:380 |
| Kell-K2-allele: | | |
| KEL.2.1 | ccttaaactttaaccgaacgct | SEQ ID NO:381 |
| KEL.2.2 | aactttaaccgaacgctgaga | SEQ ID NO:382 |
| KEL.2.3 | ctttaaccgaacgctgagactt | SEQ ID NO:383 |
| KEL.2.4 | aaccgaacgctgagacttct | SEQ ID NO:384 |
| KEL.2.5 | cgaacgctgagacttctgatg | SEQ ID NO:385 |
| KEL 2.6 CR | tcatcagaagtctcagcgttc | SEQ ID NO:386 |
| KEL 2.7 CR | cagaagtctcagcgttcggt | SEQ ID NO:387 |
| KEL 2.8 CR | agtctcagcgttcggttaaag | SEQ ID NO:388 |
| KEL 2.9 CR | tctcagcgttcggttaaagtt | SEQ ID NO:389 |
| KEL 2.10 CR | agcgttcggttaaagtttaagg | SEQ ID NO:390 |
| Kell-K3-allele: | | |
| KEL.3.1 | aatctccatcacttcatggct | SEQ ID NO:391 |
| KEL.3.2 | tccatcacttcatggctgtt | SEQ ID NO:392 |
| KEL.3.3 | atcacttcatggctgttcca | SEQ ID NO:393 |
| KEL.3.4 | acttcatggctgttccagtt | SEQ ID NO:394 |
| KEL.3.5 | tcatggctgttccagtttc | SEQ ID NO:395 |
| KEL.3.1.cr | agaaactggaacagccatgaa | SEQ ID NO:396 |
| KEL.3.2.cr | aactggaacagccatgaagtg | SEQ ID NO:397 |
| KEL.3.3.cr | tggaacagccatgaagtgatg | SEQ ID NO:398 |
| KEL.3.4.cr | acagccatgaagtgatggag | SEQ ID NO:399 |
| KEL.3.5.cr | gccatgaagtgatggagatt | SEQ ID NO:400 |
| Kell-K4-allele: | | |
| KEL.4.1 | tctccatcacttcacggct | SEQ ID NO:401 |
| KEL.4.2 | ccatcacttcacggctgtt | SEQ ID NO:402 |
| KEL.4.3 | cacttcacggctgttcca | SEQ ID NO:403 |
| KEL.4.4 | acttcacggctgttccag | SEQ ID NO:404 |
| KEL.4.5 | tcacggctgttccagttt | SEQ ID NO:405 |
| KEL.4.1.cr | aaactggaacagccgtgaa | SEQ ID NO:406 |
| KEL.4.2.cr | ctggaacagccgtgaagtg | SEQ ID NO:407 |
| KEL.4.3.cr | ggaacagccgtgaagtgatg | SEQ ID NO:408 |
| KEL.4.4.cr | acagccgtgaagtgatgg | SEQ ID NO:409 |
| KEL.4.5.cr | gccgtgaagtgatggaga | SEQ ID NO:410 |
| Kell-K6-allele: | | |
| KEL.6.1 | tactgcctgggggctgccccgcc | SEQ ID NO:411 |
| KEL.6.2 | tgggggctgccccgcctgt | SEQ ID NO:412 |
| KEL.6.3 | gctgccccgcctgtgac | SEQ ID NO:413 |
| KEL.6.4 | ctgccccgcctgtgacaa | SEQ ID NO:414 |
| KEL.6.5 | gccccgcctgtgacaac | SEQ ID NO:415 |
| KEL.6.1.cr | gttgtcacaggcggggc | SEQ ID NO:416 |
| KEL.6.2.cr | ttgtcacaggcggggcag | SEQ ID NO:417 |
| KEL.6.3.cr | tcacaggcggggcagccc | SEQ ID NO:418 |
| KEL.6.4.cr | acaggcggggcagccccca | SEQ ID NO:419 |
| KEL.6.5.cr | aggcggggcagccccca | SEQ ID NO:420 |
| Kell-K7-allele: | | |
| KEL.7.1 | actgcctgggggctgcctcgcc | SEQ ID NO:421 |
| KEL.7.2 | cctgggggctgcctcgcctgt | SEQ ID NO:422 |
| KEL.7.3 | ggctgcctcgcctgtgac | SEQ ID NO:423 |
| KEL.7.4 | ctgcctcgcctgtgacaacc | SEQ ID NO:424 |
| KEL.7.5 | gcctcgcctgtgacaacc | SEQ ID NO:425 |
| KEL.7.1.cr | ggttgtcacaggcgaggc | SEQ ID NO:426 |
| KEL.7.2.cr | ggttgtcacaggcgaggcag | SEQ ID NO:427 |
| KEL.7.3.cr | ttgtcacaggcgaggcagccc | SEQ ID NO:428 |
| KEL.7.4.cr | acaggcgaggcagcccccagg | SEQ ID NO:429 |
| KEL.7.5.cr | aggcgaggcagcccccagg | SEQ ID NO:430 |
| Lutheran a-allele: | | |
| Lu.a.1 | ggagctcgcccccgcct | SEQ ID NO:431 |
| Lu.a.2 | gctcgcccccgcctagc | SEQ ID NO:432 |
| Lu.a.3 | cgcccccgcctagcctc | SEQ ID NO:433 |
| Lu.a.4 | cccccgcctagcctcgg | SEQ ID NO:434 |
| Lu.a.5 | cccgcctagcctcggct | SEQ ID NO:435 |
| Lu.a.1 CR | agccgaggctaggcggg | SEQ ID NO:436 |
| Lu.a.2 CR | ccgaggctaggcggggg | SEQ ID NO:437 |
| Lu.a.3 CR | gaggctaggcgggggcg | SEQ ID NO:438 |
| Lu.a.4 CR | gctaggcgggggcgagc | SEQ ID NO:439 |
| Lu.a.5 CR | aggcgggggcgagctcc | SEQ ID NO:440 |
| Lutheran b-allele: | | |
| Lu.b.1 | gggagctcgcccccacct | SEQ ID NO:441 |
| Lu.b.2 | gagctcgcccccacctagc | SEQ ID NO:442 |
| Lu.b.3 | ctcgcccccacctagcctc | SEQ ID NO:443 |
| Lu.b.4 | cccccacctagcctcggct | SEQ ID NO:444 |
| Lu.b.5 | cccacctagcctcggctga | SEQ ID NO:445 |
| Lu.b.1 CR | tcagccgaggctaggtggg | SEQ ID NO:446 |
| Lu.b.2 CR | agccgaggctaggtggggg | SEQ ID NO:447 |
| Lu.b.3 CR | gaggctaggtgggggcgag | SEQ ID NO:448 |
| Lu.b.4 CR | gctaggtgggggcgagctc | SEQ ID NO:449 |
| Lu.b.5 CR | aggtgggggcgagctccc | SEQ ID NO:450 |
| MNS M-allele: | | |
| M.1 | gtgagcatatcagcatcaag | SEQ ID NO:451 |
| M.2 | atcagcatcaagtaccactgg | SEQ ID NO:452 |
| M.3 | catcaagtaccactggtgtg | SEQ ID NO:453 |
| M.4 | taccactggtgtggcaatgc | SEQ ID NO:454 |
| M.5 | ctggtgtggcaatgcaca | SEQ ID NO:455 |
| M.1.cr | tgtgcattgccacaccagt | SEQ ID NO:456 |
| M.2.cr | gcattgccacaccagtggta | SEQ ID NO:457 |
| M.3.cr | ccacaccagtggtacttgatg | SEQ ID NO:458 |
| M.4.cr | accagtggtacttgatgct | SEQ ID NO:459 |
| M.5.cr | cttgatgctgatatgctcac | SEQ ID NO:460 |
| MNS N-allele: | | |
| N.1 | tgtgagcatatcagcattaag | SEQ ID NO:461 |
| N.2 | atcagcattaagtaccactgagg | SEQ ID NO:462 |
| N.3 | cattaagtaccactgaggtgg | SEQ ID NO:463 |
| N.4 | accactgaggtggcaatgc | SEQ ID NO:464 |
| N.5 | ctgaggtggcaatgcacact | SEQ ID NO:465 |
| N.1.cr | gtgtgcattgccacctcagt | SEQ ID NO:466 |
| N.2.cr | gcattgccacctcagtggta | SEQ ID NO:467 |
| N.3.cr | ccacctcagtggtacttaatgc | SEQ ID NO:468 |
| N.4.cr | cctcagtggtacttaatgct | SEQ ID NO:469 |
| N.5.cr | cttaatgctgatatgctcaca | SEQ ID NO:470 |
| MNS S-allele: | | |
| big.S.1 | tttgctttataggagaaatggga | SEQ ID NO:471 |
| big.S.2 | ctttataggagaaatgggaca | SEQ ID NO:472 |
| big.S.3 | ttataggagaaatgggacaacttg | SEQ ID NO:473 |
| big.S.4 | gagaaatgggacaacttgtcc | SEQ ID NO:474 |
| big.S.5 | aaatgggacaacttgtccatc | SEQ ID NO:475 |
| big.S.1.cr | gatggacaagttgtcccattt | SEQ ID NO:476 |
| big.S.2.cr | gacaagttgtcccatttctcc | SEQ ID NO:477 |
| big.S.3.cr | aagttgtcccatttctcctata | SEQ ID NO:478 |
| bigS.4.cr | tgtcccatttctcctataaagca | SEQ ID NO:479 |
| big.S.5.cr | cccatttctcctataaagcaaaa | SEQ ID NO:480 |
| MNS s-allele: | | |
| little.s.1 | tgctttataggagaaacggga | SEQ ID NO:481 |
| little.s.2 | tttataggagaaacgggaca | SEQ ID NO:482 |
| little.s.3 | ggagaaacgggacaacttg | SEQ ID NO:483 |
| little.s.4 | gagaaacgggacaacttgtc | SEQ ID NO:484 |
| little.s.5 | aaacgggacaacttgtccat | SEQ ID NO:485 |
| little.s.1.cr | tggacaagttgtcccgttt | SEQ ID NO:486 |
| little.s.2.cr | acaagttgtcccgtttctcc | SEQ ID NO:487 |
| little.s.3.cr | agttgtcccgtttctcctata | SEQ ID NO:488 |
| little.s.4.cr | tgtcccgtttctcctataaagc | SEQ ID NO:489 |
| little.s.5.cr | cccgtttctcctataaagca | SEQ ID NO:490 |
| MNS U-positive-allele: | | |
| U.pos.1 | ttgctgctctctttagctcc | SEQ ID NO:491 |
| U.pos.2 | ctctctttagctcctgtagtgat | SEQ ID NO:492 |
| U.pos.3 | agctcctgtagtgataatactca | SEQ ID NO:493 |
| U.pos.4 | gtagtgataatactcattatttttg | SEQ ID NO:494 |
| U.pos.5 | taatactcattatttttggggtg | SEQ ID NO:495 |
| U.pos.1.cr | caccccaaaaataatgagtatta | SEQ ID NO:496 |
| U.pos.2.cr | caaaaataatgagtattatcactaca | SEQ ID NO:497 |
| U.pos.3.cr | agtattatcactacaggagctaaa | SEQ ID NO:498 |
| U.pos.4.cr | atcactacaggagctaaagag | SEQ ID NO:499 |
| U.pos.5.cr | gagctaaagagagcagcaaa | SEQ ID NO:500 |
| MNS U-negative-allele: | | |
| U.neg.1 | ttttgctgctctctttatctcc | SEQ ID NO:501 |
| U.neg.2 | gctctctttatctcctgtagagat | SEQ ID NO:502 |
| U.neg.3 | tatctcctgtagagataacactca | SEQ ID NO:503 |
| U.neg.4 | gtagagataacactcattattttt | SEQ ID NO:504 |
| U.neg.5 | taacactcattatttttggggt | SEQ ID NO:505 |
| U.neg.1.cr | accccaaaaataatgagtgtta | SEQ ID NO:506 |
| U.neg.2.cr | aaaaataatgagtgttatctctaca | SEQ ID NO:507 |
| U.neg.3.cr | agtgttatctctacaggagataaa | SEQ ID NO:508 |
| U.neg.4.cr | atctctacaggagataaagagag | SEQ ID NO:509 |
| U.neg.5.cr | gagataaagagagcagcaaaatta | SEQ ID NO:510 |
| Rhesus C-allele(307T): | | |
| Rh.big.C.1 | tgagccagttcccttctgg | SEQ ID NO:511 |
| Rh.big.C.2 | gagccagttcccttctgg | SEQ ID NO:512 |
| Rh.big.C.3 | ctgagccagttcccttctg | SEQ ID NO:513 |
| Rh.big.C.4 | ccttctgggaaggtggtc | SEQ ID NO:514 |
| Rh.big.C.5 | ccttctgggaaggtggtca | SEQ ID NO:515 |
| Rh.big.C.1.cr | tgaccaccttcccagaagg | SEQ ID NO:516 |
| Rh.big.C.2.cr | gaccaccttcccagaagg | SEQ ID NO:517 |
| Rh.big.C.3.cr | ccagaagggaactggctc | SEQ ID NO:518 |
| Rh.big.C.4.cr | ccagaagggaactggctca | SEQ ID NO:519 |
| Rh.big.C.5.cr | cagaagggaactggctcag | SEQ ID NO:520 |
| Rhesus c-allele(307C): | | |
| Rh.little.c.1 | gagccagttccctcctgg | SEQ ID NO:521 |
| Rh.little.c.2 | agccagttccctcctgg | SEQ ID NO:522 |
| Rh.little.c.3 | tgagccagttccctcctg | SEQ ID NO:523 |
| Rh.little.c.4 | cctcctgggaaggtggt | SEQ ID NO:524 |
| Rh.little.c.5 | cctcctgggaaggtggtc | SEQ ID NO:525 |
| Rh.little.c.1.cr | gaccaccttcccaggagg | SEQ ID NO:526 |
| Rh.little.c.2.cr | accaccttcccaggagg | SEQ ID NO:527 |
| Rh.little.c.3.cr | ccaggagggaactggct | SEQ ID NO:528 |
| Rh.little.c.4.cr | ccaggagggaactggctc | SEQ ID NO:529 |
| Rh.little.c.5.cr | caggagggaactggctca | SEQ ID NO:530 |
| Rhesus BigC-intron2-specific-insert-allele: | | |
| RhC.intron.2.1 | agggtgccctttgtcacttc | SEQ ID NO:531 |
| RhC.intron.2.2 | gccctttgtcacttcccagt | SEQ ID NO:532 |
| RhC.intron.2.3 | cctttgtcacttcccagtgg | SEQ ID NO:533 |
| RhC.intron.2.4 | ttgtcacttcccagtggtacaa | SEQ ID NO:534 |
| RhC.intron.2.5 | tcacttcccagtggtacaatca | SEQ ID NO:535 |
| RhC.intron.2.1.cr | gaagtgacaaagggcaccct | SEQ ID NO:536 |
| RhC.intron.2.2.cr | actgggaagtgacaaagggc | SEQ ID NO:537 |
| RhC.intron.2.3.cr | ccactgggaagtgacaaagg | SEQ ID NO:538 |
| RhC.intron.2.4.cr | tgtaccactgggaagtgacaaa | SEQ ID NO:539 |
| RhC.intron.2.5.cr | tgattgtaccactgggaagtga | SEQ ID NO:540 |

Rhesus BigC-intron2-specific-insert-negative-allele:

The setup of the PCR is chosen in such a way that a PCR product will be formed only, when the bigC-intron2-specific-insert is present. The antitags (used for background subtraction) are used to calculate the ratio for genotyping.

| Rhesus E-allele: | | |
|---|---|---|
| Rh.big.E.1 | gccaagtgtcaactctcctct | SEQ ID NO:541 |
| Rh.big.E.2 | caagtgtcaactctcctctgct | SEQ ID NO:542 |
| Rh.big.E.3 | gtgtcaactctcctctgctgag | SEQ ID NO:543 |
| Rh.big.E.4 | caactctcctctgctgagaagtc | SEQ ID NO:544 |
| Rh.big.E.5 | tctcctctgctgagaagtcc | SEQ ID NO:545 |
| Rh.big.E.1.cr | ggacttctcagcagaggagag | SEQ ID NO:546 |
| Rh.big.E.2.cr | cttctcagcagaggagagttga | SEQ ID NO:547 |
| Rh.big.E.3.cr | ctcagcagaggagagttgacac | SEQ ID NO:548 |
| Rh.big.E.4.cr | gcagaggagagttgacacttg | SEQ ID NO:549 |
| Rh.big.E.5.cr | gaggagagttgacacttggc | SEQ ID NO:550 |
| Rhesus e-allele: | | |
| Rh.little.e.1 | gccaagtgtcaactctgctct | SEQ ID NO:551 |
| Rh.little.e.2 | caagtgtcaactctgctctgct | SEQ ID NO:552 |
| Rh.little.e.3 | tgtcaactctgctctgctgag | SEQ ID NO:553 |
| Rh.little.e.4 | caactctgctctgctgagaag | SEQ ID NO:554 |
| Rh.little.e.5 | tctgctctgctgagaagtcc | SEQ ID NO:555 |
| Rh.little.e.1.cr | ggacttctcagcagagcagag | SEQ ID NO:556 |
| Rh.little.e.2.cr | ttctcagcagagcagagttga | SEQ ID NO:557 |
| Rh.little.e.3.cr | tcagcagagcagagttgacac | SEQ ID NO:558 |
| Rh.little.e.4.cr | gcagagcagagttgacacttg | SEQ ID NO:559 |
| Rh.little.e.5.cr | gagcagagttgacacttggc | SEQ ID NO:560 |
| Rhesus RHD-allele: | | |
| RhD 1 | ttccccacagctccatcat | SEQ ID NO:561 |
| RhD 2 | acagctccatcatgggctac | SEQ ID NO:562 |
| RhD 3 | tccatcatgggctacaacttc | SEQ ID NO:563 |
| RbD 4 | tcatgggctacaacttcagct | SEQ ID NO:564 |
| RhD 5 | gggctacaacttcagcttgct | SEQ ID NO:565 |
| RhD cr 1 | agcaagctgaagttgtagccc | SEQ ID NO:566 |
| RbD cr 2 | agctgaagttgtagcccatga | SEQ ID NO:567 |
| RbD cr 3 | gaagttgtagcccatgatgg | SEQ ID NO:568 |
| RhD cr 4 | gcccatgatggagctgt | SEQ ID NO:569 |
| RhD cr 5 | tgatggagctgtggggaa | SEQ ID NO:570 |

Rhesus RHD-negative-allele:

The setup of the PCR is chosen in such a way that a PCR product will be formed only, when the RHD gene is present. The antitags (used for background subtraction) are used to calculate the ratio for genotyping.

Rhesus r's-allele:

| | | |
|---|---|---|
| r′s.T.1 | ggaaggtcaacttggtgca | SEQ ID NO:571 |
| r′s.T.2 | ggaaggtcaacttggtgcagt | SEQ ID NO:572 |
| r′s.T.3 | caacttggtgcagttggtg | SEQ ID NO:573 |
| r′s.T.4 | acttggtgcagttggtggt | SEQ ID NO:574 |
| r′s.T.5 | ttggtgcagttggtggtgat | SEQ ID NO:575 |
| r′s.T.1.cr | catcaccaccaactgcacca | SEQ ID NO:576 |
| r′s.T.2.cr | caccaccaactgcaccaagt | SEQ ID NO:577 |
| r′s.T.3.cr | accaactgcaccaagttgac | SEQ ID NO:578 |
| r′s.T.4.cr | actgcaccaagttgaccttcc | SEQ ID NO:579 |
| r′s.T.5.cr | tgcaccaagttgaccttcc | SEQ ID NO:580 |

Rhesus r's-negative-allele:

The setup of the PCR is chosen in such a way that a PCR product will be formed only, when the r's gene is present. The antitags (used for background subtraction) are used to calculate the ratio for genotyping.
Rhesus DVI-Allele:

The setup of the PCR is chosen in such a way that a PCR product will be formed only, when a normal RHD gene is present. The antitags (used for background subtraction) are used to calculate the ratio for genotyping.

| DVI-probes for presence normal RHD allele: | | |
|---|---|---|
| Rh.DVI.1 | atttcaaccctcttggcctt | SEQ ID NO:581 |
| Rh.DVI.2 | aaccctcttggcctttgttt | SEQ ID NO:582 |
| Rh.DVI.3 | tcttggcctttgtttccttg | SEQ ID NO:583 |
| Rh.DVI.4 | ggtatcagcttgagagctcg | SEQ ID NO:584 |
| Rh.DVI.5 | atcagcttgagagctcggag | SEQ ID NO:585 |
| Rh.DVI.1.cr | aaggccaagagggttgaaat | SEQ ID NO:586 |
| Rh.DVI.2.cr | aaacaaaggccaagagggtt | SEQ ID NO:587 |
| Rh.DVI.3.cr | caaggaaacaaaggccaaga | SEQ ID NO:588 |
| Rh.DVI.4.cr | cgagctctcaagctgatacc | SEQ ID NO:589 |
| Rh.DVI.5.cr | ctccgagctctcaagctgat | SEQ ID NO:590 |
| Rhesus RHD-Pseudogene-mutation-allele: | | |
| RhD Y 1 | tttctttgcagacttaggtgc | SEQ ID NO:591 |
| RhD Y 2 | ctttgcagacttaggtgcaca | SEQ ID NO:592 |
| RhD Y 3 | tttgcagacttaggtgcacagt | SEQ ID NO:593 |
| RhD Y 4 | acttaggtgcacagtgcgg | SEQ ID NO:594 |
| RhD Y 5 | cttaggtgcacagtgcggt | SEQ ID NO:595 |
| RhD Y cr 1 | caccgcactgtgcacctaa | SEQ ID NO:596 |
| RhD Y cr 2 | ccgcactgtgcacctaagtc | SEQ ID NO:597 |
| RhD Y cr 3 | gcactgtgcacctaagtctgc | SEQ ID NO:598 |
| RhD Y cr 4 | tgcacctaagtctgcaaaga | SEQ ID NO:599 |
| RhD Y cr 5 | cacctaagtctgcaaagaaatagcg | SEQ ID NO:600 |
| Rhesus RHD-Pseudogene-normal-allele: | | |
| RhD non Y 1 | ctatttctttgcagacttatgtgc | SEQ ID NO:601 |
| RhD non Y 2 | tctttgcagacttatgtgcaca | SEQ ID NO:602 |
| RhD non Y 3 | ttgcagacttatgtgcacagtg | SEQ ID NO:603 |
| RhD non Y 4 | acttatgtgcacagtgcggt | SEQ ID NO:604 |
| RhD non Y 5 | cttatgtgcacagtgcggtg | SEQ ID NO:605 |
| RhD non Y cr 1 | acaccgcactgtgcacataa | SEQ ID NO:606 |
| RhD non Y cr 2 | accgcactgtgcacataagtc | SEQ ID NO:607 |
| RhD non Y cr 3 | gcactgtgcacataagtctgc | SEQ ID NO:608 |
| RhD non Y cr 4 | tgtgcacataagtctgcaaag | SEQ ID NO:609 |
| RhD non Y cr 5 | cacataagtctgcaaagaaatagcg | SEQ ID NO:610 |
| Yt-a-allele: | | |
| Yt.a.1 | gcgggagacttccacgg | SEQ ID NO:611 |
| Yt.a.2 | gggagacttccacggcct | SEQ ID NO:612 |
| Yt.a.3 | gagacttccacggcctgc | SEQ ID NO:613 |
| Yt.a.4 | gacttccacggcctgca | SEQ ID NO:614 |
| Yt.a.5 | ttccacggcctgcaggta | SEQ ID NO:615 |
| Yt.a.1 CR | tacctgcaggccgtggaa | SEQ ID NO:616 |
| Yt.a.2 CR | tgcaggccgtggaagtc | SEQ ID NO:617 |
| Yt.a.3 CR | gcaggccgtggaagtctc | SEQ ID NO:618 |
| Yt.a.4 CR | aggccgtggaagtctccc | SEQ ID NO:619 |
| Yt.a.5 CR | ccgtggaagtctcccgc | SEQ ID NO:620 |
| Yt-b-allele: | | |
| Yt.b.1 | gcgggagacttcaacggc | SEQ ID NO:621 |
| Yt.b.2 | gggagacttcaacggcctg | SEQ ID NO:622 |
| Yt.b.3 | gagacttcaacggcctgca | SEQ ID NO:623 |
| Yt.b.4 | gacttcaacggcctgcag | SEQ ID NO:624 |
| Yt.b.5 | ttcaacggcctgcaggtaa | SEQ ID NO:625 |
| Yt.b.1 CR | ttacctgcaggccgttgaa | SEQ ID NO:626 |
| Yt.b.2 CR | ctgcaggccgttgaagtc | SEQ ID NO:627 |
| Yt.b.3 CR | tgcaggccgttgaagtctc | SEQ ID NO:628 |
| Yt.b.4 CR | caggccgttgaagtctccc | SEQ ID NO:629 |
| Yt.b.5 CR | gccgttgaagtctcccgc | SEQ ID NO:630 |

Using the available software programs, it would be possible for a skilled person to design probes for other blood cell antigen alleles.

The DNA array may be a limited array comprising for example only 72 different probes, but preferably it comprises substantially all probes shown in the above list.

In order to facilitate binding of the probes to the array support and to allow sufficient access to the amplification products, the probe oligos will normally be provided with a linker molecule and a reactive group, preferably at the 5′-end of the oligo, for binding to the array support. All of this is well known to the skilled person and only by way of example it is mentioned that a suitable linker is C6 (hexamethylene, i.e. —(CH₂)₆—) and a suitable reactive group is amino.

Array Support

The probes are located on a suitable support to which they are usually attached by chemical means. The support may be a glass slide or any other suitable support, such as plastic, silicon or porous metal oxide. Usually, the support will be pretreated to improve binding of the oligos, for example pretreated by coating with poly-L-lysine, aminosilane, aldehyde or epoxy. To achieve binding of the probes, various techniques may be used, such as UV irradiation, nonspecific electrostatic absorption, covalent attachment via 5′amino or phosphate group using carbodiimide (EDC) chemistry, etc.

To avoid non-specific hybridization it will usually be preferable to incubate the array support carrying the probes thereon with prehybridization solution, as is well known to a skilled person, and to prepare the probes for hybridization it will be preferred to denature them, for example by heating at about 80° C. or higher.

Controls

In order to allow correction for background it is preferred to include some probes with a sequence not occurring in the DNA of the donor and which may be expected not to bind by hybridization to amplification products. The signal measured for allele-specific probe spots may be corrected by subtracting the signal measured for these background control spots. Suitable examples of such background correction probes or antitags are the following probes a3, a9, a17, a23, a27, a33, a35, a38, a42 and a43:

| | | |
|---|---|---|
| a3 | cagaccataagcacaggcgt, | SEQ ID NO:631 |
| a9 | gctcgtccacagtgcgttat, | SEQ ID NO:632 |
| a17 | cggcgttcaagcaaaccgaa, | SEQ ID NO:633 |
| a23 | gacatatagctccactcaga, | SEQ ID NO:634 |
| a27 | tagggtactgatgagcactc, | SEQ ID NO:635 |
| a33 | tcagccctatcgcaggatgt, | SEQ ID NO:169 |
| a35 | gagacacttgacagtagcca, | SEQ ID NO:170 |
| a38 | ggcagggcacctcagtttat, | SEQ ID NO:171 |
| a42 | tcaccagccagactgtgtag, | SEQ ID NO:172 |
| a43 | cttcacgcaagttgtccaga. | SEQ ID NO:173 |

Further, it may be advisable to include at least one positive control probe. This probe should be the complement of a labeled positive control oligomer added to the products of the amplification. For example, the positive control probe may be the following oligo CS05:

| | | |
|---|---|---|
| CS05 | gtcctgacttctagctcatg, | SEQ ID NO:174 |

which is capable of hybridizing to its labelled complement catgagctagaagtcaggac which is added to the mixture of amplification products before the mixture is applied onto the DNA array.
Hybridization Conditions

Hybridization of products of the amplification to the probes in the DNA array is usually carried out at a temperature of between 50 and 65° C., preferably at about 56° C., for a sufficient time, such as for 15 minutes to 10 hours, more preferably for 30 minutes to 4 hours.

Array Configuration

The configuration of the array is fully arbitrary. Usually, one array support will carry a large number of spots present in several blocks that may be identical (preferred) or different from each other. Each spot contains one probe and it is preferred that similar probes are spotted as distant from each other as possible. It is preferable that each block contains one spot of each of the allele-specific probes, and in addition some control spots. Thus, one block may comprise 128 allele-specific spots, 5 background controls and a positive control, in total 134 spots.

Measuring the Fluorescence Signal

The binding of labelled amplification products to certain spots is determined on the basis of the fluorescence signal emitted by these spots. The signal can be measured with a photomultiplier tube, scanning laser systems or any other apparatus and technique known to the skilled person.

Evaluation of the Resulting Data

To evaluate the resulting data as to the blood cell antigen genotype of the donor of the DNA, the fluorescence signal intensities may be converted using Genepix Pro 5.0 (Axon Instruments Inc.) to signal values which can be analyzed further, for example in Excel.

### EXAMPLES

In the Examples below, that only serve to illustrate the invention without limiting it in any way, the concept of genotyping blood cell antigens by the present combination of a particular multiplex PCR and a DNA microarray of allele-specific probes is tested in Example 1 on the biallelic Human Platelet Antigen systems HPA-1 through 5 and Gov. A blind panel of 58 donor samples was genotyped for these 6 HPA systems and only one discrepancy was found in one HPA system. The discrepancy can be overcome by adjusting the scoring criteria and validating the new format. Then, in Example 2, it was shown that the multiplex PCR of this invention worked well with all primers of Table II together.

### Example 1

Sequences and Tm values of the probes spotted on poly-Lysine-coated glass slide are shown in Table I. The SNPs of HPA-3 and -5 are located in a GC-rich and -poor area, respectively. Therefore, these probes are shorter or longer than the other oligonucleotides, and have a Tm outside the 60-65° C. range. The probes, dissolved at a concentration of 50 μM in 0.4 M NaHCO₃ (pH 9.4), were spotted on poly-L-lysine coated glass slides by the Omnigrid 100® microarrayer (Genemachines) supplied with 16 SMP 3 Microspotting pins (Telechem). Each glass slide contains 48 blocks of 134 spots corresponding to the 128 allele-specific probes, the 5 background controls and the positive control CS05. Similar probes were spotted as distant as possible from each other. DNA was cross-linked by UV irradiation at 250 mJ/cm² (Stratalinker model 1800 UV Illuminator, Stratagene). To prevent non-specific hybridisation, the slides were incubated with 100 μl of prehybridisation solution [400 ng/μl yeast tRNA (Roche), 400 ng/μl herring sperm DNA (Gibco BRL), 5× Denhardt's solution, 3.2×SSC and 0.4% SDS] at 65° C. for 30 min. Prior to hybridisation, the slide containing the prehybridisation mixture was incubated for 2 min at 80° C. to denature the spotted DNA. After prehybridisation, the slides were washed twice in 2×SSC for 5 min at room temperature and dehydrated with a series of 70 (twice), 90 and 100% ethanol, for 5 min each, respectively.

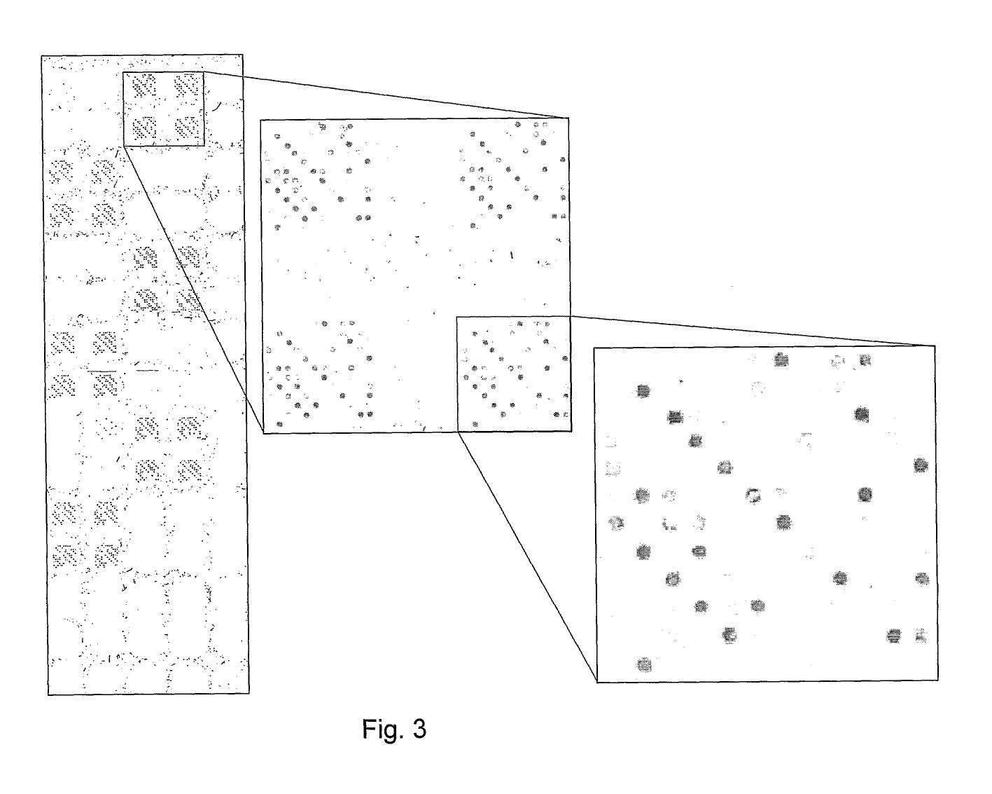

A multiplex PCR for only the six platelet antigens was performed using 5 nM of each HPA and gov primer (7.5 nM of each HPA-3 primer) and 1.2 μM of each Cy5 labeled universal primer. Further, the multiplex PCR was performed as described for the multiplex PCR in Example 2. For hybridisation reaction chambers were made as described by Pastinen et al. (2000). A silicon rubber grid was used to divide the glass slide in 24 different compartments. While the slide was preheated (65° C. for 75 min), 40 μl of the multiplex PCR products was denatured at 95° C. for 5 min, put on ice and 120 μl of ice-cold hybridisation solution [2 ng Cy5-labelled complement of CS05 (5′-catgagctagaagtcaggac-3′) and 4×SSC] was added. For hybridisation, 80 μl of this mixture was added per array and incubated for 3½ hours at 56° C. Next, slides were lifted out of the hybridisation holder in 600 ml 3×SSC and washed twice with 2×SSC/0.1% SDS at 50° C. and twice with 0.2×SSC at RT, for 5 min each, respectively. Glass slides were dried by centrifugation at 800 rpm for 5 min and scanned in an Agilent G2565BA micro array scanner at 10 μm resolution. Photomultiplier tube voltage was always set at 100%. PCR products of six different donors were analysed per slide as shown in FIG. 3. After Genepix analysis the obtained data was converted to Excel. A panel of 12 donor samples with known HPA typing was used to define the HPA scoring criteria. Table III lists the scoring criteria. The median signal intensities F_{med} of the negative controls (C) were averaged and this background value plus three times its standard deviation was subtracted from the median intensity F_{med} of each oligo, giving a threshold value, Fₜₕ, shown in the third column in Table III. If the Fₜₕ showed a negative value for both the matched (allele a) and the mismatched (allele b) probe, the primer pair was excluded for the genotyping result. For genotyping, the ratio of the intensities of two oligonucleotides (one from allele a and the other from allele b) in one set was calculated. The ratios were calculated of the F_{med}−F_{bg} intensities for the a-allele to the sum of the F_{med}−F_{bg} intensities for both alleles in the sixth column. This formula a/(a+b) has also been described by Hinds et al. (2004). In principle, these ratios should take on values near 1.0, 0.5 or 0.0 for aa, ab or bb genotypes. Only positive F_{med}−F_{bg} values were used and therefore negative values were set to 1 in the fifth column. Next, the ratios were converted to genotypes in the seventh column. For HPA-1, -2, -4 and -5 ratio values above 0.75 were typed aa, below 0.25 bb and between 0.35 and 0.65 ab. Due to a high binding of the PCR fragments to the probes representing the a allele, the criteria for HPA-3 and Gov were adjusted to 0.95 and 0.8 for aa, 0.45 and 0.35 for bb and for ab between 0.55 and 0.85 and between 0.45 and 0.7, respectively. The most frequent genotype was coloured orange in the seventh column. This was done for every blood group, every block and every sample. The samples of the 12 donors were used to accept probe-sets that correctly predicted the genotype. As criteria used for the final scoring: 50% of the used probe-sets should predict a genotype if a homozygote genotype was predicted and 40% if a heterozygote genotype was predicted (30% for HPA-3). Using these criteria a blind panel of 58 samples were genotyped. The results of nine of them are shown in Table IV. The genotypes of all 58 samples are listed in Table V. The genotypes of 56 samples could be scored with the preset criteria. Only in one sample a result discrepant with its known genotype was obtained. In this case, 33.3% of the probe sets indicated an HPA-3bb genotype and 37.5% indicated an HPA-3ab genotype as shown in Table VI. Adjustment of the scoring criteria leads to complete concordance of the typing results.

### Example 2

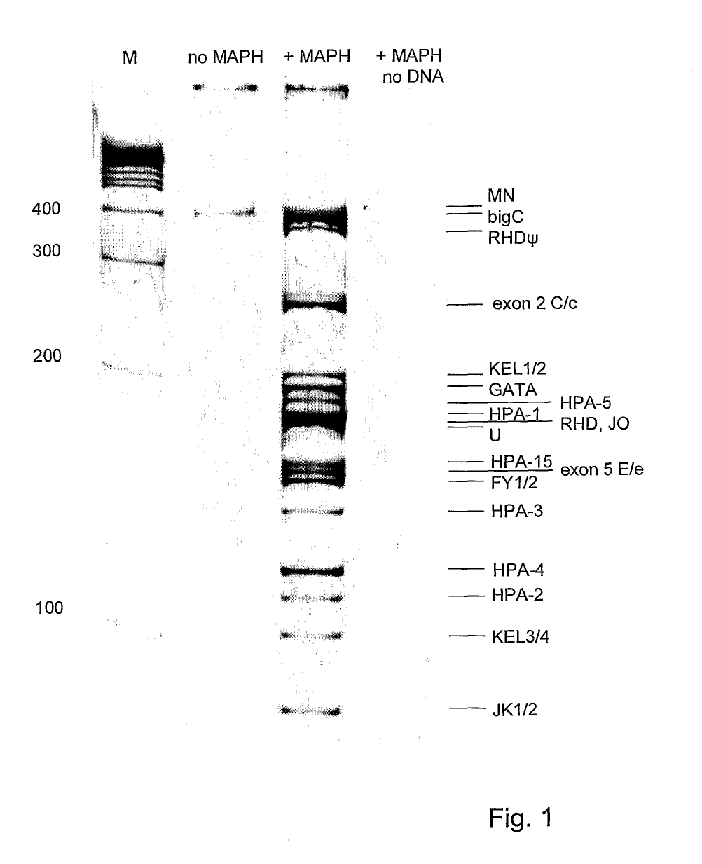

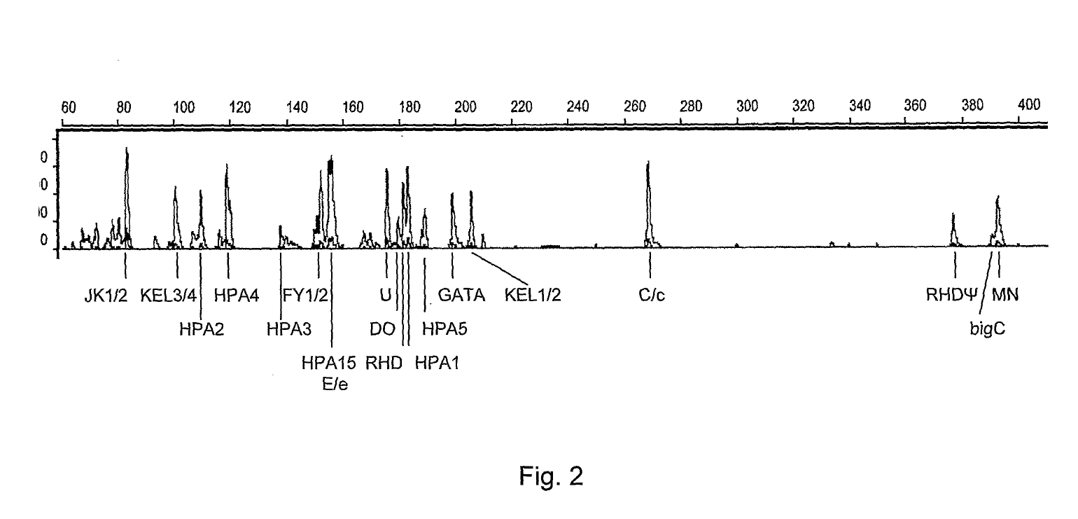

A primer mix was constructed with the primers listed in Table II. The concentration of the primers in the PCR mixture is also listed in Table II. The Qiagen multiplex kit was used for the multiplex PCR. In the multiplex PCR, the annealing temperature was not changed during PCR and two fluorescent labelled universal primers were used. In more detail, at the start the PCR mixture contains a very low amount of chimeric primers (5 nM) and an excess of fluorescent-labelled universal primers (0.2 μM of each universal primer per chimeric primer). The temperature profile of the PCR started with 15 min at 95° C., followed by 45 cycles of 94° C. for 30 sec, 57° C. for 90 sec, 72° C. for 90 sec and the protocol ended with a final polymerization step at 72° C. for 10 min. Very little amount of PCR product is amplified during the first amplification cycles, but enough to be used as template by the universal MAPH primers in the following cycles as can be seen on 8% acrylamide gel in FIG. 1. By tagging the MAPH primers at their 5′end with the fluorescent label Cy5, only two fluorescent-labelled primers are necessary to label the PCR product efficiently. As described by White et al. (2002), a better discrmination of the multiplex PCR products can be visualized on an ABI 3700 capillary sequencer (Applied Biossystems), in this case only MAPH-rev is fluorescent (FAM) labeled. Multiplex PCR with all designed PCR primers yielded the expected pattern of peaks as shown in the chromatogram in FIG. 2. This means that all gene-fragments were amplified at similar yields.

TABLE I

Sequences and melting temperatures of the
probes.

| | | |
|---|---|---|
| Probe | Tm | Sequence (5′-3′) |

| | | | |
|---|---|---|---|
| HPA-1aa | 65.86 | tacaggccctgcctctggg | +TL,32 |
| HPA-1ab | 66.92 | aggccctgcctctgggct | |
| HPA-1ac | 67.79 | ccctgcctctgggctcacc | |
| HPA-1ad | 67.35 | tgcctctgggctcacctcg | |
| HPA-1ae | 64.65 | ctctgggctcacctcgctg | |
| HPA-1aa CR | 64.65 | cagcgaggtgagcccagag | |
| HPA-1ab CR | 67.35 | cgaggtgagcccagaggca | |
| HPA-1ac CR | 67.79 | ggtgagcccagaggcaggg | |
| HPA-1ad CR | 66.92 | agcccagaggcagggcct | |
| HPA-1be CR | 65.86 | cccagaggcagggcctgta | |
| HPA-1ba | 69.93 | tacaggccctgcctccggg | |
| HPA-1bb | 70.94 | aggccctgcctccgggct | |
| HPA-1bc | 68.84 | ccctgcctccgggctcac | |
| HPA-1bd | 66.21 | ctgcctccgggctcacct | |
| HPA-1be | 66.33 | ctccgggctcacctcgct | |
| HPA-1ba CR | 66.33 | agcgaggtgagcccggag | |
| HPA-1bb CR | 66.21 | aggtgagcccggaggcag | |
| HPA-1bc CR | 68.84 | gtgagcccggaggcaggg | |
| HPA-1bd CR | 70.94 | agcccggaggcagggcct | |
| HPA-1be CR | 69.93 | cccggaggcagggcctgta | |
| HPA-2aa | 63.81 | ctgacgcccacacccaag | |
| HPA-2ab | 65.21 | ctcctgacgcccacaccc | |
| HPA-2ac | 65.37 | ggctcctgacgcccacac | |
| HPA-2ad | 66.12 | agggctcctgacgcccac | |
| HPA-2ae | 68.84 | ccagggctcctgacgccc | |
| HPA-2aa CR | 63.81 | cttgggtgtgggcgtcag | |
| HPA-2ab CR | 65.21 | gggtgtgggcgtcaggag | |
| HPA-2ac CR | 65.37 | gtgtgggcgtcaggagcc | |
| HPA-2ad CR | 66.12 | gtgggcgtcaggagccct | |
| HPA-2ae CR | 68.84 | gggcgtcaggagccctgg | |
| HPA-2ba | 64.88 | cctgatgcccacacccaag | |
| HPA-2bb | 65.57 | ctcctgatgcccacaccca | |
| HPA-2bc | 66.37 | ggctcctgatgcccacacc | |
| HPA-2bd | 66.45 | agggctcctgatgcccaca | |
| HPA-2be | 66.45 | ccagggctcctgatgccc | |
| HPA-2ba CR | 64.88 | cttgggtgtgggcatcagg | |
| HPA-2bb CR | 65.57 | tgggtgtgggcatcaggag | |
| HPA-2bc CR | 66.37 | ggtgtgggcatcaggagcc | |
| HPA-2bd CR | 66.45 | tgtgggcatcaggagccct | |
| HPA-2be CR | 66.45 | gggcatcaggagccctgg | |
| HPA-3aa | 69.45 | ccatccccagcccctccc | |
| HPA-3ab | 69.60 | gcccatccccagcccctc | |
| HPA-3ac | 70.57 | ctgcccatccccagcccc | |
| HPA-3ad1 | 70.72 | gctgcccatccccagccc | |
| HPA-3ad | 70.72 | ggctgcccatccccagcc | |
| HPA-3ad2 | 70.72 | gggctgcccatccccagc | |
| HPA-3ae | 70.08 | ggggctgcccatcccca | |
| HPA-3aa CR | 69.45 | gggaggggctggggatgg | |
| HPA-3ab CR | 69.60 | gaggggctggggatgggc | |
| HPA-3ac CR | 70.57 | ggggctggggatgggcag | |
| HPA-3ad1 CR | 70.72 | gggctggggatgggcagc | |
| HPA-3ad CR | 70.72 | ggctggggatgggcagcc | |
| HPA-3ad2 CR | 70.72 | gctggggatgggcagccc | |
| HPA-3ae CR | 70.08 | tggggatgggcagcccc | |
| HPA-3ba | 68.75 | ccagccccagcccctcc | |
| HPA-3bb | 70.39 | gcccagccccagcccct | |
| HPA-3bc | 69.91 | ctgcccagccccagccc | |
| HPA-3bd1 | 70.07 | gctgcccagccccagcc | |
| HPA-3bd | 70.07 | ggctgcccagccccagc | |
| HPA-3bd2 | 69.91 | gggctgcccagccccag | |
| HPA-3be | 72.50 | ggggctgcccagcccca | |
| HPA-3ba CR | 68.75 | ggaggggctggggctgg | |
| HPA-3bb CR | 70.39 | aggggctggggctgggc | |
| HPA-3bc CR | 69.91 | gggctggggctgggcag | |
| HPA-3bd1 CR | 70.07 | ggctggggctgggcagc | |
| HPA-3bd CR | 70.07 | gctggggctgggcagcc | |
| HPA-3bd2 CR | 69.91 | ctggggctgggcagccc | |
| HPA-3be CR | 72.50 | tggggctgggcagcccc | |
| HPA-4aa | 62.77 | gccacccagatgcgaaag | |
| HPA-4ab | 59.94 | cacccagatgcgaaagct | |
| HPA-4ac | 61.08 | cccagatgcgaaagctca | |
| HPA-4ad | 57.99 | cagatgcgaaagctcacc | |
| HPA-4ae | 57.99 | gatgcgaaagctcaccag | |
| HPA-4aa CR | 57.99 | ctggtgagctttcgcatc | |
| HPA-4ab CR | 57.99 | ggtgagctttcgcatctg | |
| HPA-4ac CR | 61.08 | tgagctttcgcatctggg | |
| HPA-4ad CR | 59.94 | agctttcgcatctgggtg | |
| HPA-4ae CR | 62.77 | ctttcgcatctgggtggc | |
| HPA-4ba | 60.20 | gccacccagatgcaaaag | |
| HPA-4bb | 61.19 | ccacccagatgcaaaagct | |
| HPA-4bc | 60.26 | acccagatgcaaaagctcac | |
| HPA-4bd | 58.50 | cagatgcaaaagctcacca | |
| HPA-4be | 58.04 | gatgcaaaagctcaccagtaa | |
| HPA-4ba CR | 58.04 | ttactggtgagcttttgcatc | |
| HPA-4bb CR | 58.50 | tggtgagcttttgcatctg | |
| HPA-4bc CR | 60.26 | gtgagcttttgcatctgggt | |
| HPA-4bd CR | 61.19 | agcttttgcatctgggtgg | |
| HPA-4be CR | 60.20 | cttttgcatctgggtggc | |
| HPA-5aa | 57.46 | gagtctacctgtttactatcaaagagg | |
| HPA-5ab | 56.77 | agtctacctgtttactatcaaagaggta | |
| HPA-5ac | 57.08 | gtctacctgtttactatcaaagaggtaa | |
| HPA-5ad | 57.16 | ctacctgtttactatcaaagaggtaaaa | |
| HPA-5ae | 57.38 | acctgtttactatcaaagaggtaaaaa | |
| HPA-5aa CR | 57.38 | tttttacctctttgatagtaaacaggt | |
| HPA-5ab CR | 57.16 | ttttacctctttgatagtaaacaggtag | |
| HPA-5ac CR | 57.08 | ttacctctttgatagtaaacaggtagac | |
| HPA-5ad CR | 56.77 | tacctctttgatagtaaacaggtagact | |
| HPA-5ae CR | 57.46 | cctctttgatagtaaacaggtagactc | |
| HPA-5ba | 57.42 | gagtctacctgtttactatcaaaaagg | |
| HPA-5bb | 56.75 | agtctacctgtttactatcaaaaaggta | |
| HPA-5bc | 57.05 | gtctacctgtttactatcaaaaaggtaa | |
| HPA-5ad | 57.13 | ctacctgtttactatcaaaaaggtaaaa | |
| HPA-5be | 57.34 | acctgtttactatcaaaaaggtaaaaa | |
| HPA-5ba CR | 57.34 | tttttacctttttgatagtaaacaggt | |
| HPA-5bb CR | 57.13 | ttttacctttttgatagtaaacaggtag | |
| HPA-5bc CR | 57.05 | ttacctttttgatagtaaacaggtagac | |
| HPA-5bd CR | 56.75 | tacctttttgatagtaaacaggtagact | |
| HPA-5be CR | 57.42 | cctttttgatagtaaacaggtagactc | |
| Gov-aa | 58.44 | ttattatcttgacttcagttacaggattt | |
| Gov-ab | 59.20 | tcttgacttcagttacaggatttacc | |
| Gov-ac | 59.15 | tgacttcagttacaggatttaccaa | |
| Gov-ad | 58.36 | cttcagttacaggatttaccaagaat | |
| Gov-ae | 59.44 | cagttacaggatttaccaagaatttg | |
| Gov-aa CR | 59.44 | caaattcttggtaaatcctgtaactg | |
| Gov-ab CR | 58.36 | attcttggtaaatcctgtaactgaag | |
| Gov-ac CR | 59.15 | tggtaaatcctgtaactgaagtcaa | |
| Gov-ad CR | 59.20 | ggtaaatcctgtaactgaagtcaaga | |
| Gov-ae CR | 58.44 | aaatcctgtaactgaagtcaagataataa | |
| Gov-ba | 58.35 | tatcttgacttcagttccaggatt | |
| Gov-bb | 58.39 | cttgacttcagttccaggatttac | |
| Gov-bc | 59.51 | gacttcagttccaggatttacca | |
| Gov-bd | 58.24 | ttcagttccaggatttaccaag | |
| Gov-be | 59.79 | cagttccaggatttaccaagaatt | |
| Gov-ba CR | 59.79 | aattcttggtaaatcctggaactg | |
| Gov-bb CR | 58.24 | cttggtaaatcctggaactgaa | |
| Gov-bc CR | 59.51 | ggtaaatcctggaactgaac | |
| Gov-bd CR | 58.39 | gtaaatcctggaactgaagtcaag | |
| Gov-be CR | 58.35 | aatcctggaactgaagtcaagata | |
| a33 | 63.03 | tcagccctatcgcaggatgt | |
| a35 | 53.75 | gagacacttgacagtagcca | |
| a38 | 61.40 | ggcagggcacctcagtttat | |
| a42 | 57.37 | tcaccagccagactgtgtag | |
| a43 | 60.02 | cttcacgcaagttgtccaga | |
| CS05 | 52.41 | gtcctgacttctagctcatg | |

Each oligonucleotide probe has an amine group and a C6 spacer at its
The polymorphic sites in the allele-specific probes are highlighted.

TABLE II

Primers used for multiplex PCR

| | |
|---|---|
| Product | concentration |

| | | | |
|---|---|---|---|
| MAPH | Ag | size | of primer |

| | | | | | |
|---|---|---|---|---|---|
| PCR-primer | system | Sequence | Tm* | (bp) | in mpx (nM) |

| | | | | | |
|---|---|---|---|---|---|
| HPA1-left | HPA-1 | gccgcgaattcactagtgcttcaggtcacagcgaggt | 58.98 | | 5 |
| HPA1-right | HPA-1 | ggccgcgggaattcgattgctccaatgtacggggtaaa | 59.82 | 185 | 5 |
| HPA2-left | HPA-2 | gccgcgaattcactagtgtgaaaggcaatgagctgaag | 59.15 | | 5 |
| HPA2-right | HPA-2 | ggccgcgggaattcgattagccagactgagcttctcca | 60.28 | 113 | 5 |
| HPA3-left | HPA-3 | gccgcgaattcactagtggcctgaccactcctttgc | 59.35 | | 7.5 |
| HPA3-right | HPA-3 | ggccgcgggaattcgattggaagatctgtctgcgatcc | 59.77 | 141 | 7.5 |
| HPA4-left | HPA-4 | gccgcgaattcactagtgatccgcaggttactggtgag | 60.13 | | 5 |
| HPA4-right | HPA-4 | ggccgcgggaattcgattccatgaaggatgatctgtgg | 58.88 | 121 | 5 |
| HPA5-left | HPA-5 | gccgcgaattcactagtgtgcaaatgcaagttaaattaccag | 59.47 | | 5 |
| HPA5-right | HPA-5 | ggccgcgggaattcgattacagacgtgctcttggtaggt | 58.88 | 190 | 5 |
| Gov-left | Gov | gccgcgaattcactagtgtgtatcagttcttggttttgtgatg | 60.31 | | 5 |
| Gov-right | Gov | ggccgcgggaattcgattaaaaccagtagccacccaag | 59.10 | 156 | 5 |
| JK1/2-left | Kidd | gccgcgaattcactagtggtctttcagccccatttgag | 59.67 | | 10 |
| JK1/2-right | Kidd | ggccgcgggaattcgattgttgaaaccccagagtccaa | 59.94 | 86 | 10 |
| FY1/2-left | Duffy | gccgcgaattcactagtggaattcttcctatggtgtgaatga | 59.38 | | 5 |
| FY1/2-right | Duffy | ggccgcgggaattcgattaagaagggcagtgcagagtc | 59.60 | 153 | 5 |
| GATAbox-left | Duffy | gccgcgaattcactagtgggccctcattagtccttgg | 59.49 | | 5 |
| GATAbox-right | Duffy | ggccgcgggaattcgattgaaatgaggggcatagggata | 60.12 | 200 | 5 |
| KEL1/2-left | Kell | gccgcgaattcactagtgaagggaaatggccatactga | 59.39 | | 5 |
| KEL1/2-right | Kell | ggccgcgggaattcgattagctgtgtaagagccgatcc | 59.46 | 207 | 5 |
| KEL3/4-left | Kell | gccgcgaattcactagtggcctcagaaactggaacagc | 60.00 | | 10 |
| KEL3/4-right | Kell | ggccgcgggaattcgattagcaaggtgcaagaacactct | 59.15 | 103 | 10 |
| RHCEex2for | Rhesus | gccgcgaattcactagtgcgtctgcttccccctcc | 61.78 | | 5 |
| RHex2rev | Rhesus | ggccgcgggaattcgattctgaacagtgtgatgaccacc | 59.01 | 268 | 5 |
| RHCEex5-left | Rhesus | gccgcgaattcactagtgggatgttctggccaagtg | 57.47 | | 5 |
| RHex5rev | Rhesus | ggccgcgggaattcgattggctgtcaccacactgactg | 60.37 | 143 | 5 |
| RHDΨ-left | Rhesus | ggccgcgggaattcgattgtagtgagctggcccatca | 59.80 | | 5 |
| RHDΨ-right | Rhesus | gccgcgaattcactagtgtgtctagtttcttaccggcaagt | 59.38 | 380 | 5 |
| RHD-leftB | Rhesus | gccgcgaattcactagtgttataataacacttgtccacaggg | 57.25 | | 5 |
| RHD-rightC | Rhesus | ggccgcgggaattcgattcggctccgacggtatc | 58.61 | 182 | 5 |
| BigC-left | Rhesus | gccgcgaattcactagtgggccaccaccatttgaa | 58.77 | | 15 |
| BigC-rightintron2 | Rhesus | ggccgcgggaattcgattccatgaacatgccacttcac | 59.97 | 393 | 15 |
| MN-left | MNS | gccgcgaattcactagtgtgagggaatttgtcttttgca | 60.60 | | 5 |
| MN-right | MNS | ggccgcgggaattcgattcagaggcaagaattcctcca | 60.33 | 394 | 5 |
| U-left | MNS | gccgcgaattcactagtgcgctgatgttatctgtcttatttttc | 59.64 | | 5 |
| U-right | MNS | ggccgcgggaattcgattgatcgttccaataataccagcc | 59.71 | 175 | 5 |
| JO-left | Dombrock | gccgcgaattcactagtgcctggcttaaccaaggaaaa | 59.20 | | 5 |
| JO-right | Dombrock | ggccgcgggaattcgatttcatactgctgtggagtcctg | 58.89 | 181 | 5 |
| MAPH-reverse# | | gccgcgaattcactagtg | 57.94 | | 200 |
| MAPH-forward# | | ggccgcgggaattcgatt | 67.55 | | 200 |

*Tm values of chimeric primers are without MAPH-tags.
#Cy5 labelled at 5′-end
The gene specific part of the RHDΨ-right and the RHD-rightC primer has been published (Maaskant-van Wijk et al. Transfusion 39, 546, 1999). Published primer sequences Rex6AD3: 5′-TGTCTAGTTTCTTACCGGCAAGT-3′; R1068: 5′-ATTGCCGGCTCCGACGGTATC-3′)
The gene specific part of BigC-left has been published as part of a published primer and part of the bigC-rightintron2, RHex2rev, RHCEex5for primers have been published (Tax et al. Transfusion 42, 634-44, 2002).
Published primer sequences
R348: 5′-CTGAACAGTGTGATGACC-3′;
c/rev: 5′-TGATGACCACCTTCCCAGG-3′;
R637: 5′-GCCCTCTTCTTGTGGATG-3′;
C/for: 5′-CAGCGCCACCACCATTTGAA-3′;
C/rev: 5′-GAACATCCCACTTCACTCCAG-3′)
Part of the MN-primers has been published (Akane et al. Vox Sang, 79, 183-7, 2000)
Published primer sequences MN-Left: 5′-GAGGGAATTTGTCTTTTGCA-3′ and MN-Right: 5′-AGAGGCAAGAATTCCTCC-3′.

TABLE III
Scorings criteria for genotyping HPA-1

| ID | F_{med} | Fₜₕ | F_{med}− F_{bg} | pos(F_{med}− F_{bg}) | ratio | genotype | | | |
|---|---|---|---|---|---|---|---|---|---|
| C a33 | 861 | | | | | | av. C | 692 | |
| C a35 | 579 | | | | | | SD | 105 | |
| C a38 | 688 | | | | | | 3*SD | 315 | |
| C a42 | 640 | | | | | | | aa > | 0.75 |
| C a43 | 692 | | | | | | | bb < | 0.25 |
| CS05 | 64433 | | | | | | | 0.35 < ab < | 0.65 |
| HPA-1aa | 1311 | 304.2 | 619 | 619 | 0.0 | bb | | | |
| HPA-1aa CR | 680 | −327 | −12 | 1 | 0.0 | bb | | | |
| HPA-1ab | 911 | −95.8 | 219 | 219 | 0.0 | bb | | | |
| HPA-1ab CR | 613 | [symbol] | −79 | 1 | 0.0 | [symbol] | | | |
| HPA-1ac | 7100 | 6093 | 6408 | 6408 | 0.2 | bb | | | |
| HPA-1ac CR | 884 | −123 | 192 | 192 | 0.2 | bb | | | |
| HPA-1ad | 7319 | 6312 | 6627 | 6627 | 0.2 | bb | | | |
| HPA-1ad CR | 614 | −393 | −78 | 1 | 0.0 | bb | | | |
| HPA-1ae | 39718 | 38711 | 39026 | 39026 | 0.4 | ab | | | |
| HPA-1ae CR | 792 | −215 | 100 | 100 | 0.2 | bb | | | |
| HPA-1ba | 29825 | 28818 | 29133 | 29133 | | | | | |
| HPA-1ba CR | 1159 | 152.2 | 467 | 467 | | | | | |
| HPA-1bb | 10446 | 9439 | 9754 | 9754 | | | | | |
| HPA-1bb CR | 827 | [symbol] | 135 | 135 | | | | | |
| HPA-1bc | 26518 | 25511 | 25826 | 25826 | | | | | |
| HPA-1bc CR | 1548 | 541.2 | 856 | 856 | | | | | |
| HPA-1bd | 30718 | 29711 | 30026 | 30026 | | | | | |
| HPA-1bd CR | 1276 | 269.2 | 584 | 584 | | | | | |
| HPA-1be | 65375 | 64368 | 64683 | 64683 | | | | | |
| HPA-1be CR | 1060 | 53.18 | 368 | 368 | | | | | |

Most frequent genotype results of a sample are in bold. Excluded genotype result bold and boxed.

TABLE IV
Genotyping results of 9 samples of the blind panel

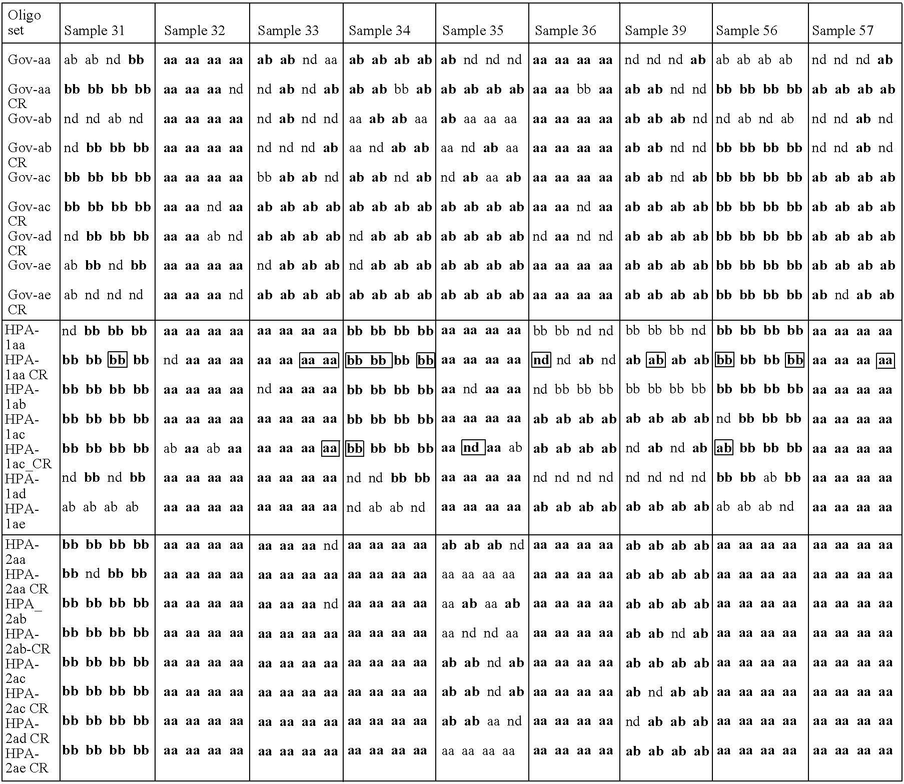

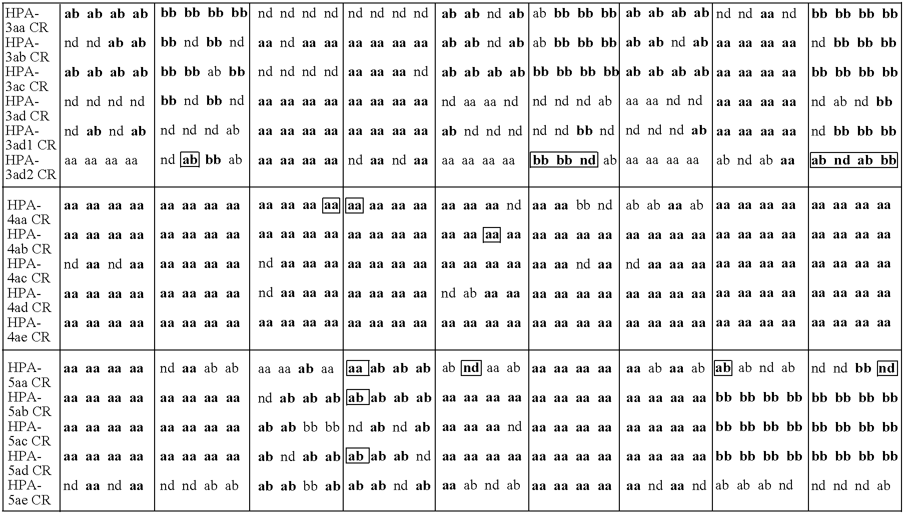

Most frequent genotype results of a sample are in bold. Excluded genotype results are in bold and boxed.

TABLE V
Genotypes of blind panel of 58 samples

| | HPA- | % pos | | | % pos | | | % pos | | | % pos | | | % pos | | | | |
|---|---|---|---|---|---|---|---|---|---|---|---|---|---|---|---|---|---|---|
| Sample | 1 | HPA-1 | | HPA-2 | HPA-2 | | HPA-3 | HPA-3 | | HPA-4 | HPA-4 | | HPA-5 | HPA-5 | | Gov | % pos gov | |
| 1 | aa | 100% | 28/28 | aa | 94% | 30/32 | bb | 54% | 13/24 | aa | 85% | 17/20 | aa | 75% | 15/20 | ab | 75% | 27/36 |
| 2 | aa | 96% | 24/25 | aa | 100% | 32/32 | aa | 83% | 20/24 | aa | 100% | 20/20 | aa | 79% | 15/19 | ab | 64% | 23/36 |
| 3 | bb | 70% | 19/27 | aa | 100% | 31/31 | ab | 50% | 12/24 | aa | 84% | 16/19 | ab | 80% | 16/20 | bb | 72% | 26/36 |
| 4 | ab | 43% | 10/23 | aa | 100% | 32/32 | aa | 79% | 19/24 | aa | 94% | 17/18 | ab | 45% | 5/11 | aa | 67% | 24/36 |
| 5 | aa | 96% | 27/28 | aa | 100% | 32/32 | aa | 83% | 20/24 | aa | 100% | 20/20 | aa | 68% | 13/19 | aa | 94% | 34/36 |
| 6 | aa | 93% | 25/27 | ab | 69% | 22/32 | ab | 42% | 10/24 | aa | 100% | 20/20 | ab | 53% | 10/19 | aa | 58% | 21/36 |
| 7 | ab | 54% | 15/28 | ab | 81% | 26/32 | aa | 79% | 19/24 | aa | 100% | 20/20 | ab | 60% | 12/20 | ab | 72% | 26/36 |
| 8 | aa | 96% | 27/28 | aa | 100% | 32/32 | aa | 83% | 20/24 | aa | 95% | 19/20 | bb | 65% | 13/20 | ab | 81% | 29/36 |
| 9 | bb | 81% | 21/26 | aa | 100% | 32/32 | ab | 38% | 9/24 | aa | 90% | 18/20 | ab | 65% | 13/20 | ab | 78% | 28/36 |
| 10 | aa | 96% | 27/28 | ab | 72% | 23/32 | aa | 79% | 19/24 | aa | 95% | 19/20 | aa | 85% | 17/20 | ab | 83% | 30/36 |
| 11 | bb | 70% | 16/23 | aa | 97% | 30/31 | bb | 44% | 7/16 | aa | 93% | 13/14 | bb | 81% | 13/16 | aa | 83% | 30/36 |
| 12 | aa | 100% | 23/23 | ab | 55% | 17/31 | ab | 46% | 11/24 | aa | 83% | 10/12 | aa | 80% | 16/20 | bb | 75% | 27/36 |
| 13 | ab | 54% | 15/28 | ab | 63% | 20/32 | ab | 50% | 12/24 | aa | 85% | 17/20 | ab | 65% | 13/20 | aa | 64% | 23/36 |
| 14 | ab | 44% | 12/27 | bb | 94% | 30/32 | ab | 50% | 12/24 | aa | 100% | 20/20 | aa | 85% | 17/20 | bb | 64% | 23/36 |
| 15 | aa | 93% | 26/28 | aa | 100% | 32/32 | ab | 38% | 9/24 | aa | 90% | 18/20 | ab | 55% | 11/20 | aa | 72% | 26/36 |
| 16 | aa | 96% | 27/28 | aa | 75% | 24/32 | ab | 42% | 10/24 | aa | 89% | 17/19 | ab | 70% | 14/20 | ab | 81% | 29/36 |
| 17 | bb | 74% | 20/27 | ab | 53% | 17/32 | ab | 39% | 9/23 | aa | 95% | 19/20 | aa | 78% | 14/18 | ab | 67% | 24/36 |
| 18 | aa | 93% | 25/27 | ab | 81% | 26/32 | ab | 33% | 8/24 | aa | 75% | 15/20 | ab | 75% | 15/20 | aa | 94% | 34/36 |
| 19 | aa | 96% | 27/28 | ab | 78% | 25/32 | ab | 54% | 13/24 | aa | 100% | 20/20 | aa | 65% | 13/20 | ab | 69% | 25/36 |
| 20 | bb | 75% | 21/28 | aa | 97% | 31/32 | aa | 83% | 20/24 | aa | 100% | 20/20 | aa | 90% | 18/20 | ab | 64% | 23/36 |
| 21 | aa | 100% | 28/28 | ab | 66% | 21/32 | bb | 61% | 14/23 | aa | 95% | 19/20 | aa | 100% | 20/20 | bb | 61% | 22/36 |
| 22 | aa | 96% | 26/27 | aa | 100% | 32/32 | aa | 88% | 21/24 | aa | 90% | 18/20 | aa | 80% | 16/20 | bb | 83% | 30/36 |
| 23 | aa | 100% | 28/28 | aa | 100% | 29/29 | aa | 88% | 21/24 | aa | 85% | 17/20 | aa | 80% | 16/20 | bb | 78% | 28/36 |
| 24 | aa | 100% | 28/28 | aa | 100% | 31/31 | ab | 42% | 10/24 | aa | 100% | 20/20 | ab | 65% | 13/20 | bb | 53% | 19/36 |
| 25 | bb | 85% | 22/26 | ab | 78% | 25/32 | ab | 38% | 9/24 | aa | 72% | 13/18 | aa | 79% | 15/19 | aa | 94% | 34/36 |
| 26 | aa | 96% | 27/28 | aa | 100% | 32/32 | aa | 79% | 19/24 | aa | 95% | 18/19 | ab | 95% | 18/19 | ab | 64% | 23/36 |
| 27 | aa | 81% | 21/26 | aa | 100% | 31/31 | aa | 63% | 15/24 | aa | 86% | 12/14 | aa | 100% | 19/19 | ab | 64% | 23/36 |
| 28 | aa | 79% | 22/28 | aa | 100% | 32/32 | bb | 68% | 16/22 | aa | 80% | 16/20 | aa | 75% | 15/20 | ab | 72% | 26/36 |
| 29 | aa | 100% | 28/28 | ab | 69% | 22/32 | ab | 54% | 13/24 | aa | 84% | 16/19 | aa | 80% | 16/20 | ab | 67% | 24/36 |
| 30 | bb | 70% | 16/23 | aa | 100% | 32/32 | bb | 72% | 13/18 | aa | 85% | 11/13 | aa | 94% | 15/16 | ab | 54% | 19/35 |
| 31 | bb | 74% | 20/27 | bb | 97% | 31/32 | ab | 50% | 12/24 | aa | 90% | 18/20 | aa | 90% | 18/20 | bb | 58% | 21/36 |
| 32 | aa | 89% | 25/28 | aa | 100% | 32/32 | bb | 52% | 12/23 | aa | 100% | 20/20 | aa | 65% | 13/20 | aa | 86% | 31/36 |
| 33 | aa | 96% | 24/25 | aa | 94% | 30/32 | aa | 63% | 15/24 | aa | 94% | 17/18 | ab | 60% | 12/20 | ab | 64% | 23/36 |
| 34 | bb | 75% | 18/24 | aa | 100% | 32/32 | aa | 71% | 17/24 | aa | 100% | 19/19 | ab | 76% | 13/17 | ab | 78% | 28/36 |
| 35 | aa | 93% | 25/27 | ab | 41% | 13/32 | ab | 46% | 11/24 | aa | 84% | 16/19 | aa | 68% | 13/19 | ab | 69% | 25/36 |
| 36 | ab | 48% | 13/27 | aa | 100% | 32/32 | bb | 52% | 11/21 | aa | 85% | 17/20 | aa | 100% | 20/20 | aa | 86% | 31/36 |
| 37 | aa | 100% | 28/28 | ab | 72% | 23/32 | bb | 63% | 15/24 | aa | 100% | 19/19 | aa | 100% | 20/20 | ab | 78% | 28/36 |
| 38 | aa | 96% | 27/28 | ab | 78% | 25/32 | ab | 46% | 11/24 | aa | 100% | 20/20 | aa | 85% | 17/20 | ab | 78% | 28/36 |
| 39 | ab | 48% | 13/27 | ab | 91% | 29/32 | ab | 50% | 12/24 | aa | 80% | 16/20 | aa | 80% | 16/20 | ab | 75% | 27/36 |
| 40 | ab | 44% | 12/27 | aa | 100% | 32/32 | bb | 64% | 14/22 | aa | 84% | 16/19 | ab | 65% | 13/20 | aa | 86% | 31/36 |
| 41 | ab | 56% | 15/27 | aa | 100% | 32/32 | ab | 54% | 13/24 | aa | 100% | 19/19 | aa | 65% | 11/17 | ab | 75% | 27/36 |
| 42 | aa | 100% | 28/28 | ab | 59% | 19/32 | ab | 54% | 13/24 | aa | 90% | 18/20 | aa | 100% | 20/20 | bb | 67% | 24/36 |
| 43 | aa | 96% | 27/28 | ab | 72% | 23/32 | aa | 79% | 19/24 | aa | 100% | 20/20 | aa | 100% | 20/20 | bb | 53% | 19/36 |
| 44 | aa | 100% | 28/28 | aa | 100% | 32/32 | bb | 71% | 17/24 | aa | 95% | 19/20 | aa | 90% | 18/20 | bb | 53% | 19/36 |
| 45 | aa | 100% | 28/28 | ab | 63% | 20/32 | bb | 68% | 15/22 | aa | 100% | 20/20 | aa | 95% | 19/20 | bb | 56% | 20/36 |
| 46 | ab | 35% | 7/20 | aa | 94% | 30/32 | ab | 35% | 8/23 | aa | 79% | 11/14 | aa | 83% | 15/18 | ab | 61% | 22/36 |
| 47 | aa | 81% | 21/26 | ab | 78% | 25/32 | ab | 50% | 12/24 | aa | 71% | 12/17 | aa | 73% | 11/15 | ab | 69% | 24/35 |
| 48 | aa | 93% | 25/27 | ab | 94% | 30/32 | ab | 42% | 10/24 | aa | 84% | 16/19 | aa | 80% | 16/20 | ab | 64% | 23/36 |
| 49 | bb | 70% | 19/27 | aa | 100% | 32/32 | ab | 67% | 16/24 | aa | 100% | 20/20 | aa | 63% | 10/16 | bb | 64% | 23/36 |
| 50 | aa | 93% | 26/28 | aa | 100% | 32/32 | ab | 46% | 11/24 | aa | 95% | 19/20 | aa | 80% | 16/20 | ab | 72% | 26/36 |
| 51 | aa | 96% | 27/28 | ab | 72% | 23/32 | aa | 83% | 20/24 | aa | 85% | 17/20 | aa | 75% | 15/20 | ab | 61% | 22/36 |
| 52 | aa | 100% | 22/22 | aa | 100% | 32/32 | aa | 79% | 19/24 | aa | 89% | 16/18 | aa | 84% | 16/19 | bb | 75% | 27/36 |
| 53 | bb | 71% | 17/24 | aa | 100% | 32/32 | bb | 64% | 14/22 | aa | 94% | 15/16 | aa | 78% | 14/18 | bb | 69% | 25/36 |
| 54 | aa | 89% | 24/27 | aa | 100% | 32/32 | ab | 46% | 11/24 | aa | 83% | 15/18 | ab | 67% | 12/18 | ab | 67% | 24/36 |
| 55 | aa | 100% | 27/27 | aa | 100% | 32/32 | ab | 50% | 12/24 | aa | 100% | 19/19 | aa | 90% | 18/20 | aa | 83% | 30/36 |
| 56 | bb | 76% | 19/25 | aa | 100% | 32/32 | aa | 75% | 18/24 | aa | 100% | 20/20 | bb | 63% | 12/19 | bb | 78% | 28/36 |
| 57 | aa | 100% | 27/27 | aa | 100% | 32/32 | bb | 80% | 16/20 | aa | 100% | 20/20 | bb | 68% | 13/19 | ab | 72% | 26/36 |
| 58 | ab | 50% | 14/28 | bb | 100% | 32/32 | ab | 50% | 12/24 | aa | 70% | 14/20 | aa | 75% | 15/20 | bb | 83% | 30/36 |

Genotypes are in bold when the predicted genotype is higher then 50% for the homozygote genotypes or 40% for the heterozygote genotypes (HPA-3ab >30%)

TABLE VI
HPA-3 genotyping results of sample 9 compared with the results of 6 other samples

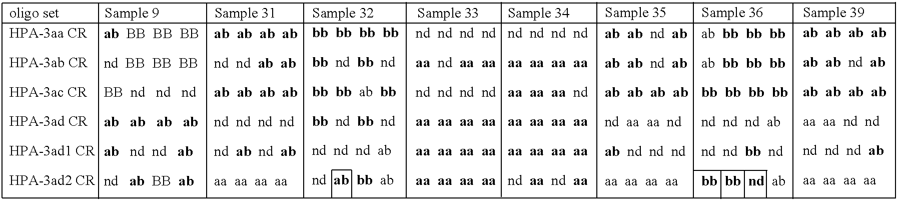

Most frequent genotype results of a sample are in bold. Excluded genotypes are in bold and boxed. Genotype of sample 9 should be HPA-3bb and therefore the bb-genotypes are shown in capital letters for this sample.

TABLE VII

Additionally tested primers for multiplex PCR
These primers have been tested together with primers of SEQ ID NOS:15 to 40 in
one multiplex PCR. PCR products of the correct product size were obtained.

Product

| | |
|---|---|
| Ag | size |

| | | | | |
|---|---|---|---|---|
| system | MAPH PCR-primer | Sequence | Tm* | (bp) |

| | | | | |
|---|---|---|---|---|
| Duffy | Fyx-left (MAPH) | gccgcgaattcactagtgTCATGCTTTTCAGACCTCTCTTC | 60.01 | |
| Duffy | Fyx-right (MAPH2) | ggccgcgggaattcgattCAAGACGGGCACCACAAT | 60.53 | 143 |
| Kell | KEL6/7-left (MAPH) | gccgcgaattcactagtgGCAGCACCAACCCTATGTTC | 60.53 | |
| Kell | KEL6/7-right (MAPH) | ggccgcgggaattcgattTCAGGCACAGGTGAGCTTC | 60.13 | 154 |
| Rhesus | RHDex3-left (MAPH) | gccgcgaattcactagtgTCCTGGCTCTCCCTCTCT | 57.48 | |
| Rhesus | RHCEex3-right (MAPH) | ggccgcgggaattcgattTTTTTCAAAACCCCGGAAG | 59.90 | 294 |
| Rhesus | RhDVI-left (MAPH) (fw) | ggccgcgggaattcgattCTTTGAATTAAGCACTTCACAGA | 56.48 | |
| Rhesus | RhDVI-right (MAPH) (rev) | gccgcgaattcactagtgGCCAGAATCACACTCCTGCT | 60.42 | 277 |
| MNS | Ss-left (MAPH3) | gccgcgaattcactagtgTTTTTCTTTGCACATGTCTTT | 55.20 | |
| MNS | Ss-right (MAPH2) | ggccgcgggaattcgattTCTTTGTCTTTACAATTTCGTGTG | 58.42 | 276 |
| Dombrock | DO-left (MAPH) | ggccgcgggaattcgattTGATCCCTCCCTATGAGCTG | 60.17 | |
| Dombrock | DO-right (MAPH) | gccgcgaattcactagtgTTATATGTGCTCAGGTTCCCAGT | 59.91 | 136 |
| Colton | Colton-left (MAPH) | gccgcgaattcactagtgGCCACGACCCTCTTTGTCT | 60.25 | |
| Colton | Colton-right (MAPH) | ggccgcgggaattcgattTACATGAGGGCACGGAAGAT | 60.48 | 269 |
| Diego | Diego-left (MAPH) | gccgcgaattcactagtgACTTATTCACGGGCATCCAG | 59.96 | |
| Diego | Diego-right (MAPH) | ggccgcgggaattcgattAAGCTCCACGTTCCTGAAGA | 59.99 | 185 |
| Diego | Wr-left (MAPH) | gccgcgaattcactagtgGGCTTCAAGGTGTCCAACTC | 59.70 | |
| Diego | Wr-right (MAPH) | ggccgcgggaattcgattAGGATGAAGACCAGCAGAGC | 59.56 | 158 |
| Yt | Yt-left (MAPH) | ggccgcgggaattcgattCCTTCGTGCCTGTGGTAGAT | 60.13 | |
| Yt | Yt-right (MAPH) | gccgcgaattcactagtgTTCTGGGACTTCTGGGAATG | 60.04 | 235 |
| Lutheran | Lu-left (MAPH) | gccgcgaattcactagtgGGACCCAGAGAGAGAGAGACTG | 59.61 | |
| Lutheran | Lu-right (MAPH) | ggccgcgggaattcgattGGGAGTCCAGCTGGTATGG | 60.48 | 220 |

*Tm values are without MAPH tags.

### References

Akane A, Mizukami H and Shiono H. 2000. Classification of standard alleles of the MN blood group system. Vox Sang 79, 183-187.
Belgrader P, Marino M M, Lubin M and Barany F. 1996. A Multiplex PCR-Ligase Detection Reaction Assay for Human Identity Testing. Genome Sci. Technol. 1, 77-87.
Berry J E, Murphy C M, Smith G A, Ranasinghe E, Finberg R, Walton J, Brown J, Navarrete C, Metcalfe P and Ouwehand W H. 2000. Detection of Gov system antibodies by MAIPA reveals an immunogenicity similar to the HPA-5 alloantigens. Br J Haematol. 110, 735-742.
Beuningen R van, van Damme H, Boender P, Bastiaensen N, Chan A and Kievits T. 2001. Fast and specific hybridisation using flow-through microarrays on porous metal oxide. Clin. Chem. 47, 1931-1933.
Brownie J, Shawcross S, Theaker J, Whitcombe D, Ferrie R, Newton C and Little S. 1997. The elimination of primer-dimer accumulation in PCR. Nucl. Acids Res. 25, 3235-3241.
Chamberlain J S, Gibbs R A, Ranier J E, Nguyen P N, and Caskey C T. 1988. Deletion screening of the Duchenne muscular dystrophy locus via multiplex DNA amplification. Nucl. Acids Res. 16, 11141-11156.
Cheek B J, Steel A B, Torres M P, Yu Y Y and Yang H. 2001. Chemiluminescence detection for hybridization assays on the flow-thru chip, a three-dimensional microchannel biochip. Anal Chem. 73, 5777-5783.
Evans J G and Lee-Tataseo C. 2002. Determination of the factor V Leiden single-nucleotide polymorphism in a commercial clinical laboratory by use of NanoChip microelectronic array technology. Clin Chem. 48, 1406-1411.
Fan J B, Chen X, Halushka M K, Bemo A, Huang X, Ryder T, Lipshutz R J, Lockhart D J and Chakravarti A. 2000. Parallel genotyping of human SNPs using generic high-density oligonucleotide tag arrays. Genome Res. 10, 853-860.
Fodor S P, Read J L, Pirrung M C, Stryer L, Lu A T and Solas D. 1991. Light-directed, spatially addressable parallel chemical synthesis. Science 251, 767-773.
Guo Z, Gatterman M S, Hood L, Hansen J A and Petersdorf E W. 2001. Oligonucleotide arrays for high-throughput SNPs detection in the MHC class I genes: HLA-B as a model system. Genome Res. 12, 447-457.
Hacia J G, Brody L C, Chee M S, Fodor S P and Collins F S. 1996. Detection of heterozygous mutations in BRCA1 using high density oligonucleotide arrays and two-colour fluorescence analysis. Nat Genet. 14, 441-447.
Heath K E, Day I N M and Humphries S E. 2000. Universal primer quantitative fluorescent multiplex (UPQFM) PCR: a method to detect major and minor rearrangements of the low density lipoprotein receptor gene. J. Med. Genet. 37, 272-280.
Hinds D A, Stokowski R P, Patil N, Konvicka K, Kershenobich D, Cox D R and Ballinger D G. 2004. Matching strategies for genetic association studies in structured populations. Am J Hum Genet. 74, 317-325.
Huang J X, Mehrens D, Wiese R, Lee S, Tam S W, Daniel S, Gilmore J, Shi M and Lashkari D. 2001. High-throughput genomic and proteomic analysis using microarray technology. Clin Chem. 47, 1912-1916.
Iwasaki H, Ezura Y, Ishida R, Kajita M, Kodaira M, Knight J, Daniel S, Shi M and Emi M. 2002. Accuracy of genotyping for single nucleotide polymorphisms by a microarray-based single nucleotide polymorphism typing method involving hybridization of short allele-specific oligonucleotides. DNA Res. 9, 59-62.
Jobs M, Howell W M, Stromqvist L, Mayr T and Brookes A J. 2003. DASH-2: flexible, low-cost, and high-throughput SNP genotyping by dynamic allele-specific hybridization on membrane arrays. Genome Res. 13, 916-924.
Kajiyama T, Miyahara Y, Iricka L J, Wilding P, Graves D J, Surrey S and Fortina P. 2003. Genotyping on a thermal gradient DNA chip. Genome Res. 13, 467-475.
Lindroos K, Sigurdsson S, Johansson K, Ronnblom L and Syvanen A C. 2002. Multiplex SNP genotyping in pooled DNA samples by a four-colour microarray system. Nucleic Acids Res. 30, e70.
Lu M, Shortreed M R, Hall J G, Wang L, Berggren T, Stevens P W, Kelso D M, Lyamichev V, Neri B and Smith L M. 2002. A surface invasive cleavage assay for highly parallel SNP analysis. Hum. Mutat. 19, 416-422.
Maaskant-van Wijk P A, Faas B H, de Ruijter J A, Overbeeke M A, von dem Borne A E, van Rhenen D J and van der Schoot C E. 1999. Genotyping of RHD by multiplex polymerase chain reaction analysis of six RHD-specific exons. Transfusion 39, 546.
Metcalfe P, Watkins N A, Ouwehand W H, Kaplan C, Newman P, Kekomaki R, De Haas M, Aster R, Shibata Y, Smith J, Kiefel V and Santoso S. 2003. Nomenclature of human platelet antigens. Vox Sang. 85, 240-245.
Miller S A, Dykes D D and Polesky H F. 1988. A simple salting out procedure for extracting DNA from human nucleated cells. Nucl. Acids Res. 16, 1215.
Mujumdar R B, Ernst L A, Mujumdar S R, Lewis C J and Waggoner A S. 1993. Cyanine dye labeling reagents: sulfoindocyanine succinimidyl esters. Bioconjug. Chem. 4, 105-111.
Park S J, Taton T A and Mirkin C A. 2002. Array-based electrical detection of DNA with nanoparticle probes. Science 295, 1503-1506.
Pastinen T, Kurg A, Metspalu A, Peltonen L and Syvanen A C. 1997. Minisequencing: a specific tool for DNA analysis and diagnostics on oligonucleotide arrays. Genome Res. 7, 606-614.
Pastinen T, Raitio M, Lindroos K, Tainola P, Peltonen L and Syvanen A C. 2000. A system for specific, high-throughput genotyping by allele-specific primer extension on microarrays. Genome Res. 10, 1031-1042.
Prix L, Uciechowski P, Bockmann B, Giesing M and Schuetz A J. 2002. Diagnostic biochip array for fast and sensitive detection of K-ras mutations in stool. Clin. Chem. 48, 428-435.
Randen I, Sorensen K, Killie M K and Kjeldsen-Kragh J. 2003. Rapid and reliable genotyping of human platelet antigen (HPA)-1, -2, -3, -4, and -5 a/b and Gov a/b by melting curve analysis. Transfusion 43, 445-450.
Reid M E. 2003. Applications of DNA-based assays in blood group antigen and antibody identification. Transfusion 43, 1748-1757.
Seltsam A, Wagner F F, Salama A and Flegel W A. 2003. Antibodies to high-frequency antigens may decrease the quality of transfusion support: an observational study. Transfusion 43, 1563-1566.
Shuber A P, Grondin V J and Klinger K W. 1995. A simplified procedure for developing multiplex PCRs. Genome Research 5, 488-493.
Tax M G, van der Schoot C E, van Doom R, Douglas-Berger L, van Rhenen D J and Maaskant-vanwijk P A. 2002. RHC and RHc genotyping in different ethnic groups. Transfusion 42, 634-444.
Wang D G, Fan J B, Siao C J, Berno A, Young P, Sapolsky R, Ghandour G, Perkins N, Winchester E, Spencer J, Kruglyak L, Stein L, Hsie L, Topaloglou T, Hubbell E, Robinson E, Mittnmann M, Morris M S, Shen N, Kilbum D, Rioux J, Nusbaum C, Rozen S, Hudson T J and Lander E S. 1998. Large-Scale Identification, Mapping, and Genotyping of Single-Nucleotide Polymorphisms in the Human Genome. Science 280, 1077-1082.
Wen W H, Bernstein L, Lescallett J, Beazer-Barclay Y, Sullivan-Halley J, White M and Press M F. 2000. Comparison of TP53 mutations identified by oligonucleotide microarray and conventional DNA sequence analysis. Cancer Res. 60, 2716-2722.
Wen S Y, Wang H, Sun O J, Wang S Q. 2003. Rapid detection of the known SNPs of CYP2C9 using oligonucleotide microarray. World J Gastroenterol. 9, 1342-1346.
White S, Kalf M, Liu Q, Villerius M, Engelsma D, Kriek M, Vollebregt E, Bakker B, van Ommen G J, Breuning M H and den Dunnen J T. 2002. Comprehensive detection of genomic duplications and deletions in the DMD gene, by use of multiplex amplifiable probe hybridisation. Am. J. Hum. Genet. 71, 365-374.

1. A method to amplify and detectable label a region of the loci of at least two different blood cell antigens that contain a site of a nucleotide polymorphism of said blood cell antigen which method comprises
subjecting DNA from an individual of a mammalian species to a multiplex Polymerase Chain Reaction (PCR),
wherein said each PCR of said multiplex employs at least one pair of chimeric blood cell antigen-specific primers for each blood cell antigen to be genotyped and at least one detectably labeled universal primer,
wherein said at least one universal primer has a unique sequence not occurring in the DNA of said mammalian species, and
wherein each chimeric primer pair comprises a left chimeric primer and a right chimeric primer, each comprising a blood cell antigen-specific part at the 3′ end and a universal part at the 5′ end, wherein the nucleotide sequence of the universal part of the chimeric primers corresponds to the nucleotide sequence of said at least one universal primer, and wherein said blood cell antigen-specific parts of the chimeric primer pair enclose a region of the locus of the blood cell antigen which contains the site of a nucleotide polymorphism of said blood cell antigen.

2. The method of claim 1, wherein said at least one detectably labeled universal primer is used in a molar amount which is at least 100 times the molar amount of each chimeric primer.

3. The method of claim 1, wherein said each PCR employs a pair of detectably labeled universal primers each with a unique sequence not occurring in the DNA of said mammalian species, and
wherein for each chimeric primer pair the nucleotide sequence of the universal part of one member of the chimeric primer pair corresponds to the nucleotide sequence of one member of the universal primer pair and the nucleotide sequence of the universal part of the other member of the chimeric primer pair corresponds to the nucleotide sequence of the other member of the universal primer pair.

4. The method of claim 3, wherein one of said universal primers has the base sequence gcegcgaattcactagtg (SEQ ID NO:2) and the other universal primer has the base sequence ggccgcgggaattcgatt (SEQ ID NO:1).

5. The method of claim 1, wherein each universal primer carries a fluorescent label at its 5′ end.

6. The method of claim 1, wherein said mammalian species is human, said DNA is genomic DNA, and said blood cell antigens comprise at least two members selected from the group consisting of HPA1, HPA2, HPA3, HPA4, HPA5, Gov, JK1/2, FY1/2, GATAbox, KEL1/2, KEL3/4, RHCEex2/RHex2, RHCEex5/RHex5, RHDy, RHD, BigC, MN, U and JO.

7. The method of claim 6, wherein said chimeric primer pairs comprise at least two, pairs selected from the group consisting of:

| | | |
|---|---|---|
| HPA1-left | gccgcgaattcactagtgcttcaggtcacagcgaggt | SEQ ID NO:3 |
| HPA1-right | ggccgcgggaattcgattgctccaatgtacggggtaaa | SEQ ID NO:4 |
| HPA2-left | gccgcgaattcactagtgtgaaaggcaatgagctgaag | SEQ ID NO:5 |
| HPA2-right | ggccgcgggaattcgattagccagactgagcttctcca | SEQ ID NO:6 |
| HPA3-left | gccgcgaattcactagtggcctgaccactcctttgc | SEQ ID NO:7 |
| HPA3-right | ggccgcgggaattcgattggaagatctgtctgcgatcc | SEQ ID NO:8 |
| HPA4-left | gccgcgaattcactagtgatccgcaggttactggtgag | SEQ ID NO:9 |
| HPA4-right | ggccgcgggaattcgattccatgaaggatgatctgtgg | SEQ ID NO:10 |
| HPA5-left | gccgcgaattcactagtgtccaaatgcaagttaaattaccag | SEQ ID NO:11 |
| HPA5-right | ggccgcgggaattcgattacagacgtgctcttggtaggt | SEQ ID NO:12 |
| HPA15-left | gccgcgaattcactagtgtgtatcagttcttggttttgtgatg | SEQ ID NO:13 |
| HPA15-right | ggccgcgggaattcgattaaaaccagtagccacccaag | SEQ ID NO:14 |
| JK1/2-left | gccgcgaattcactagtggtctttcagccccatttgag | SEQ ID NO:15 |
| JK1/2-right | ggccgcgggaattcgattgttgaaaccccagagtccaa | SEQ ID NO:16 |
| FY1/2-left | gccgcgaattcactagtggaattcttcctatggtgtgaatga | SEQ ID NO:17 |
| FY1/2-right | ggccgcgggaattcgattaagaagggcagtgcagagtc | SEQ ID NO:18 |
| GATAbox-left | gccgcgaattcactagtgggccctcattagtccttgg | SEQ ID NO:19 |
| GATAbox-right | ggccgcgggaattcgattgaaatgaggggcatagggata | SEQ ID NO:20 |
| Fyx-left | gccgcgaattcactagtgtcatgcttttcagacctctcttc | SEQ ID NO:175 |
| Fyx-right | ggccgcgggaattcgattcaagacgggcaccacaat | SEQ ID NO:176 |
| KEL1/2-left | gccgcgaattcactagtgaagggaaatggccatactga | SEQ ID NO:21 |
| KEL1/2-right | ggccgcgggaattcgattagctgtgtaagagccgatcc | SEQ ID NO:22 |
| KEL3/4-left | gccgcgaattcactagtggcctcagaaactggaacagc | SEQ ID NO:23 |
| KEL3/4-right | ggccgcgggaattcgattagcaaggtgcaagaacactct | SEQ ID NO:24 |
| KEL6/7-left | gccgcgaattcactagtggcagcaccaaccctatgttc | SEQ ID NO:177 |
| KEL6/7-right | ggccgcgggaattcgatttcaggcacaggtgagcttc | SEQ ID NO:178 |
| RHCEex2for | gccgcgaattcactagtgcgtctgcttccccctcc | SEQ ID NO:25 |
| RHex2rev | ggccgcgggaattcgattctgaacagtgtgatgaccacc | SEQ ID NO:26 |
| RHDex3-left | gccgcgaattcactagtgtcctggctctccctctct | SEQ ID NO:179 |
| RHCEex3-right | ggccgcgggaattcgatttttttcaaaaccccggaag | SEQ ID NO:180 |
| RHCEex5-left | gccgcgaattcactagtgggatgttctggccaagtg | SEQ ID NO:27 |
| RHex5rev | ggccgcgggaattcgattggctgtcaccacactgactg | SEQ ID NO:28 |
| RHDΨ-left | ggccgcgggaattcgattgtagtgagctggcccatca | SEQ ID NO:29 |
| RHDΨ-right | gccgcgaattcactagtgtgtctagtttcttaccggcaagt | SEQ ID NO:30 |
| RHD-leftB | gccgcgaattcactagtgttataataacacttgtccacaggg | SEQ ID NO:31 |
| RHD-rightC | ggccgcgggaattcgattcggctccgacggtatc | SEQ ID NO:32 |
| BigC-left | gccgcgaattcactagtgggccaccaccatttgaa | SEQ ID NO:33 |
| BigC-rightintron2 | ggccgcgggaattcgattccatgaacatgccacttcac | SEQ ID NO:34 |
| RnDVI-left (fw) | ggccgcgggaattcgattctttgaattaagcacttcacaga | SEQ ID NO:181 |
| RhDVI-right (rev) | gccgcgaattcactagtggccagaatcacactcctgct | SEQ ID NO:182 |
| MN-left | gccgcgaattcactagtgtgagggaatttgtcttttgca | SEQ ID NO:35 |
| MN-right | ggccgcgggaattcgattcagaggcaagaattcctcca | SEQ ID NO:36 |
| Ss-left | gccgcgaattcactagtgtttttctttgcacatgtcttt | SEQ ID NO:183 |
| Ss-right | ggccgcgggaattcgatttctttgtctttacaatttcgtgtg | SEQ ID NO:184 |
| U-left | gccgcgaattcactagtgcgctgatgttatctgtcttatttttc | SEQ ID NO:37 |
| U-right | ggccgcgggaattcgattgatcgttccaataataccagcc | SEQ ID NO:38 |
| DO-left | ggccgcgggaattcgatttgatccctccctatgagctg | SEQ ID NO:185 |
| DO-right | gccgcgaattcactagtgttatatgtgctcaggttcccagt | SEQ ID NO:186 |
| JO-left | gccgcgaattcactagtgcctggcttaaccaaggaaaa | SEQ ID NO:39 |
| JO-right | ggccgcgggaattcgatttcatactgctgtggagtcctg | SEQ ID NO:40 |
| Colton-left | gccgcgaattcactagtggccacgaccctctttgtct | SEQ ID NO:187 |
| Colton-right | ggccgcgggaattcgatttacatgagggcacggaagat | SEQ ID NO:188 |
| Diego-left | gccgcgaattcactagtgacttattcacgggcatccag | SEQ ID NO:189 |
| Diego-right | ggccgcgggaattcgattaagctccacgttcctgaaga | SEQ ID NO:190 |
| Wr-left | gccgcgaattcactagtgggcttcaaggtgtccaactc | SEQ ID NO:636 |
| Wr-right | ggccgcgggaattcgattaggatgaagaccagcagagc | SEQ ID NO:637 |
| Yt-left | ggccgcgggaattcgattccttcgtgcctgtggtagat | SEQ ID NO:638 |
| Yt-right | gccgcgaattcactagtgttctgggacttctgggaatg | SEQ ID NO:639 |
| Lu-left | gccgcgaattcactagtgggacccagagagagagagactg | SEQ ID NO:640 |
| Lu-right | ggccgcgggaattcgattgggagtccagctggtatgg | SEQ ID NO:641 |

8. The method of claim 1, wherein the DNA polymerase used in the multiplex PCR is a heat-resistant polymerase and each cycle of the multiplex PCR comprises a heat denaturation step of 15 to 60 sec at a temperature of 90 to 98° C., an annealing step of 60 to 120 sec at a temperature of 54 to 60° C., and a primer extension step of 60 to 120 sec at a temperature of 68 to 76° C.

9. The method of claim 1, wherein said multiplex PCR uses, based on a reaction volume of 50 μl , about 100 ng of genomic DNA from said individual, about 5 nM of each chimeric primer, about 0.2 μl of each detectably labeled universal primer, about 25 μl of 2× MasterMix containing buffer, dNTP's and DNA polymerase.

10. The method of claim 1, which further comprises determining the genotype for each of said blood cell antigens by hybridizing the products of the multiplex PCR amplification, after denaturation, to blood cell antigen allele-specific oligonucleotide probes, and analyzing the hybridization pattern.

11. The method of claim 10, wherein the allele-specific probes have a length of 15 to 40 nucleotides.

12. The method of claim 10, wherein the probes comprise for each allele of a blood cell antigen at least two different sense probes and at least two different antisense probes, each of them covering the site of the nucleotide polymorphism but at varying positions.

13. The method of claim 10, wherein probes comprise at least 72 different probes selected from the group consisting of:

| HPA1-allele a: | | |
|---|---|---|
| HPA-1aa | tacaggccctgcctctggg | SEQ ID NO:41 |
| HPA-1ab | aggccctgcctctgggct | SEQ ID NO:42 |
| HPA-1ac | ccctgcctctgggctcacc | SEQ ID NO:43 |
| HPA-1ad | tgcctctgggctcacctcg | SEQ ID NO:44 |
| HPA-1ae | ctctgggctcacctcgctg | SEQ ID NO:45 |
| HPA-1aa CR | cagcgaggtgagcccagag | SEQ ID NO:46 |
| HPA-1ab CR | cgaggtgagcccagaggca | SEQ ID NO:47 |
| HPA-1ac CR | ggtgagcccagaggcaggg | SEQ ID NO:48 |
| HPA-1ad CR | agcccagaggcagggcct | SEQ ID NO:49 |
| HPA-1ae CR | cccagaggcagggcctgta | SEQ ID NO:50 |
| HPA1-allele b: | | |
| HPA-1ba | tacaggccctgcctccggg | SEQ ID NO:51 |
| HPA-1bb | aggccctgcctccgggct | SEQ ID NO:52 |
| HPA-1bc | ccctgcctccgggctcac | SEQ ID NO:53 |
| HPA-1bd | ctgcctccgggctcacct | SEQ ID NO:54 |
| HPA-1be | ctccgggctcacctcgct | SEQ ID NO:55 |
| HPA-1ba CR | agcgaggtgagcccggag | SEQ ID NO:56 |
| HPA-1bb CR | aggtgagcccggaggcag | SEQ ID NO:57 |
| HPA-1bc CR | gtgagcccggaggcaggg | SEQ ID NO:58 |
| HPA-1bd CR | agcccggaggcagggcct | SEQ ID NO:59 |
| HPA-1be CR | cccggaggcagggcctgta | SEQ ID NO:60 |
| HPA2-allele a: | | |
| HPA-2aa | ctgacgcccacacccaag | SEQ ID NO:61 |
| HPA-2ab | ctcctgacgcccacaccc | SEQ ID NO:62 |
| HPA-2ac | ggctcctgacgcccacac | SEQ ID NO:63 |
| HPA-2ad | agggctcctgacgcccac | SEQ ID NO:64 |
| HPA-2ae | ccagggctcctgacgccc | SEQ ID NO:65 |
| HPA-2aa CR | cttgggtgtgggcgtcag | SEQ ID NO:66 |
| HPA-2ab CR | gggtgtgggcgtcaggag | SEQ ID NO:67 |
| HPA-2ac CR | gtgtgggcgtcaggagcc | SEQ ID NO:68 |
| HPA-2ad CR | gtgggcgtcaggagccct | SEQ ID NO:69 |
| HPA-2ae CR | gggcgtcaggagccctgg | SEQ ID NO:70 |
| HPA2-allele b: | | |
| HPA-2ba | cctgatgcccacacccaag | SEQ ID NO:71 |
| HPA-2bb | ctcctgatgcccacaccca | SEQ ID NO:72 |
| HPA-2bc | ggctcctgatgcccacacc | SEQ ID NO:73 |
| HPA-2bd | agggctcctgatgcccaca | SEQ ID NO:74 |
| HPA-2be | ccagggctcctgatgccc | SEQ ID NO:75 |
| HPA-2ba CR | cttgggtgtgggcatcagg | SEQ ID NO:76 |
| HPA-2bb CR | tgggtgtgggcatcaggag | SEQ ID NO:77 |
| HPA-2bc CR | ggtgtgggcatcaggagcc | SEQ ID NO:78 |
| HPA-2bd CR | tgtgggcatcaggagccct | SEQ ID NO:79 |
| HPA-2be CR | gggcatcaggagccctgg | SEQ ID NO:80 |
| HPA3-allele a: | | |
| HPA-3aa | ccatccccagcccctccc | SEQ ID NO:81 |
| HPA-3ab | gcccatccccagcccctc | SEQ ID NO:82 |
| HPA-3ac | ctgcccatccccagcccc | SEQ ID NO:83 |
| HPA-3ad1 | gctgcccatccccagccc | SEQ ID NO:84 |
| HPA-3ad | ggctgcccatccccagcc | SEQ ID NO:85 |
| HPA-3ad2 | gggctgcccatccccagc | SEQ ID NO:86 |
| HPA-3ae | ggggctgcccatcccca | SEQ ID NO:87 |
| HPA-3aa CR | gggaggggctggggatgg | SEQ ID NO:88 |
| HPA-3ab CR | gaggggctggggatgggc | SEQ ID NO:89 |
| HPA-3ac CR | ggggctggggatgggcag | SEQ ID NO:90 |
| HPA-3ad1 CR | gggctggggatgggcagc | SEQ ID NO.91 |
| HPA-3ad CR | ggctggggatgggcagcc | SEQ ID NO:92 |
| HPA-3ad2 CR | gctggggatgggcagccc | SEQ ID NO:93 |
| HPA-3ae CR | tggggatgggcagcccc | SEQ ID NO:94 |
| HPA3-allele b: | | |
| HPA-3ba | ccagccccagcccctcc | SEQ ID NO:95 |
| HPA-3bb | gcccagccccagcccct | SEQ ID NO:96 |
| HPA-3bc | ctgcccagccccagccc | SEQ ID NO:97 |
| HPA-3bd1 | gctgcccagccccagcc | SEQ ID NO:98 |
| HPA-3bd | ggctgcccagccccagc | SEQ ID NO:99 |
| HPA-3bd2 | gggctgcccagccccag | SEQ ID NO:100 |
| HPA-3be | ggggctgcccagcccca | SEQ ID NO:101 |
| HPA-3ba CR | ggaggggctggggctgg | SEQ ID NO:102 |
| HPA-3bb CR | aggggctggggctgggc | SEQ ID NO:103 |
| HPA-3bc CR | gggctggggctgggcag | SEQ ID NO:104 |
| HPA-3bd1 CR | ggctggggctgggcagc | SEQ ID NO:105 |
| HPA-3bd CR | gctggggctgggcagcc | SEQ ID NO:106 |
| HPA-3bd2 CR | ctggggctgggcagccc | SEQ ID NO:107 |
| HPA-3be CR | tggggctgggcagcccc | SEQ ID NO:108 |
| HPA4-allele a: | | |
| HPA-4aa | gccacccagatgcgaaag | SEQ ID NO:109 |
| HPA-4ab | cacccagatgcgaaagct | SEQ ID NO:110 |
| HPA-4ac | cccagatgcgaaagctca | SEQ ID NO:111 |
| HPA-4ad | cagatgcgaaagctcacc | SEQ ID NO:112 |
| HPA-4ae | gatgcgaaagctcaccag | SEQ ID NO:113 |
| HPA-4aa CR | ctggtgagctttcgcatc | SEQ ID NO:114 |
| HPA-4ab CR | ggtgagctttcgcatctg | SEQ ID NO:115 |
| HPA-4ac CR | tgagctttcgcatctggg | SEQ ID NO:116 |
| HPA-4ad CR | agctttcgcatctgggtg | SEQ ID NO:117 |
| HPA-4ae CR | ctttcgcatctgggtggc | SEQ ID NO:118 |
| HPA4-allele b: | | |
| HPA-4ba | gccacccagatgcaaaag | SEQ ID NO:119 |
| HPA-4bb | ccacccagatgcaaaagct | SEQ ID NO:120 |
| HPA-4bc | acccagatgcaaaagctcac | SEQ ID NO:121 |
| HPA-4bd | cagatgcaaaagctcacca | SEQ ID NO:122 |
| HPA-4ba CR | ttactggtgagcttttgcatc | SEQ ID NO:124 |
| HPA-4bb CR | tggtgagcttttgcatctg | SEQ ID NO:125 |
| HPA-4bc CR | gtgagcttttgcatctgggt | SEQ ID NO:126 |
| HPA-4bd CR | agcttttgcatctgggtgg | SEQ ID NO:127 |
| HPA-4be CR | cttttgcatctgggtggc | SEQ ID NO:128 |
| HPA5-allele a: | | |
| HPA-5aa | gagtctacctgtttactatcaaagagg | SEQ ID NO:129 |
| HPA-5ab | agtctacctgtttactatcaaagaggta | SEQ ID NO:130 |
| HPA-5ac | gtctacctgtttactatcaaagaggtaa | SEQ ID NO:131 |
| HPA-5ad | ctacctgtttactatcaaagaggtaaaa | SEQ ID NO:132 |
| HPA-5ae | acctgtttactatcaaagaggtaaaaa | SEQ ID NO:133 |
| HPA-5aa CR | tttttacctctttgatagtaaacaggt | SEQ ID NO:134 |
| HPA-5ab CR | ttttacctctttgatagtaaacaggtag | SEQ ID NO:135 |
| HPA-5ac CR | ttacctctttgatagtaaacaggtagac | SEQ ID NO:136 |
| HPA-5ad CR | tacctctttgatagtaaacaggtagact | SEQ ID NO:137 |
| HPA-5ae CR | cctctttgatagtaaacaggtagactc | SEQ ID NO:138 |
| HPA5-allele b: | | |
| HPA-5ba | gagtctacctgtttactatcaaaaagg | SEQ ID NO:139 |
| HPA-5bb | agtctacctgtttactatcaaaaaggta | SEQ ID NO:140 |
| HPA-5bc | gtctacctgtttactatcaaaaaggtaa | SEQ ID NO:141 |
| HPA-5ad | ctacctgtttactatcaaaaaggtaaaa | SEQ ID NO:142 |
| HPA-5be | acctgtttactatcaaaaaggtaaaaa | SEQ ID NO:143 |
| HPA-5ba CR | tttttacctttttgatagtaaacaggt | SEQ ID NO:144 |
| HPA-5bb CR | ttttacctttttgatagtaaacaggtag | SEQ ID NO:145 |
| HPA-5bc CR | ttacctttttgatagtaaacaggtagac | SEQ ID NO:146 |
| HPA-5bd CR | tacctttttgatagtaaacaggtagact | SEQ ID NO:147 |
| HPA-5be CR | cctttttgatagtaaacaggtagactc | SEQ ID NO:148 |
| HPA15-allele a: | | |
| Gov-aa | ttattatcttgacttcagttacaggattt | SEQ ID NO:149 |
| Gov-ab | tcttgacttcagttacaggatttacc | SEQ ID NO:150 |
| Gov-ac | tgacttcagttacaggatttaccaa | SEQ ID NO:151 |
| Gov-ad | cttcagttacaggatttaccaagaat | SEQ ID NO:152 |
| Gov-ae | cagttacaggatttaccaagaatttg | SEQ ID NO:153 |
| Gov-aa CR | caaattcttggtaaatcctgtaactg | SEQ ID NO:154 |
| Gov-ab CR | attcttggtaaatcctgtaactgaag | SEQ ID NO:155 |
| Gov-ac CR | tggtaaatcctgtaactgaagtcaa | SEQ ID NO:156 |
| Gov-ad CR | ggtaaatcctgtaactgaagtcaaga | SEQ ID NO:157 |
| Gov-ae CR | aaatcctgtaactgaagtcaagataataa | SEQ ID NO:158 |
| HPA15-allele b: | | |
| Gov-ba | tatcttgacttcagttccaggatt | SEQ ID NO:159 |
| Gov-bb | cttgacttcagttccaggatttac | SEQ ID NO:160 |
| Gov-bc | gacttcagttccaggatttacca | SEQ ID NO:161 |
| Gov-bd | ttcagttccaggatttaccaag | SEQ ID NO:162 |
| Gov-be | cagttccaggatttaccaagaatt | SEQ ID NO:163 |
| Gov-ba CR | aattcttggtaaatcctggaactg | SEQ ID NO:164 |
| Gov-bb CR | cnggtaaatcctggaactgaa | SEQ ID NO:165 |
| Gov-bc CR | ggtaaatcctggaactgaagtca | SEQ ID NO:166 |
| Gov-bd CR | gtaaatcctggaactgaagtcaag | SEQ ID NO:167 |
| Gov-be CR | aatcctggaactgaagtcaagata | SEQ ID NO:168 |
| Colton-a-allele: | | |
| Co.a.1 | aacaaccagacggcggt | SEQ ID NO:191 |
| Co.a.2 | accagacggcggtccag | SEQ ID NO:192 |
| Co.a.3 | cagacggcggtccagga | SEQ ID NO:193 |
| Co.a.4 | gacggcggtccaggacaa | SEQ ID NO:194 |
| Co.a.5 | cggcggtccaggacaac | SEQ ID NO:195 |
| Co.a.1.cr | gttgtcctggaccgccgt | SEQ ID NO:196 |
| Co.a.2.cr | tcctggaccgccgtctg | SEQ ID NO:197 |
| Co.a.3.cr | ctggaccgccgtctggt | SEQ ID NO:198 |
| Co.a.4.cr | ggaccgccgtctggttg | SEQ ID NO:199 |
| Co.a.5.cr | accgccgtctggttgtt | SEQ ID NO:200 |
| Colton-b-allele: | | |
| Co.b.1 | ggaacaaccagacggtggt | SEQ ID NO.201 |
| Co.b.2 | acaaccagacggtggtccag | SEQ ID NO:202 |
| Co.b.3 | accagacggtggtccagga | SEQ ID NO:203 |
| Co.b.4 | gacggtggtccaggacaacg | SEQ ID NO:204 |
| Co.b.5 | cggtggtccaggacaacg | SEQ ID NO:205 |
| Co.b.1.cr | cgttgtcctggaccaccgt | SEQ ID NO:206 |
| Co.b.2.cr | ttgtcctggaccaccgtctg | SEQ ID NO:207 |
| Co.b.3.cr | ctggaccaccgtctggttgt | SEQ ID NO:208 |
| Co.b.4.cr | ggaccaccgtctggttgttc | SEQ ID NO:209 |
| Co.b.5.cr | accaccgtctggttgttcc | SEQ ID NO:210 |
| Diego-a-allele: | | |
| Di.a.1 | gtgaagtccacgccggc | SEQ ID NO:211 |
| Di.a.2 | gaagtccacgccggcct | SEQ ID NO:212 |
| Di.a.3 | agtccacgccggcctcc | SEQ ID NO:213 |
| Di.a.4 | tccacgccggcctccct | SEQ ID NO:214 |
| Di.a.5 | acgccggcctccctggcc | SEQ ID NO:215 |
| Di.a.1.cr | gccagggaggccggcgt | SEQ ID NO:216 |
| Di.a.2.cr | agggaggccggcgtgga | SEQ ID NO:217 |
| Di.a.3.cr | ggaggccggcgtggact | SEQ ID NO:218 |
| Di.a.4.cr | aggccggcgtggacttc | SEQ ID NO:219 |
| Di.a.5.cr | ggccggcgtggacttca | SEQ ID NO:220 |
| Diego-b-allele: | | |
| Di.b.1 | ggtgaagtccacgctggc | SEQ ID NO:221 |
| Di.b.2 | gtgaagtccacgctggcct | SEQ ID NO:222 |
| Di.b.3 | tgaagtccacgctggcctcc | SEQ ID NO:223 |
| Di.b.4 | gaagtccacgctggcctccct | SEQ ID NO:224 |
| Di.b.5 | acgctggcctccctggccc | SEQ ID NO:225 |
| Di.b.1.cr | ggccagggaggccagcgt | SEQ ID NO:226 |
| Di.b.2.cr | cagggaggccagcgtgga | SEQ ID NO:227 |
| Di.b.3.cr | agggaggccagcgtggact | SEQ ID NO:228 |
| Di.b.4.cr | ggaggccagcgtggacttc | SEQ ID NO:229 |
| Di.b.5.cr | ggccagcgtggacttcacc | SEQ ID NO:230 |
| Diego Wr-a-allele: | | |
| Wr.a.1 | tgggcttgcgttccaagt | SEQ ID NO:231 |
| Wr.a.2 | ggcttgcgttccaagtttc | SEQ ID NO:232 |
| Wr.a.3 | ttgcgttccaagtttccca | SEQ ID NO:233 |
| Wr.a.4 | cgttccaagtttcccatct | SEQ ID NO:234 |
| Wr.a.5 | tccaagtttcccatctgga | SEQ ID NO:235 |
| Wr.a.1 CR | tccagatgggaaacttgga | SEQ ID NO:236 |
| Wr.a.2 CR | agatgggaaacttggaacg | SEQ ID NO:237 |
| Wr.a.3 CR | tgggaaacttggaacgcaa | SEQ ID NO:238 |
| Wr.a.4 CR | gaaacttggaacgcaagcc | SEQ ID NO:239 |
| Wr.a.5 CR | acttggaacgcaagccca | SEQ ID NO:240 |
| Diego Wr-b-allele: | | |
| Wr.b.1 | gggcttgcgttccgagtt | SEQ ID NO:241 |
| Wr.b.2 | gcttgcgttccgagtttc | SEQ ID NO:242 |
| Wr.b.3 | ttgcgttccgagtttccc | SEQ ID NO:243 |
| Wr.b.4 | cgttccgagtttcccatc | SEQ ID NO:244 |
| Wr.b.5 | tccgagtttcccatctgg | SEQ ID NO:245 |
| Wr.b.1 CR | ccagatgggaaactcgga | SEQ ID NO:246 |
| Wr.b.2 CR | gatgggaaactcggaacg | SEQ ID NO:247 |
| Wr.b.3 CR | gggaaactcggaacgcaa | SEQ ID NO:248 |
| Wr.b.4 CR | gaaactcggaacgcaagc | SEQ ID NO:249 |
| Wr.b.5 CR | aactcggaacgcaagccc | SEQ ID NO:250 |
| Dombrock-a-allele: | | |
| Do.a.1 | taccacccaagaggaaact | SEQ ID NO:251 |
| Do.a.2 | ccacccaagaggaaactgg | SEQ ID NO:252 |
| Do.a.3 | acccaagaggaaactggttg | SEQ ID NO:253 |
| Do.a.4 | caagaggaaactggttgca | SEQ ID NO:254 |
| Do.a.5 | aggaaactggttgcagttga | SEQ ID NO:255 |
| Do a 6 cr | ctcaactgcaaccagtttcc | SEQ ID NO:256 |
| Do a 7 cr | caactgcaaccagtttcctc | SEQ ID NO:257 |
| Do a 8 cr | tgcaaccagtttcctcttgg | SEQ ID NO:258 |
| Do a 9 cr | accagtttcctcttgggtgg | SEQ ID NO:259 |
| Do a 10 cr | cagtttcctcttgggtggta | SEQ ID NO:260 |
| Dombrock-b-allele: | | |
| Do.b.1 | taccacccaagaggagact | SEQ ID NO:261 |
| Do.b.2 | ccacccaagaggagactgg | SEQ ID NO:262 |
| Do.b.3 | acccaagaggagactggttg | SEQ ID NO:263 |
| Do.b.4 | caagaggagactggttgca | SEQ ID NO:264 |
| Do.b.5 | aggagactggttgcagttga | SEQ ID NO:265 |
| Do b 6 cr | ctcaactgcaaccagtctcc | SEQ ID NO:266 |
| Do b 7 cr | caactgcaaccagtctcctc | SEQ ID NO:267 |
| Do b 8 cr | tgcaaccagtctcctcttgg | SEQ ID NO:268 |
| Do b 9 cr | accagtctcctcttgggtgg | SEQ ID NO:269 |
| Do b 10 cr | cagtctcctcttgggtggt | SEQ ID NO:270 |
| Dombrock-Joseph(a) positive allele: | | |
| Jo.a.pos.1 | ccccagaacatgactaccac | SEQ ID NO:271 |
| Jo.a.pos.2 | ccagaacatgactaccacaca | SEQ ID NO:272 |
| Jo.a.pos.3 | agaacatgactaccacacacgc | SEQ ID NO:273 |
| Jo.a.pos.4 | catgactaccacacacgctgt | SEQ ID NO:274 |
| Jo.a.pos.5 | actaccacacacgctgtgg | SEQ ID NO:275 |
| Jo.a.pos.1.cr | gccacagcgtgtgtggtagt | SEQ ID NO:276 |
| Jo.a.pos.2.cr | acagcgtgtgtggtagtcatg | SEQ ID NO:277 |
| Jo.a.pos.3.cr | agcgtgtgtggtagtcatgtt | SEQ ID NO:278 |
| Jo.a.pos.4.cr | cgtgtgtggtagtcatgttctg | SEQ ID NO:279 |
| Jo.a.pos.5.cr | gtggtagtcatgttctgggg | SEQ ID NO:280 |
| Dombrock-Joseph(a) negative allele: | | |
| Jo.a.neg.1 | ctaccccagaacatgactatcac | SEQ ID NO:281 |
| Jo.a.neg.2 | cccagaacatgactatcacaca | SEQ ID NO:282 |
| Jo.a.neg.3 | cagaacatgactatcacacacgc | SEQ ID NO:283 |
| Jo.a.neg.4 | catgactatcacacacgctgtg | SEQ ID NO:284 |
| Jo.a.neg.5 | actatcacacacgctgtggc | SEQ ID NO:285 |
| Jo.a.neg.1.cr | aatagccacagcgtgtgtgatagt | SEQ ID NO:286 |
| Jo.a.neg.2.cr | cacagcgtgtgtgatagtcatg | SEQ ID NO:287 |
| Jo.a.neg.3.cr | cagcgtgtgtgatagtcatgtt | SEQ ID NO:288 |
| Jo.a.neg.4.cr | cgtgtgtgatagtcatgttctgg | SEQ ID NO:289 |
| Jo.a.neg.5.cr | gtgatagtcatgttctggggtag | SEQ ID NO:290 |
| Duffy-a-allele: | | |
| Fy.A.I | ccagatggagactatggtgcc | SEQ ID NO:291 |
| Fy.A.2 | atggagactatggtgccaac | SEQ ID NO:292 |
| Fy.A.3 | ggagactatggtgccaacctg | SEQ ID NO:293 |
| Fy.A.4 | gactatggtgccaacctgga | SEQ ID NO:294 |
| Fy.A.5 | tatggtgccaacctggaag | SEQ ID NO:295 |
| Fy.A.1.cr | cttccaggttggcaccata | SEQ ID NO:296 |
| Fy.A.2.cr | tccaggttggcaccatagtc | SEQ ID NO:297 |
| Fy.A.3.cr | aggttggcaccatagtctcc | SEQ ID NO:298 |
| Fy.A.4.cr | gttggcaccatagtctccat | SEQ ID NO:299 |
| Fy.A.5.cr | gcaccatagtctccatctgg | SEQ ID NO:300 |
| Duffy-b-allele: | | |
| Fy.B.1 | cccagatggagactatgatgcc | SEQ ID NO:301 |
| Fy.B.2 | gatggagactatgatgccaac | SEQ ID NO:302 |
| Fy.B.3 | tggagactatgatgccaacctg | SEQ ID NO:303 |
| Fy.B.4 | gactatgatgccaacctggaa | SEQ ID NO:304 |
| Fy.B.5 | tatgatgccaacctggaagc | SEQ ID NO:305 |
| Fy.B.1.cr | gcttccaggttggcatcata | SEQ ID NO:306 |
| Fy.B.2.cr | ttccaggttggcatcatagtc | SEQ ID NO:307 |
| Fy.B.3.cr | caggttggcatcatagtctcc | SEQ ID NO:308 |
| Fy.B.4.cr | gttggcatcatagtctccatc | SEQ ID NO:309 |
| Fy.B.5.cr | gcatcatagtctccatctggg | SEQ ID NO:310 |
| Duffy GATAbox-normal-allele: | | |
| Fy.GATA.normal.1 | agtccttggctcttatcttg | SEQ ID NO:311 |
| Fy.GATA.normal.2 | agtccttggctcttatcttgga | SEQ ID NO:312 |
| Fy.GATA.normal.3 | cttggctcttatcttggaagc | SEQ ID NO:313 |
| Fy.GATA.normal.4 | gctcttatcttggaagcacagg | SEQ ID NO:314 |
| Fy.GATA.normal.5 | cttatcttggaagcacaggcgc | SEQ ID NO:315 |
| Fy.GATA.normal.1.cr | gcctgtgcttccaagataag | SEQ ID NO:316 |
| Fy.GATA.normal.2.cr | tgtgcttccaagataagagc | SEQ ID NO:317 |
| Fy.GATA.normal.3.cr | tgcttccaagataagagcca | SEQ ID NO:318 |
| Fy.GATA.normal.4.cr | ttccaagataagagccaagga | SEQ ID NO:319 |
| Fy.GATA.normal.5.cr | caagataagagccaaggact | SEQ ID NO:320 |
| Duffy GATAbox-mutation-allele: | | |
| Fy.GATA.mut.1 | tccttggctcttaccttg | SEQ ID NO:321 |
| Fy.GATA.mut.2 | tccttggctcttaccttgga | SEQ ID NO:322 |
| Fy.GATA.mut.3 | tggctcttaccttggaagc | SEQ ID NO:323 |
| Fy.GATA.mut.4 | gctcttaccttggaagcacag | SEQ ID NO:324 |
| Fy.GATA.mut.5 | cttaccttggaagcacaggcg | SEQ ID NO:325 |
| Fy.GATA.mut.1.cr | cctgtgcttccaaggtaag | SEQ ID NO:326 |
| Fy.GATA.mut.2.cr | gtgcttccaaggtaagagc | SEQ ID NO:327 |
| Fy.GATA.mut.3.cr | gcttccaaggtaagagcca | SEQ ID NO:328 |
| Fy.GATA.mut.4.cr | ttccaaggtaagagccaagg | SEQ ID NO:329 |
| Fy.GATA.mut.5.cr | caaggtaagagccaagga | SEQ ID NO:330 |
| Duffy Fyx-normal-allele: | | |
| Fy.X.(b).1 | ttttcagacctctcttccgct | SEQ ID NO:331 |
| Fy.X.(b).2 | agacctctcttccgctggc | SEQ ID NO:332 |
| Fy.X.(b).3 | ctctcttccgctggcagc | SEQ ID NO:333 |
| Fy.X.(b).4 | ctcttccgctggcagctc | SEQ ID NO:334 |
| Fy.X.(b).5 | cttccgctggcagctctg | SEQ ID NO:335 |
| Fy.X.(b).1.cr | cagagctgccagcggaa | SEQ ID NO:336 |
| Fy.X.(b).2.cr | agctgccagcggaagag | SEQ ID NO:337 |
| Fy.X.(b).3.cr | ctgccagcggaagagagg | SEQ ID NO:338 |
| Fy.X.(b).4.cr | gccagcggaagagaggtc | SEQ ID NO:339 |
| Fy.X.(b).5.cr | cagcggaagagaggtctg | SEQ ID NO:340 |
| Duffy Fyx-mutation-allele: | | |
| Fy.X.1 | gcttttcagacctctcttctgct | SEQ ID NO:341 |
| Fy.X.2 | tcagacctctcttctgctggc | SEQ ID NO:342 |
| Fy.X.3 | acctctcttctgctggcagc | SEQ ID NO:343 |
| Fy.X.4 | ctcttctgctggcagctctg | SEQ ID NO:344 |
| Fy.X.5 | cttctgctggcagctctgc | SEQ ID NO:345 |
| Fy.X.1.cr | gcagagctgccagcagaa | SEQ ID NO:346 |
| Fy.X.2.cr | gagctgccagcagaagagag | SEQ ID NO:347 |
| Fy.X.3.cr | agctgccagcagaagagagg | SEQ ID NO:348 |
| Fy.X.4.cr | gccagcagaagagaggtctg | SEQ ID NO:349 |
| Fy.X.5.cr | cagcagaagagaggtctgaaa | SEQ ID NO:350 |
| Kidd-allele-a: | | |
| Jk.a.1 | cagccccatttgaggaca | SEQ ID NO:351 |
| Jk.a.2 | gccccatttgaggacatcta | SEQ ID NO:352 |
| Jk.a.3 | ccatttgaggacatctactttg | SEQ ID NO:353 |
| Jk.a.4 | atttgaggacatctactttgga | SEQ ID NO:354 |
| Jk.a.5 | gaggacatctactttggactct | SEQ ID NO:355 |
| Jk.a.1.cr | cagagtccaaagtagatgtcctc | SEQ ID NO:356 |
| Jk.a.2.cr | agtccaaagtagatgtcctcaaa | SEQ ID NO:357 |
| Jk.a.3.cr | aaagtagatgtcctcaaatggg | SEQ ID NO:358 |
| Jk.a.4.cr | tagatgtcctcaaatggggc | SEQ ID NO:359 |
| Jk.a.5.cr | atgtcctcaaatggggctg | SEQ ID NO:360 |
| Kidd-allele-b: | | |
| Jk.b.1 | tcagccccatttgagaaca | SEQ ID NO:361 |
| Jk.b.2 | gccccatttgagaacatcta | SEQ ID NO:362 |
| Jk.b.3 | cccatttgagaacatctactttg | SEQ ID NO:363 |
| Jk.b.4 | atttgagaacatctactttggac | SEQ ID NO:364 |
| Jk.b.5 | gagaacatctactttggactctg | SEQ ID NO:365 |
| Jk.b.1.cr | ccagagtccaaagtagatgttctc | SEQ ID NO:366 |
| Jk.b.2.cr | agagtccaaagtagatgttctcaaa | SEQ ID NO:367 |
| Jk.b.3.cr | ccaaagtagatgttctcaaatgg | SEQ ID NO:368 |
| Jk.b.4.cr | agtagatgttctcaaatggggc | SEQ ID NO:369 |
| Jk.b.5.cr | atgttctcaaatggggctga | SEQ ID NO:370 |
| Kell-K1-allele: | | |
| KEL.1.1 | tccttaaactttaaccgaatgct | SEQ ID NO:371 |
| KEL.1.2 | ttaaactttaaccgaatgctgaga | SEQ ID NO:372 |
| KEL.1.3 | aactttaaccgaatgctgagactt | SEQ BD NO:373 |
| KEL.1.4 | aaccgaatgctgagacttctg | SEQ ID NO:374 |
| KEL.1.5 | cgaatgctgagacttctgatgag | SEQ ID NO:375 |
| KEL 1.6 CR | actcatcagaagtctcagcattc | SEQ ID NO:376 |
| KEL 1.7 CR | tcagaagtctcagcattcggt | SEQ ID NO:377 |
| KEL 1.8 CR | aagtctcagcattcggttaaag | SEQ ID NO:378 |
| KEL 1.9 CR | tctcagcattcggttaaagtttaa | SEQ ID NO:379 |
| KEL 1.10 CR | agcattcggttaaagtttaagga | SEQ ID NO:380 |
| Kell-K2-allele: | | |
| KEL.2.1 | ccttaaactttaaccgaacgct | SEQ ID NO:381 |
| KEL.2.2 | aactttaaccgaacgctgaga | SEQ ID NO:382 |
| KEL.2.3 | ctttaaccgaacgctgagactt | SEQ ID NO:383 |
| KEL.2.4 | aaccgaacgctgagacttct | SEQ ID NO:384 |
| KEL.2.5 | cgaacgctgagacttctgatg | SEQ ID NO:385 |
| KEL 2.6 CR | tcatcagaagtctcagcgttc | SEQ ID NO:386 |
| KEL 2.7 CR | cagaagtctcagcgttcggt | SEQ ID NO:387 |
| KEL 2.8 CR | agtctcagcgttcggttaaag | SEQ ID NO:388 |
| KEL 2.9 CR | tctcagcgttcggnaaagtt | SEQ ID NO:389 |
| KEL 2.10 CR | agcgttcggttaaagtttaagg | SEQ ID NO:390 |
| Kell-K3-allele: | | |
| KEL.3.1 | aatctccatcacttcatggct | SEQ ID NO:391 |
| KEL.3.2 | tccatcacttcatggctgtt | SEQ ID NO:392 |
| KEL.3.3 | atcacttcatggctgttcca | SEQ ID NO:393 |
| KEL.3.4 | acttcatggctgttccagtt | SEQ ID NO:394 |
| KEL.3.5 | tcatggctgttccagtttc | SEQ ID NO:395 |
| KEL.3.1.cr | agaaactggaacagccatgaa | SEQ ID NO:396 |
| KEL.3.2.cr | aactggaacagccatgaagtg | SEQ ID NO:397 |
| KEL.3.3.cr | tggaacagccatgaagtgatg | SEQ ID NO:398 |
| KEL.3.4.cr | acagccatgaagtgatggag | SEQ ID NO:399 |
| KEL.3.5.cr | gccatgaagtgatggagatt | SEQ ID NO:400 |
| Kell-K4-allele: | | |
| KELA1 | tctccatcacttcacggct | SEQ ID NO:401 |
| KEL.4.2 | ccatcacttcacggctgtt | SEQ ID NO:402 |
| KEL.4.3 | cacttcacggctgttcca | SEQ ID NO:403 |
| KEL.4.4 | acttcacggctgttccag | SEQ ID NO:404 |
| KEL.4.5 | tcacggctgttccagttt | SEQ ID NO:405 |
| KEL.4.1.cr | aaactggaacagccgtgaa | SEQ ID NO:406 |
| KEL.4.2.cr | ctggaacagccgtgaagtg | SEQ ID NO:407 |
| KEL.4.3.cr | ggaacagccgtgaagtgatg | SEQ ID NO:408 |
| KEL.4.4.cr | acagccgtgaagtgatgg | SEQ ID NO:409 |
| KEL.4.5.cr | gccgtgaagtgatggaga | SEQ ID NO:410 |
| Kell-K6-allele: | | |
| KEL.6.1 | tactgcctgggggctgccccgcc | SEQ ID NO:411 |
| KEL.6.2 | tgggggctgccccgcctgt | SEQ ID NO:412 |
| KEL.6.3 | gctgccccgcctgtgac | SEQ ID NO:413 |
| KEL.6.4 | ctgccccgcctgtgacaa | SEQ ID NO:414 |
| KEL.6.5 | gccccgcctgtgacaac | SEQ ID NO:415 |
| KEL.6.1.cr | gttgtcacaggcggggc | SEQ ID NO:416 |
| KEL.6.2.cr | ttgtcacaggcggggcag | SEQ ID NO:417 |
| KEL.6.3.cr | tcacaggcggggcagccc | SEQ ID NO:418 |
| KEL.6.4.cr | acaggcggggcagccccca | SEQ ID NO:419 |
| KEL.6.5.cr | aggcggggcagccccca | SEQ ID NO:420 |
| Kell-K7-allele: | | |
| KEL.7.1 | actgcctgggggctgcctcgcc | SEQ ID NO:421 |
| KEL.7.2 | cctgggggctgcctcgcctgt | SEQ ID NO:422 |
| KEL.7.3 | ggctgcctcgcctgtgac | SEQ ID NO:423 |
| KEL.7.4 | ctgcctcgcctgtgacaacc | SEQ ID NO:424 |
| KEL.7.5 | gcctcgcctgtgacaacc | SEQ ID NO:425 |
| KEL.7.1.cr | ggttgtcacaggcgaggc | SEQ ID NO:426 |
| KEL.7.2.cr | ggttgtcacaggcgaggcag | SEQ ID NO:427 |
| KEL.7.3.cr | ttgtcacaggcgaggcagccc | SEQ ID NO:428 |
| KEL.7.4.cr | acaggcgaggcagcccccagg | SEQ ID NO:429 |
| KEL.7.5.cr | aggcgaggcagcccccagg | SEQ ID NO:430 |
| Lutheran a-allele: | | |
| Lu.a.1 | ggagctcgcccccgcct | SEQ ID NO:431 |
| Lu.a.2 | gctcgcccccgcctagc | SEQ ID NO:432 |
| Lu.a.3 | cgcccccgcctagcctc | SEQ ID NO:433 |
| Lu.a.4 | cccccgcctagcctcgg | SEQ ID NO:434 |
| Lu.a.5 | cccgcctagcctcggct | SEQ ID NO.435 |
| Lu.a.1 CR | agccgaggctaggcggg | SEQ ID NO:436 |
| Lu.a.2 CR | ccgaggctaggcggggg | SEQ ID NO:437 |
| Lu.a.3 CR | gaggctaggcgggggcg | SEQ ID NO:438 |
| Lu.a.4 CR | gctaggcgggggcgagc | SEQ ID NO:439 |
| Lu.a.5 CR | aggcgggggcgagctcc | SEQ ID NO:440 |
| Lutheran b-allele: | | |
| Lu.b.1 | gggagctcgcccccacct | SEQ ID NO:441 |
| Lu.b.2 | gagctcgcccccacctagc | SEQ ID NO:442 |
| Lu.b.3 | ctcgcccccacctagcctc | SEQ ID NO:443 |
| Lu.b.4 | cccccacctagcctcggct | SEQ ID NO:444 |
| Lu.b.5 | cccacctagcctcggctga | SEQ ID NO:445 |
| Lu.b.1 CR | tcagccgaggctaggtggg | SEQ ID NO:446 |
| Lu.b.2 CR | agccgaggctaggtggggg | SEQ ID NO:447 |
| Lu.b.3 CR | gaggctaggtgggggcgag | SEQ ID NO:448 |
| Lu.b.4 CR | gctaggtgggggcgagctc | SEQ ID NO:449 |
| Lu.b.5 CR | aggtgggggcgagctccc | SEQ ID NO:450 |
| MNS M-allele: | | |
| M.1 | gtgagcatatcagcatcaag | SEQ ID NO:451 |
| M.2 | atcagcatcaagtaccactgg | SEQ ID NO:452 |
| M.3 | catcaagtaccactggtgtg | SEQ ID NO:453 |
| M.4 | taccactggtgtggcaatgc | SEQ ID NO:454 |
| M.5 | ctggtgtggcaatgcaca | SEQ ID NO:455 |
| M.1.cr | tgtgcattgccacaccagt | SEQ ID NO:456 |
| M.2.cr | gcattgccacaccagtggta | SEQ ID NO:457 |
| M.3.cr | ccacaccagtggtacttgatg | SEQ ID NO:458 |
| M.4.cr | accagtggtacttgatgct | SEQ ID NO:459 |
| M.5.cr | cttgatgctgatatgctcac | SEQ ID NO:460 |
| MNS N-allele: | | |
| N.1 | tgtgagcatatcagcattaag | SEQ ID NO:461 |
| N.2 | atcagcattaagtaccactgagg | SEQ ID NO:462 |
| N.3 | cattaagtaccactgaggtgg | SEQ ID NO:463 |
| N.4 | accactgaggtggcaatgc | SEQ ID NO:464 |
| N.5 | ctgaggtggcaatgcacact | SEQ ID NO:465 |
| N.1.cr | gtgtgcattgccacctcagt | SEQ ID NO:466 |
| N.2.cr | gcattgccacctcagtggta | SEQ ID NO:467 |
| N.3.cr | ccacctcagtggtacttaatgc | SEQ ID NO:468 |
| N.4.cr | cctcagtggtacttaatgct | SEQ ID NO:469 |
| N.5.cr | cttaatgctgatatgctcaca | SEQ ID NO:470 |
| MNS S-allele: | | |
| big.S.1 | tttgctttataggagaaatggga | SEQ ID NO:471 |
| big.S.2 | ctttataggagaaatgggaca | SEQ ID NO:472 |
| big.S.3 | ttataggagaaatgggacaacttg | SEQ ID NO:473 |
| big.S.4 | gagaaatgggacaacttgtcc | SEQ ID NO:474 |
| big.S.5 | aaatgggacaacttgtccatc | SEQ ID NO:475 |
| big.S.1.cr | gatggacaagttgtcccattt | SEQ ID NO:476 |
| big.S.2.cr | gacaagttgtcccatttctcc | SEQ ID NO:477 |
| big.S.3.cr | aagttgtcccatttctcctata | SEQ ID NO:478 |
| big.S.4.cr | tgtcccatttctcctataaagca | SEQ ID NO:479 |
| big.S.5.cr | cccatttctcctataaagcaaaa | SEQ ID NO:480 |
| MNS s-allele: | | |
| little.s.1 | tgctttataggagaaacggga | SEQ ID NO:481 |
| little.s.2 | tttataggagaaacgggaca | SEQ ID NO:482 |
| little.s.3 | ggagaaacgggacaacttg | SEQ ID NO:483 |
| little.s.4 | gagaaacgggacaacttgtc | SEQ ID NO:484 |
| little.s.5 | aaacgggacaacttgtccat | SEQ ID NO:485 |
| little.s.1.cr | tggacaagttgtcccgttt | SEQ ID NO:486 |
| little.s.2.cr | acaagttgtcccgtttctcc | SEQ ID NO:487 |
| little.s.3.cr | agttgtcccgtttctcctata | SEQ ID NO:488 |
| little.s.4.cr | tgtcccgtttctcctataaagc | SEQ ID NO:489 |
| little.s.5.cr | cccgttctcctataaagca | SEQ ID NO:490 |
| MNS U-positive-allele: | | |
| U.pos.1 | ttgctgctctctttagctcc | SEQ ID NO:491 |
| U.pos.2 | ctctctttagctcctgtagtgat | SEQ ID NO:492 |
| U.pos.3 | agctcctgtagtgataatactca | SEQ ID NO:493 |
| U.pos.4 | gtagtgataatactcattatttttg | SEQ ID NO:494 |
| U.pos.5 | taatactcattatttttggggtg | SEQ ID NO:495 |
| U.pos.1.cr | caccccaaaaataatgagtatta | SEQ ID NO:496 |
| U.pos.2.cr | caaaaataatgagtattatcactaca | SEQ ID NO:497 |
| U.pos.3.cr | agtattatcactacaggagctaaa | SEQ ID NO:498 |
| U.pos.4.cr | atcactacaggagctaaagag | SEQ ID NO:499 |
| U.pos.5.cr | gagctaaagagagcagcaaa | SEQ ID NO:500 |
| MNS U-negative-allele: | | |
| U.neg.1 | ttttgctgctctctttatctcc | SEQ ID NO:501 |
| U.neg.2 | gctctctttatctcctgtagagat | SEQ ID NO:502 |
| U.neg.3 | tatctcctgtagagataacactca | SEQ ID NO:503 |
| U.neg.4 | gtagagataacactcattattttt | SEQ ID NO:504 |
| U.neg.5 | taacactcattatttttggggt | SEQ ID NO:505 |
| U.neg.1.cr | accccaaaaataatgagtgtta | SEQ ID NO:506 |
| U.neg.2.cr | aaaaataatgagtgttatctctaca | SEQ ID NO:507 |
| U.neg.3.cr | agtgttatctctacaggagataaa | SEQ ID NO:508 |
| U.neg.4.cr | atctctacaggagataaagagag | SEQ ID NO:509 |
| U.neg.5.cr | gagataaagagagcagcaaaatta | SEQ ID NO:510 |
| Rhesus C-allele(307T): | | |
| Rh.big.C.1 | tgagccagttcccttctgg | SEQ ID NO:511 |
| Rh.big.C.2 | gagccagttcccttctgg | SEQ ID NO:512 |
| Rh.big.C.3 | ctgagccagttcccttctg | SEQ ID NO:513 |
| Rh.big.C.4 | ccttctgggaaggtggtc | SEQ ID NO:514 |
| Rh.big.C.5 | ccttctgggaaggtggtca | SEQ ID NO:515 |
| Rh.big.C.1.cr | tgaccaccttcccagaagg | SEQ ID NO:516 |
| Rh.big.C.2.cr | gaccaccttcccagaagg | SEQ ID NO:517 |
| Rh.big.C.3.cr | ccagaagggaactggctc | SEQ ID NO:518 |
| Rh.big.C.4.cr | ccagaagggaactggctca | SEQ ID NO:519 |
| Rh.big.C.5.cr | cagaagggaactggctcag | SEQ ID NO:520 |
| Rhesus c-allele(307C): | | |
| Rh.little.c.1 | gagccagttccctcctgg | SEQ ID NO:521 |
| Rh.little.c.2 | agccagttccctcctgg | SEQ ID NO:522 |
| Rh.little.c.3 | tgagccagttccctcctg | SEQ ID NO:523 |
| Rh.little.c.4 | cctcctgggaaggtggt | SEQ ID NO:524 |
| Rh.little.c.5 | cctcctgggaaggtggtc | SEQ ID NO:525 |
| Rh.little.c.1.cr | gaccaccttcccaggagg | SEQ ID NO:526 |
| Rh.little.c.2.cr | accaccttcccaggagg | SEQ ID NO:527 |
| Rh.little.c.3.cr | ccaggagggaactggct | SEQ ID NO:528 |
| Rh.little.c.4.cr | ccaggagggaactggctc | SEQ ID NO:529 |
| Rh.little.c.5.cr | caggagggaactggctca | SEQ ID NO:530 |
| Rhesus BigC-intron2-specific-insert-allele: | | |
| RhC.intron.2.1 | agggtgccctttgtcacttc | SEQ ID NO:531 |
| RhC.intron.2.2 | gccctttgtcacttcccagt | SEQ ID NO:532 |
| RhC.intron.2.3 | cctttgtcacttcccagtgg | SEQ ID NO:533 |
| RhC.intron.2.4 | ttgtcacttcccagtggtacaa | SEQ ID NO:534 |
| RhC.intron.2.5 | tcacttcccagtggtacaatca | SEQ ID NO:535 |
| RhC.intron.2.1.cr | gaagtgacaaagggcaccct | SEQ ID NO:536 |
| RhC.intron.2.2.cr | actgggaagtgacaaagggc | SEQ ID NO:537 |
| RhC.intron.2.3.cr | ccactgggaagtgacaaagg | SEQ ID NO:538 |
| RhC.intron.2.4.cr | tgtaccactgggaagtgacaaa | SEQ ID NO:539 |
| RhC.intron.2.5.cr | tgattgtaccactgggaagtga | SEQ ID NO:540 |
| Rhesus E-allele: | | |
| Rh.big.E.1 | gccaagtgtcaactctcctct | SEQ ID NO:541 |
| Rh.big.E.2 | caagtgtcaactctcctctgct | SEQ ID NO:542 |
| Rh.big.E.3 | gtgtcaactctcctctgctgag | SEQ ID NO:543 |
| Rh.big.E.4 | caactctcctctgctgagaagtc | SEQ ID NO:544 |
| Rh.big.E.5 | tctcctctgctgagaagtcc | SEQ ID NO:545 |
| Rh.big.E.1.cr | ggacttctcagcagaggagag | SEQ ID NO:546 |
| Rh.big.E.2.cr | cttctcagcagaggagagttga | SEQ ID NO:547 |
| Rh.big.E.3.cr | ctcagcagaggagagttgacac | SEQ ID NO:548 |
| Rh.big.E.4.cr | gcagaggagagttgacacttg | SEQ ID NO:549 |
| Rh.big.E.5.cr | gaggagagttgacacttggc | SEQ ID NO:550 |
| Rhesus e-allele: | | |
| Rh.little.e.1 | gccaagtgtcaactctgctct | SEQ ID NO:551 |
| Rh.little.e.2 | caagtgtcaactctgctctgct | SEQ ID NO:552 |
| Rh.little.e.3 | tgtcaactctgctctgctgag | SEQ ID NO:553 |
| Rh.little.e.4 | caactctgctctgctgagaag | SEQ ID NO:554 |
| Rh.little.e.5 | tctgctctgctgagaagtcc | SEQ ID NO:555 |
| Rh.little.e.1.cr | ggacttctcagcagagcagag | SEQ ID NO:556 |
| Rh.little.e.2.cr | ttctcagcagagcagagttga | SEQ ID NO:557 |
| Rh.little.e.3.cr | tcagcagagcagagttgacac | SEQ ID NO:558 |
| Rh.little.e.4.cr | gcagagcagagttgacacttg | SEQ ID NO:559 |
| Rh.little.e.5.cr | gagcagagttgacacttggc | SEQ ID NO:560 |
| Rhesus RHD-allele: | | |
| RhD 1 | ttccccacagctccatcat | SEQ ID NO:561 |
| RhD 2 | acagctccatcatgggctac | SEQ ID NO:562 |
| RhD 3 | tccatcatgggctacaacttc | SEQ ID NO:563 |
| RhD 4 | tcatgggctacaacttcagct | SEQ ID NO:564 |
| RhD 5 | gggctacaacttcagcttgct | SEQ ID NO:565 |
| RhD cr 1 | agcaagctgaagttgtagccc | SEQ ID NO:566 |
| RhD cr 2 | agctgaagttgtagcccatga | SEQ ID NO:567 |
| RhD cr 3 | gaagttgtagcccatgatgg | SEQ ID NO:568 |
| RhD cr 4 | gcccatgatggagctgt | SEQ ID NO:569 |
| RhD cr 5 | tgatggagctgtggggaa | SEQ ID NO:570 |
| Rhesus r's-allele: | | |
| r's.T.1 | ggaaggtcaacttggtgca | SEQ ID NO:571 |
| r's.T.2 | ggaaggtcaacttggtgcagt | SEQ ID NO:572 |
| r's.T.3 | caacttggtgcagttggtg | SEQ ID NO:573 |
| r's.T.4 | acttggtgcagttggtggt | SEQ ID NO:574 |
| r's.T.5 | ttggtgcagttggtggtgat | SEQ ID NO:575 |
| r's.T.1.cr | catcaccaccaactgcacca | SEQ ID NO:576 |
| r's.T.2.cr | caccaccaactgcaccaagt | SEQ ID NO:577 |
| r's.T.3.cr | accaactgcaccaagttgac | SEQ ID NO:578 |
| r's.T.4.cr | actgcaccaagttgaccttcc | SEQ ID NO:579 |
| r's.T.5.cr | tgcaccaagttgaccttcc | SEQ ID NO:580 |
| Rhesus DVI-allele: | | |
| Rh.DVI.1 | atttcaaccctcttggcctt | SEQ ID NO:581 |
| Rh.DVI.2 | aaccctcttggcctttgttt | SEQ ID NO:582 |
| Rh.DVI.3 | tcttggcctttgtttccttg | SEQ ID NO:583 |
| Rh.DVI.4 | ggtatcagcttgagagctcg | SEQ ID NO:584 |
| Rh.DVI.5 | atcagcttgagagctcggag | SEQ ID NO:585 |
| Rh.DVI.1.cr | aaggccaagagggttgaaat | SEQ ID NO:586 |
| Rh.DVI.2.cr | aaacaaaggccaagagggtt | SEQ ID NO:587 |
| Rh.DVI.3.cr | caaggaaacaaaggccaaga | SEQ ID NO:588 |
| Rh.DVI.4.cr | cgagctctcaagctgatacc | SEQ ID NO:589 |
| Rh.DVI.5.cr | ctccgagctctcaagctgat | SEQ ID NO:590 |
| Rhesus RHD-Pseudogene-mutation-allele: | | |
| RhD Y 1 | tttctttgcagacttaggtgc | SEQ ID NO:591 |
| RhD Y 2 | ctttgcagacttaggtgcaca | SEQ ID NO:592 |
| RhD Y 3 | tttgcagacttaggtgcacagt | SEQ ID NO:593 |
| RhD Y 4 | acttaggtgcacagtgcgg | SEQ ID NO:594 |
| RhD Y 5 | cttaggtgcacagtgcggt | SEQ ID NO:595 |
| RhD Y cr 1 | caccgcactgtgcacctaa | SEQ ID NO:596 |
| RhD Y cr 2 | ccgcactgtgcacctaagtc | SEQ ID NO:597 |
| RhD Y cr 3 | gcactgtgcacctaagtctgc | SEQ ID NO:598 |
| RhD Y cr 4 | tgcacctaagtctgcaaaga | SEQ ID NO:599 |
| RhD Y cr 5 | cacctaagtctgcaaagaaatagcg | SEQ ID NO:600 |
| Rhesus RHD-Pseudogene-normal-allele: | | |
| RhD non Y 1 | ctatttctttgcagacttatgtgc | SEQ ID NO:601 |
| RhD non Y 2 | tctttgcagacttatgtgcaca | SEQ ID NO:602 |
| RhD non Y 3 | ttgcagacttatgtgcacagtg | SEQ ID NO:603 |
| RhD non Y 4 | acttatgtgcacagtgcggt | SEQ ID NO:604 |
| RhD non Y 5 | cttatgtgcacagtgcggtg | SEQ ID NO:605 |
| RhD non Y cr 1 | acaccgcactgtgcacataa | SEQ ID NO:606 |
| RhD non Y cr 2 | accgcactgtgcacataagtc | SEQ ID NO:607 |
| RhD non Y cr 3 | gcactgtgcacataagtctgc | SEQ ID NO:608 |
| RhD non Y cr 4 | tgtgcacataagtctgcaaag | SEQ ID NO:609 |
| RhD non Y cr 5 | cacataagtctgcaaagaaatagcg | SEQ ID NO:610 |
| Yt-a-allele: | | |
| Yt.a.1 | gcgggagacttccacgg | SEQ ID NO:611 |
| Yt.a.2 | gggagacttccacggcct | SEQ ID NO:612 |
| Yt.a.3 | gagacttccacggcctgc | SEQ ID NO:613 |
| Yt.a.4 | gacttccacggcctgca | SEQ ID NO:614 |
| Yt.a.5 | ttccacggcctgcaggta | SEQ ID NO:615 |
| Yt.a.1 CR | tacctgcaggccgtggaa | SEQ ID NO:616 |
| Yt.a.2 CR | tgcaggccgtggaagtc | SEQ ID NO:617 |
| Yt.a.3 CR | gcaggccgtggaagtctc | SEQ ID NO:618 |
| Yt.a.4 CR | aggccgtggaagtctccc | SEQ ID NO:619 |
| Yt.a.5 CR | ccgtggaagtctcccgc | SEQ ID NO:620 |
| Yt-b-allele: | | |
| Yt.b.1 | gcgggagacttcaacggc | SEQ ID NO:621 |
| Yt.b.2 | gggagacttcaacggcctg | SEQ ID NO:622 |
| Yt.b.3 | gagacttcaacggcctgca | SEQ ID NO:623 |
| Yt.b.4 | gacttcaacggcctgcag | SEQ ID NO:624 |
| Yt.b.5 | ttcaacggcctgcaggtaa | SEQ ID NO:625 |
| Yt.b.1 CR | ttacctgcaggccgttgaa | SEQ ID NO:626 |
| Yt.b.2 CR | ctgcaggccgttgaagtc | SEQ ID NO:627 |
| Yt.b.3 CR | tgcaggccgttgaagtctc | SEQ ID NO:628 |
| Yt.b.4 CR | caggccgttgaagtctccc | SEQ ID NO:629 |
| Yt.b.5 CR | gccgttgaagtctcccgc | SEQ ID NO:630 |

14. The method of claim 10, wherein the oligonucleotide probes comprise at their 5′ end a linker and a reactive group for attachment to an array support.

15. The method of claim 10, wherein the probes include one or more oligonucleotides with a sequence not occurring in the DNA of said mammalian species to allow for background subtraction.

16. The method of claim 10, wherein the probes include one or more positive hybridization controls,
and its detectably labeled complement is added to the products of the multiplex PCR amplification.

17. The method of claim 10, wherein the probes, before adding the products of the multiplex PCR amplification, are subjected to prehybridization and DNA denaturation treatments.

18. The method of claim 10, wherein the denatured products of the multiplex PCR amplification are applied to the probes without prepurification.

19. The method of claim 10, wherein for each blood cell antigen the ratio of the signal intensities for each of the alleles is used to assign the genotype.

20. A kit for genotyping blood cell antigens by the method of claim 1, comprising one pair of blood cell antigen-specific chimeric primers for each blood cell antigen to be genotyped and at least one detectably labeled universal primer.

21. The kit of claim 20, further comprising a DNA array of probes.

22. A set of blood cell antigen-specific chimeric primer pairs useful in a multiplex PCR, comprising at least two primer pairs selected from the group as defined in claim 7.

23. A set of blood cell antigen allele-specific oligonucleotide probes useful for genotyping blood cell antigens, comprising at least 72 different probes selected from the group as defined in claim 13.

24. The method of claim 10, wherein said probes are contained in a supported array.

25. The method of claim 15, wherein said probes are selected from the group consisting of

| | | |
|---|---|---|
| a3 | cagaccataagcacaggcgt, | SEQ ID NO:631 |
| a9 | gctcgtccacagtgcgttat, | SEQ ID NO:632 |
| a17 | cggcgttcaagcaaaccgaa, | SEQ ID NO:633 |
| a23 | gacatatagctccactcaga, | SEQ ID NO:634 |
| a27 | tagggtactgatgagcactc, | SEQ ID NO:635 |
| a33 | tcagccctatcgcaggatgt, | SEQ ID NO:169 |
| a35 | gagacacttgacagtagcca, | SEQ ID NO:170 |
| a38 | ggcagggcacctcagtttat, | SEQ ID NO:171 |
| a42 | tcaccagccagactgtgtag, | SEQ ID NO:172 |
| a43 | cttcacgcaagttgtccaga. | SEQ ID NO:173 |

26. The method of claim 16, wherein said positive hybridization control is

| | | |
|---|---|---|
| CS05 | gtcctgacttctagctcatg. | SEQ ID NO:174 |


SEQUENCE LISTING (651 sequences)

SEQ ID NO: 1 - DNA, 18 bp, Artificial Sequence
  forward primer MAPH
ggccgcgggaattcgatt

SEQ ID NO: 2 - DNA, 18 bp, Artificial Sequence
  reverse primer MAPH
gccgcgaattcactagtg

SEQ ID NO: 3 - DNA, 37 bp, Artificial Sequence
  primer HPA1-left
gccgcgaattcactagtgcttcaggtcacagcgaggt

SEQ ID NO: 4 - DNA, 38 bp, Artificial Sequence
  primer HPA1-right
ggccgcgggaattcgattgctccaatgtacggggtaaa

SEQ ID NO: 5 - DNA, 38 bp, Artificial Sequence
  primer HPA2-left
gccgcgaattcactagtgtgaaaggcaatgagctgaag

SEQ ID NO: 6 - DNA, 38 bp, Artificial Sequence
  primer HPA2-right
ggccgcgggaattcgattagccagactgagcttctcca

SEQ ID NO: 7 - DNA, 36 bp, Artificial Sequence
  primer HPA3-left
gccgcgaattcactagtggcctgaccactcctttgc

SEQ ID NO: 8 - DNA, 38 bp, Artificial Sequence
  primer HPA3-right
ggccgcgggaattcgattggaagatctgtctgcgatcc

SEQ ID NO: 9 - DNA, 38 bp, Artificial Sequence
  primer HPA4-left
gccgcgaattcactagtgatccgcaggttactggtgag

SEQ ID NO: 10 - DNA, 38 bp, Artificial Sequence
  HPA4-right
ggccgcgggaattcgattccatgaaggatgatctgtgg

SEQ ID NO: 11 - DNA, 42 bp, Artificial Sequence
  primer HPA5-left
gccgcgaattcactagtgtccaaatgcaagttaaattaccag

SEQ ID NO: 12 - DNA, 39 bp, Artificial Sequence
  primer HPA5-right
ggccgcgggaattcgattacagacgtgctcttggtaggt

SEQ ID NO: 13 - DNA, 43 bp, Artificial Sequence
  primer HPA15-left
gccgcgaattcactagtgtgtatcagttcttggttttgtgatg

SEQ ID NO: 14 - DNA, 38 bp, Artificial Sequence
  primer HPA15-right
ggccgcgggaattcgattaaaaccagtagccacccaag

SEQ ID NO: 15 - DNA, 38 bp, Artificial Sequence
  primer JK1/2-left
gccgcgaattcactagtggtctttcagccccatttgag

SEQ ID NO: 16 - DNA, 38 bp, Artificial Sequence
  primer JK1/2-right
ggccgcgggaattcgattgttgaaaccccagagtccaa

SEQ ID NO: 17 - DNA, 42 bp, Artificial Sequence
  primer FY1/2-left
gccgcgaattcactagtggaattcttcctatggtgtgaatga

SEQ ID NO: 18 - DNA, 38 bp, Artificial Sequence
  primer FY1/2-right
ggccgcgggaattcgattaagaagggcagtgcagagtc

SEQ ID NO: 19 - DNA, 37 bp, Artificial Sequence
  primer GATAbox-left
gccgcgaattcactagtgggccctcattagtccttgg

SEQ ID NO: 20 - DNA, 39 bp, Artificial Sequence
  primer GATAbox-right
ggccgcgggaattcgattgaaatgaggggcatagggata

SEQ ID NO: 21 - DNA, 38 bp, Artificial Sequence
  primer KEL1/2-left
gccgcgaattcactagtgaagggaaatggccatactga

SEQ ID NO: 22 - DNA, 38 bp, Artificial Sequence
  primer KEL1/2-right
ggccgcgggaattcgattagctgtgtaagagccgatcc

SEQ ID NO: 23 - DNA, 38 bp, Artificial Sequence
  primer KEL3/4-left
gccgcgaattcactagtggcctcagaaactggaacagc

SEQ ID NO: 24 - DNA, 39 bp, Artificial Sequence
  primer KEL3/4-right
ggccgcgggaattcgattagcaaggtgcaagaacactct

SEQ ID NO: 25 - DNA, 35 bp, Artificial Sequence
  primer RHCEex2for
gccgcgaattcactagtgcgtctgcttccccctcc

SEQ ID NO: 26 - DNA, 39 bp, Artificial Sequence
  primer RHex2rev
ggccgcgggaattcgattctgaacagtgtgatgaccacc

SEQ ID NO: 27 - DNA, 38 bp, Artificial Sequence
  primer RHCEex5for
gccgcgaattcactagtggccctcttcttgtggatgtt

SEQ ID NO: 28 - DNA, 38 bp, Artificial Sequence
  primer RHex5rev
ggccgcgggaattcgattggctgtcaccacactgactg

SEQ ID NO: 29 - DNA, 37 bp, Artificial Sequence
  primer RHD psi-left
ggccgcgggaattcgattgtagtgagctggcccatca

SEQ ID NO: 30 - DNA, 41 bp, Artificial Sequence
  primer RHD psi-right
gccgcgaattcactagtgtgtctagtttcttaccggcaagt

SEQ ID NO: 31 - DNA, 42 bp, Artificial Sequence
  primer RHD-leftB
gccgcgaattcactagtgttataataacacttgtccacaggg

SEQ ID NO: 32 - DNA, 34 bp, Artificial Sequence
  primer RHD-rightC
ggccgcgggaattcgattcggctccgacggtatc

SEQ ID NO: 33 - DNA, 35 bp, Artificial Sequence
  primer BigC-left
gccgcgaattcactagtgggccaccaccatttgaa

SEQ ID NO: 34 - DNA, 38 bp, Artificial Sequence
  primer BigC-rightintron 2
ggccgcgggaattcgattccatgaacatgccacttcac

SEQ ID NO: 35 - DNA, 39 bp, Artificial Sequence
  primer MN-left
gccgcgaattcactagtgtgagggaatttgtcttttgca

SEQ ID NO: 36 - DNA, 38 bp, Artificial Sequence
  primer MN-right
ggccgcgggaattcgattcagaggcaagaattcctcca

SEQ ID NO: 37 - DNA, 44 bp, Artificial Sequence
  primer U-left
gccgcgaattcactagtgcgctgatgttatctgtcttatttttc

SEQ ID NO: 38 - DNA, 40 bp, Artificial Sequence
  primer U-right
ggccgcgggaattcgattgatcgttccaataataccagcc

SEQ ID NO: 39 - DNA, 38 bp, Artificial Sequence
  primer JO-left
gccgcgaattcactagtgcctggcttaaccaaggaaaa

SEQ ID NO: 40 - DNA, 39 bp, Artificial Sequence
  primer JO-right
ggccgcgggaattcgatttcatactgctgtggagtcctg

SEQ ID NO: 41 - DNA, 19 bp, Artificial Sequence
  probe HPA-1aa
tacaggccctgcctctggg

SEQ ID NO: 42 - DNA, 18 bp, Artificial Sequence
  probe HPA-1ab
aggccctgcctctgggct

SEQ ID NO: 43 - DNA, 19 bp, Artificial Sequence
  probe HPA-1ac
ccctgcctctgggctcacc

SEQ ID NO: 44 - DNA, 19 bp, Artificial Sequence
  probe HPA-1ad
tgcctctgggctcacctcg

SEQ ID NO: 45 - DNA, 19 bp, Artificial Sequence
  probe HPA-1ae
ctctgggctcacctcgctg

SEQ ID NO: 46 - DNA, 19 bp, Artificial Sequence
  probe HPA-1aa CR
cagcgaggtgagcccagag

SEQ ID NO: 47 - DNA, 19 bp, Artificial Sequence
  probe HPA-1ab CR
cgaggtgagcccagaggca

SEQ ID NO: 48 - DNA, 19 bp, Artificial Sequence
  probe HPA-1ac CR
ggtgagcccagaggcaggg

SEQ ID NO: 49 - DNA, 18 bp, Artificial Sequence
  probe HPA-1ad CR
agcccagaggcagggcct

SEQ ID NO: 50 - DNA, 19 bp, Artificial Sequence
  probe HPA-1ae CR
cccagaggcagggcctgta

SEQ ID NO: 51 - DNA, 19 bp, Artificial Sequence
  probe HPA-1ba
tacaggccctgcctccggg

SEQ ID NO: 52 - DNA, 17 bp, Artificial Sequence
  probe HPA-1bb
aggccctgcctccggct

SEQ ID NO: 53 - DNA, 18 bp, Artificial Sequence
  probe HPA-1bc
ccctgcctccgggctcac

SEQ ID NO: 54 - DNA, 18 bp, Artificial Sequence
  probe HPA-1bd
ctgcctccgggctcacct

SEQ ID NO: 55 - DNA, 18 bp, Artificial Sequence
  probe HPA-1be
ctccgggctcacctcgct

SEQ ID NO: 56 - DNA, 18 bp, Artificial Sequence
  probe HPA-1ba CR
agcgaggtgagcccggag

SEQ ID NO: 57 - DNA, 18 bp, Artificial Sequence
  probe HPA-1bb CR
aggtgagcccggaggcag

SEQ ID NO: 58 - DNA, 18 bp, Artificial Sequence
  probe HPA-1bc CR
gtgagcccggaggcaggg

SEQ ID NO: 59 - DNA, 18 bp, Artificial Sequence
  probe HPA-1bd CR
agcccggaggcagggcct

SEQ ID NO: 60 - DNA, 19 bp, Artificial Sequence
  probe HPA-1be CR
cccggaggcagggcctgta

SEQ ID NO: 61 - DNA, 18 bp, Artificial Sequence
  probe HPA-2aa
ctgacgcccacacccaag

SEQ ID NO: 62 - DNA, 18 bp, Artificial Sequence
  probe HPA-2ab
ctcctgacgcccacaccc

SEQ ID NO: 63 - DNA, 18 bp, Artificial Sequence
  probe HPA-2ac
ggctcctgacgcccacac

SEQ ID NO: 64 - DNA, 18 bp, Artificial Sequence
  probe HPA-2ad
agggctcctgacgcccac

SEQ ID NO: 65 - DNA, 18 bp, Artificial Sequence
  probe HPA-2ae
ccagggctcctgacgccc

SEQ ID NO: 66 - DNA, 18 bp, Artificial Sequence
  probe HPA-2aa CR
cttgggtgtgggcgtcag

SEQ ID NO: 67 - DNA, 18 bp, Artificial Sequence
  probe HPA-2ab CR
gggtgtgggcgtcaggag

SEQ ID NO: 68 - DNA, 18 bp, Artificial Sequence
  probe HPA-2ac CR
gtgtgggcgtcaggagcc

SEQ ID NO: 69 - DNA, 18 bp, Artificial Sequence
  probe HPA-2ad CR
gtgggcgtcaggagccct

SEQ ID NO: 70 - DNA, 18 bp, Artificial Sequence
  probe HPA-2ae CR
gggcgtcaggagccctgg

SEQ ID NO: 71 - DNA, 19 bp, Artificial Sequence
  probe HPA-2ba
cctgatgcccacacccaag

SEQ ID NO: 72 - DNA, 19 bp, Artificial Sequence
  probe HPA-2bb
ctcctgatgcccacaccca

SEQ ID NO: 73 - DNA, 19 bp, Artificial Sequence
  probe HPA-2bc
ggctcctgatgcccacacc

SEQ ID NO: 74 - DNA, 19 bp, Artificial Sequence
  probe HPA-2bd
agggctcctgatgcccaca

SEQ ID NO: 75 - DNA, 18 bp, Artificial Sequence
  probe HPA-2be
ccagggctcctgatgccc

SEQ ID NO: 76 - DNA, 19 bp, Artificial Sequence
  probe HPA-2ba CR
cttgggtgtgggcatcagg

SEQ ID NO: 77 - DNA, 19 bp, Artificial Sequence
  probe HPA-2bb CR
tgggtgtgggcatcaggag

SEQ ID NO: 78 - DNA, 19 bp, Artificial Sequence
  probe HPA-2bc CR
ggtgtgggcatcaggagcc

SEQ ID NO: 79 - DNA, 19 bp, Artificial Sequence
  probe HPA-2bd CR
tgtgggcatcaggagccct

SEQ ID NO: 80 - DNA, 18 bp, Artificial Sequence
  probe HPA-2be CR
gggcatcaggagccctgg

SEQ ID NO: 81 - DNA, 18 bp, Artificial Sequence
  probe HPA-3aa
ccatccccagcccctccc

SEQ ID NO: 82 - DNA, 18 bp, Artificial Sequence
  probe HPA-3ab
gcccatccccagcccctc

SEQ ID NO: 83 - DNA, 18 bp, Artificial Sequence
  probe HPA-3ac
ctgcccatccccagcccc

SEQ ID NO: 84 - DNA, 18 bp, Artificial Sequence
  probe HPA-3ad1
gctgcccatccccagccc

SEQ ID NO: 85 - DNA, 18 bp, Artificial Sequence
  probe HPA-3ad
ggctgcccatccccagcc

SEQ ID NO: 86 - DNA, 18 bp, Artificial Sequence
  probe HPA-3ad2
gggctgcccatccccagc

SEQ ID NO: 87 - DNA, 17 bp, Artificial Sequence
  probe HPA-3ae
ggggctgcccatcccca

SEQ ID NO: 88 - DNA, 18 bp, Artificial Sequence
  probe HPA-3aa CR
gggaggggctggggatgg

SEQ ID NO: 89 - DNA, 18 bp, Artificial Sequence
  probe HPA-3ab CR
gaggggctggggatgggc

SEQ ID NO: 90 - DNA, 18 bp, Artificial Sequence
  probe HPA-3ac CR
ggggctggggatgggcag

SEQ ID NO: 91 - DNA, 18 bp, Artificial Sequence
  probe HPA-3ad1 CR
gggctggggatgggcagc

SEQ ID NO: 92 - DNA, 18 bp, Artificial Sequence
  probe HPA-3ad CR
ggctggggatgggcagcc

SEQ ID NO: 93 - DNA, 18 bp, Artificial Sequence
  probe HPA-3ad2 CR
gctggggatgggcagccc

SEQ ID NO: 94 - DNA, 17 bp, Artificial Sequence
  probe HPA-3ae CR
tggggatgggcagcccc

SEQ ID NO: 95 - DNA, 17 bp, Artificial Sequence
  probe HPA-3ba
ccagccccagcccctcc

SEQ ID NO: 96 - DNA, 17 bp, Artificial Sequence
  probe HPA-3bb
gcccagccccagcccct

SEQ ID NO: 97 - DNA, 17 bp, Artificial Sequence
  probe HPA-3bc
ctgcccagccccagccc

SEQ ID NO: 98 - DNA, 17 bp, Artificial Sequence
  probe HPA-3bd1
gctgcccagccccagcc

SEQ ID NO: 99 - DNA, 17 bp, Artificial Sequence
  probe HPA-3bd
ggctgcccagccccagc

SEQ ID NO: 100 - DNA, 17 bp, Artificial Sequence
  probe HPA-3bd2
gggctgcccagccccag

SEQ ID NO: 101 - DNA, 17 bp, Artificial Sequence
  probe HPA-3be
ggggctgcccagcccca

SEQ ID NO: 102 - DNA, 17 bp, Artificial Sequence
  probe HPA-3ba CR
ggaggggctggggctgg

SEQ ID NO: 103 - DNA, 17 bp, Artificial Sequence
  probe HPA-3bb CR
aggggctggggctgggc

SEQ ID NO: 104 - DNA, 17 bp, Artificial Sequence
  probe HPA-3bc CR
gggctggggctgggcag

SEQ ID NO: 105 - DNA, 17 bp, Artificial Sequence
  probe HPA-3bd1 CR
ggctggggctgggcagc

SEQ ID NO: 106 - DNA, 17 bp, Artificial Sequence
  probe HPA-3bd CR
gctggggctgggcagcc

SEQ ID NO: 107 - DNA, 17 bp, Artificial Sequence
  probe HPA-3bd2 CR
ctggggctgggcagccc

SEQ ID NO: 108 - DNA, 17 bp, Artificial Sequence
  probe HPA-3be CR
tggggctgggcagcccc

SEQ ID NO: 109 - DNA, 18 bp, Artificial Sequence
  probe HPA-4aa
gccacccagatgcgaaag

SEQ ID NO: 110 - DNA, 18 bp, Artificial Sequence
  probe HPA-4ab
cacccagatgcgaaagct

SEQ ID NO: 111 - DNA, 18 bp, Artificial Sequence
  probe HPA-4ac
cccagatgcgaaagctca

SEQ ID NO: 112 - DNA, 18 bp, Artificial Sequence
  probe HPA-4ad
cagatgcgaaagctcacc

SEQ ID NO: 113 - DNA, 18 bp, Artificial Sequence
  probe HPA-4ae
gatgcgaaagctcaccag

SEQ ID NO: 114 - DNA, 18 bp, Artificial Sequence
  probe HPA-4aa CR
ctggtgagctttcgcatc

SEQ ID NO: 115 - DNA, 18 bp, Artificial Sequence
  probe HPA-4ab CR
ggtgagctttcgcatctg

SEQ ID NO: 116 - DNA, 18 bp, Artificial Sequence
  probe HPA-4ac CR
tgagctttcgcatctggg

SEQ ID NO: 117 - DNA, 18 bp, Artificial Sequence
  probe HPA-4ad CR
agctttcgcatctgggtg

SEQ ID NO: 118 - DNA, 18 bp, Artificial Sequence
  probe HPA-4ae CR
ctttcgcatctgggtggc

SEQ ID NO: 119 - DNA, 18 bp, Artificial Sequence
  probe HPA-4ba
gccacccagatgcaaaag

SEQ ID NO: 120 - DNA, 19 bp, Artificial Sequence
  probe HPA-4bb
ccacccagatgcaaaagct

SEQ ID NO: 121 - DNA, 20 bp, Artificial Sequence
  probe HPA-4bc
acccagatgcaaaagctcac

SEQ ID NO: 122 - DNA, 19 bp, Artificial Sequence
  probe HPA-4bd
cagatgcaaaagctcacca

SEQ ID NO: 123 - DNA, 21 bp, Artificial Sequence
  probe HPA-4be
gatgcaaaagctcaccagtaa

SEQ ID NO: 124 - DNA, 21 bp, Artificial Sequence
  probe HPA-4ba CR
ttactggtgagcttttgcatc

SEQ ID NO: 125 - DNA, 19 bp, Artificial Sequence
  probe HPA-4bb CR
tggtgagcttttgcatctg

SEQ ID NO: 126 - DNA, 20 bp, Artificial Sequence
  probe HPA-4bc CR
gtgagcttttgcatctgggt

SEQ ID NO: 127 - DNA, 19 bp, Artificial Sequence
  probe HPA-4bd CR
agcttttgcatctgggtgg

SEQ ID NO: 128 - DNA, 18 bp, Artificial Sequence
  probe HPA-4be CR
cttttgcatctgggtggc

SEQ ID NO: 129 - DNA, 27 bp, Artificial Sequence
  probe HPA-5aa
gagtctacctgtttactatcaaagagg

SEQ ID NO: 130 - DNA, 28 bp, Artificial Sequence
  probe HPA-5ab
agtctacctgtttactatcaaagaggta

SEQ ID NO: 131 - DNA, 28 bp, Artificial Sequence
  probe HPA-5ac
gtctacctgtttactatcaaagaggtaa

SEQ ID NO: 132 - DNA, 28 bp, Artificial Sequence
  probe HPA-5ad
ctacctgtttactatcaaagaggtaaaa

SEQ ID NO: 133 - DNA, 27 bp, Artificial Sequence
  probe HPA-5ae
acctgtttactatcaaagaggtaaaaa

SEQ ID NO: 134 - DNA, 27 bp, Artificial Sequence
  probe HPA-5aa CR
tttttacctctttgatagtaaacaggt

SEQ ID NO: 135 - DNA, 28 bp, Artificial Sequence
  probe HPA-5ab CR
ttttacctctttgatagtaaacaggtag

SEQ ID NO: 136 - DNA, 28 bp, Artificial Sequence
  probe HPA-5ac CR
ttacctctttgatagtaaacaggtagac

SEQ ID NO: 137 - DNA, 28 bp, Artificial Sequence
  probe HPA-5ad CR
tacctctttgatagtaaacaggtagact

SEQ ID NO: 138 - DNA, 27 bp, Artificial Sequence
  probe HPA-5ae CR
cctctttgatagtaaacaggtagactc

SEQ ID NO: 139 - DNA, 27 bp, Artificial Sequence
  probe HPA-5ba
gagtctacctgtttactatcaaaaagg

SEQ ID NO: 140 - DNA, 28 bp, Artificial Sequence
  probe HPA-5bb
agtctacctgtttactatcaaaaaggta

SEQ ID NO: 141 - DNA, 28 bp, Artificial Sequence
  probe HPA-5bc
gtctacctgtttactatcaaaaaggtaa

SEQ ID NO: 142 - DNA, 28 bp, Artificial Sequence
  probe HPA-5ad
ctacctgtttactatcaaaaaggtaaaa

SEQ ID NO: 143 - DNA, 27 bp, Artificial Sequence
  probe HPA-5be
acctgtttactatcaaaaaggtaaaaa

SEQ ID NO: 144 - DNA, 27 bp, Artificial Sequence
  probe HPA-5ba CR
tttttacctttttgatagtaaacaggt

SEQ ID NO: 145 - DNA, 28 bp, Artificial Sequence
  probe HPA-5bb CR
ttttacctttttgatagtaaacaggtag

SEQ ID NO: 146 - DNA, 28 bp, Artificial Sequence
  probe HPA-5bc CR
ttacctttttgatagtaaacaggtagac

SEQ ID NO: 147 - DNA, 28 bp, Artificial Sequence
  probe HPA-5bd CR
tacctttttgatagtaaacaggtagact

SEQ ID NO: 148 - DNA, 27 bp, Artificial Sequence
  probe HPA-5be CR
cctttttgatagtaaacaggtagactc

SEQ ID NO: 149 - DNA, 29 bp, Artificial Sequence
  probe Gov-aa
ttattatcttgacttcagttacaggattt

SEQ ID NO: 150 - DNA, 26 bp, Artificial Sequence
  probe Gov-ab
tcttgacttcagttacaggatttacc

SEQ ID NO: 151 - DNA, 25 bp, Artificial Sequence
  probe Gov-ac
tgacttcagttacaggatttaccaa

SEQ ID NO: 152 - DNA, 26 bp, Artificial Sequence
  probe Gov-ad
cttcagttacaggatttaccaagaat

SEQ ID NO: 153 - DNA, 26 bp, Artificial Sequence
  probe Gov-ae
cagttacaggatttaccaagaatttg

SEQ ID NO: 154 - DNA, 26 bp, Artificial Sequence
  probe Gov-aa CR
caaattcttggtaaatcctgtaactg

SEQ ID NO: 155 - DNA, 26 bp, Artificial Sequence
  probe Gov-ab CR
attcttggtaaatcctgtaactgaag

SEQ ID NO: 156 - DNA, 25 bp, Artificial Sequence
  probe Gov-ac CR
tggtaaatcctgtaactgaagtcaa

SEQ ID NO: 157 - DNA, 26 bp, Artificial Sequence
  probe Gov-ad CR
ggtaaatcctgtaactgaagtcaaga

SEQ ID NO: 158 - DNA, 29 bp, Artificial Sequence
  probe Gov-ae CR
aaatcctgtaactgaagtcaagataataa

SEQ ID NO: 159 - DNA, 24 bp, Artificial Sequence
  probe Gov-ba
tatcttgacttcagttccaggatt

SEQ ID NO: 160 - DNA, 24 bp, Artificial Sequence
  probe Gov-bb
cttgacttcagttccaggatttac

SEQ ID NO: 161 - DNA, 23 bp, Artificial Sequence
  probe Gov-bc
gacttcagttccaggatttacca

SEQ ID NO: 162 - DNA, 22 bp, Artificial Sequence
  probe Gov-bd
ttcagttccaggatttaccaag

SEQ ID NO: 163 - DNA, 24 bp, Artificial Sequence
  probe Gov-be
cagttccaggatttaccaagaatt

SEQ ID NO: 164 - DNA, 24 bp, Artificial Sequence
  probe Gov-ba CR
aattcttggtaaatcctggaactg

SEQ ID NO: 165 - DNA, 22 bp, Artificial Sequence
  probe Gov-bb CR
cttggtaaatcctggaactgaa

SEQ ID NO: 166 - DNA, 23 bp, Artificial Sequence
  probe Gov-bc CR
ggtaaatcctggaactgaagtca

SEQ ID NO: 167 - DNA, 24 bp, Artificial Sequence
  probe Gov-bd CR
gtaaatcctggaactgaagtcaag

SEQ ID NO: 168 - DNA, 24 bp, Artificial Sequence
  probe Gov-be CR
aatcctggaactgaagtcaagata

SEQ ID NO: 169 - DNA, 20 bp, Artificial Sequence
  probe a33
tcagccctatcgcaggatgt

SEQ ID NO: 170 - DNA, 20 bp, Artificial Sequence
  probe a35
gagacacttgacagtagcca

SEQ ID NO: 171 - DNA, 20 bp, Artificial Sequence
  probe a38
ggcagggcacctcagtttat

SEQ ID NO: 172 - DNA, 20 bp, Artificial Sequence
  probe a42
tcaccagccagactgtgtag

SEQ ID NO: 173 - DNA, 20 bp, Artificial Sequence
  probe a43
cttcacgcaagttgtccaga

SEQ ID NO: 174 - DNA, 20 bp, Artificial Sequence
  oligo CS05
gtcctgacttctagctcatg

SEQ ID NO: 175 - DNA, 41 bp, Artificial Sequence
  Fyx-left
gccgcgaattcactagtgtcatgcttttcagacctctcttc

SEQ ID NO: 176 - DNA, 36 bp, Artificial Sequence
  Fyx-right
ggccgcgggaattcgattcaagacgggcaccacaat

SEQ ID NO: 177 - DNA, 38 bp, Artificial Sequence
  KEL6/7-left
gccgcgaattcactagtggcagcaccaaccctatgttc

SEQ ID NO: 178 - DNA, 37 bp, Artificial Sequence
  KEL6/7-right
ggccgcgggaattcgatttcaggcacaggtgagcttc

SEQ ID NO: 179 - DNA, 36 bp, Artificial Sequence
  RHDex3-left
gccgcgaattcactagtgtcctggctctccctctct

SEQ ID NO: 180 - DNA, 37 bp, Artificial Sequence
  RHCEex3-right
ggccgcgggaattcgatttttttcaaaaccccggaag

SEQ ID NO: 181 - DNA, 41 bp, Artificial Sequence
  RhDVI-left (fw)
ggccgcgggaattcgattctttgaattaagcacttcacaga

SEQ ID NO: 182 - DNA, 38 bp, Artificial Sequence
  RhDVI-right (rev)
gccgcgaattcactagtggccagaatcacactcctgct

SEQ ID NO: 183 - DNA, 39 bp, Artificial Sequence
  Ss-left
gccgcgaattcactagtgtttttctttgcacatgtcttt

SEQ ID NO: 184 - DNA, 42 bp, Artificial Sequence
  Ss-right
ggccgcgggaattcgatttctttgtctttacaatttcgtgtg

SEQ ID NO: 185 - DNA, 38 bp, Artificial Sequence
  DO-left
ggccgcgggaattcgatttgatccctccctatgagctg

SEQ ID NO: 186 - DNA, 41 bp, Artificial Sequence
  DO-right
gccgcgaattcactagtgttatatgtgctcaggttcccagt

SEQ ID NO: 187 - DNA, 37 bp, Artificial Sequence
  Colton-left
gccgcgaattcactagtggccacgaccctctttgtct

SEQ ID NO: 188 - DNA, 38 bp, Artificial Sequence
  Colton-right
ggccgcgggaattcgatttacatgagggcacggaagat

SEQ ID NO: 189 - DNA, 38 bp, Artificial Sequence
  Diego-left
gccgcgaattcactagtgacttattcacgggcatccag

SEQ ID NO: 190 - DNA, 38 bp, Artificial Sequence
  Diego-right
ggccgcgggaattcgattaagctccacgttcctgaaga

SEQ ID NO: 191 - DNA, 17 bp, Artificial Sequence
  Co.a.1
aacaaccagacggcggt

SEQ ID NO: 192 - DNA, 17 bp, Artificial Sequence
  Co.a.2
accagacggcggtccag

SEQ ID NO: 193 - DNA, 17 bp, Artificial Sequence
  Co.a.3
cagacggcggtccagga

SEQ ID NO: 194 - DNA, 18 bp, Artificial Sequence
  Co.a.4
gacggcggtccaggacaa

SEQ ID NO: 195 - DNA, 17 bp, Artificial Sequence
  Co.a.5
cggcggtccaggacaac

SEQ ID NO: 196 - DNA, 18 bp, Artificial Sequence
  Co.a.1.cr
gttgtcctggaccgccgt

SEQ ID NO: 197 - DNA, 17 bp, Artificial Sequence
  Co.a.2.cr
tcctggaccgccgtctg

SEQ ID NO: 198 - DNA, 17 bp, Artificial Sequence
  Co.a.3.cr
ctggaccgccgtctggt

SEQ ID NO: 199 - DNA, 17 bp, Artificial Sequence
  Co.a.4.cr
ggaccgccgtctggttg

SEQ ID NO: 200 - DNA, 17 bp, Artificial Sequence
  Co.a.5.cr
accgccgtctggttgtt

SEQ ID NO: 201 - DNA, 19 bp, Artificial Sequence
  Co.b.1
ggaacaaccagacggtggt

SEQ ID NO: 202 - DNA, 20 bp, Artificial Sequence
  Co.b.2
acaaccagacggtggtccag

SEQ ID NO: 203 - DNA, 19 bp, Artificial Sequence
  Co.b.3
accagacggtggtccagga

SEQ ID NO: 204 - DNA, 20 bp, Artificial Sequence
  Co.b.4
gacggtggtccaggacaacg

SEQ ID NO: 205 - DNA, 18 bp, Artificial Sequence
  Co.b.5
cggtggtccaggacaacg

SEQ ID NO: 206 - DNA, 19 bp, Artificial Sequence
  Co.b.1.cr
cgttgtcctggaccaccgt

SEQ ID NO: 207 - DNA, 20 bp, Artificial Sequence
  Co.b.2.cr
ttgtcctggaccaccgtctg

SEQ ID NO: 208 - DNA, 20 bp, Artificial Sequence
  Co.b.3.cr
ctggaccaccgtctggttgt

SEQ ID NO: 209 - DNA, 20 bp, Artificial Sequence
  Co.b.4.cr
ggaccaccgtctggttgttc

SEQ ID NO: 210 - DNA, 19 bp, Artificial Sequence
  Co.b.5.cr
accaccgtctggttgttcc

SEQ ID NO: 211 - DNA, 17 bp, Artificial Sequence
  Di.a.1
gtgaagtccacgccggc

SEQ ID NO: 212 - DNA, 17 bp, Artificial Sequence
  Di.a.2
gaagtccacgccggcct

SEQ ID NO: 213 - DNA, 17 bp, Artificial Sequence
  Di.a.3
agtccacgccggcctcc

SEQ ID NO: 214 - DNA, 17 bp, Artificial Sequence
  Di.a.4
tccacgccggcctccct

SEQ ID NO: 215 - DNA, 18 bp, Artificial Sequence
  Di.a.5
acgccggcctccctggcc

SEQ ID NO: 216 - DNA, 17 bp, Artificial Sequence
  Di.a.1.cr
gccagggaggccggcgt

SEQ ID NO: 217 - DNA, 17 bp, Artificial Sequence
  Di.a.2.cr
agggaggccggcgtgga

SEQ ID NO: 218 - DNA, 17 bp, Artificial Sequence
  Di.a.3.cr
ggaggccggcgtggact

SEQ ID NO: 219 - DNA, 17 bp, Artificial Sequence
  Di.a.4.cr
aggccggcgtggacttc

SEQ ID NO: 220 - DNA, 17 bp, Artificial Sequence
  Di.a.5.cr
ggccggcgtggacttca

SEQ ID NO: 221 - DNA, 18 bp, Artificial Sequence
  Di.b.1
ggtgaagtccacgctggc

SEQ ID NO: 222 - DNA, 19 bp, Artificial Sequence
  Di.b.2
gtgaagtccacgctggcct

SEQ ID NO: 223 - DNA, 20 bp, Artificial Sequence
  Di.b.3
tgaagtccacgctggcctcc

SEQ ID NO: 224 - DNA, 21 bp, Artificial Sequence
  Di.b.4
gaagtccacgctggcctccct

SEQ ID NO: 225 - DNA, 19 bp, Artificial Sequence
  Di.b.5
acgctggcctccctggccc

SEQ ID NO: 226 - DNA, 18 bp, Artificial Sequence
  Di.b.1.cr
ggccagggaggccagcgt

SEQ ID NO: 227 - DNA, 18 bp, Artificial Sequence
  Di.b.2.cr
cagggaggccagcgtgga

SEQ ID NO: 228 - DNA, 19 bp, Artificial Sequence
  Di.b.3.cr
agggaggccagcgtggact

SEQ ID NO: 229 - DNA, 19 bp, Artificial Sequence
  Di.b.4.cr
ggaggccagcgtggacttc

SEQ ID NO: 230 - DNA, 19 bp, Artificial Sequence
  Di.b.5.cr
ggccagcgtggacttcacc

SEQ ID NO: 231 - DNA, 18 bp, Artificial Sequence
  Wr.a.1
tgggcttgcgttccaagt

SEQ ID NO: 232 - DNA, 19 bp, Artificial Sequence
  Wr.a.2
ggcttgcgttccaagtttc

SEQ ID NO: 233 - DNA, 19 bp, Artificial Sequence
  Wr.a.3
ttgcgttccaagtttccca

SEQ ID NO: 234 - DNA, 19 bp, Artificial Sequence
  Wr.a.4
cgttccaagtttcccatct

SEQ ID NO: 235 - DNA, 19 bp, Artificial Sequence
  Wr.a.5
tccaagtttcccatctgga

SEQ ID NO: 236 - DNA, 19 bp, Artificial Sequence
  Wr.a.1 CR
tccagatgggaaacttgga

SEQ ID NO: 237 - DNA, 19 bp, Artificial Sequence
  Wr.a.2 CR
agatgggaaacttggaacg

SEQ ID NO: 238 - DNA, 19 bp, Artificial Sequence
  Wr.a.3 CR
tgggaaacttggaacgcaa

SEQ ID NO: 239 - DNA, 19 bp, Artificial Sequence
  Wr.a.4 CR
gaaacttggaacgcaagcc

SEQ ID NO: 240 - DNA, 18 bp, Artificial Sequence
  Wr.a.5 CR
acttggaacgcaagccca

SEQ ID NO: 241 - DNA, 18 bp, Artificial Sequence
  Wr.b.1
gggcttgcgttccgagtt

SEQ ID NO: 242 - DNA, 18 bp, Artificial Sequence
  Wr.b.2
gcttgcgttccgagtttc

SEQ ID NO: 243 - DNA, 18 bp, Artificial Sequence
  Wr.b.3
ttgcgttccgagtttccc

SEQ ID NO: 244 - DNA, 18 bp, Artificial Sequence
  Wr.b.4
cgttccgagtttcccatc

SEQ ID NO: 245 - DNA, 18 bp, Artificial Sequence
  Wr.b.5
tccgagtttcccatctgg

SEQ ID NO: 246 - DNA, 18 bp, Artificial Sequence
  Wr.b.1 CR
ccagatgggaaactcgga

SEQ ID NO: 247 - DNA, 18 bp, Artificial Sequence
  Wr.b.2 CR
gatgggaaactcggaacg

SEQ ID NO: 248 - DNA, 18 bp, Artificial Sequence
  Wr.b.3 CR
gggaaactcggaacgcaa

SEQ ID NO: 249 - DNA, 18 bp, Artificial Sequence
  Wr.b.4 CR
gaaactcggaacgcaagc

SEQ ID NO: 250 - DNA, 18 bp, Artificial Sequence
  Wr.b.5 CR
aactcggaacgcaagccc

SEQ ID NO: 251 - DNA, 19 bp, Artificial Sequence
  Do.a.1
taccacccaagaggaaact

SEQ ID NO: 252 - DNA, 19 bp, Artificial Sequence
  Do.a.2
ccacccaagaggaaactgg

SEQ ID NO: 253 - DNA, 20 bp, Artificial Sequence
  Do.a.3
acccaagaggaaactggttg

SEQ ID NO: 254 - DNA, 19 bp, Artificial Sequence
  Do.a.4
caagaggaaactggttgca

SEQ ID NO: 255 - DNA, 20 bp, Artificial Sequence
  Do.a.5
aggaaactggttgcagttga

SEQ ID NO: 256 - DNA, 20 bp, Artificial Sequence
  Do.a.6 cr
ctcaactgcaaccagtttcc

SEQ ID NO: 257 - DNA, 20 bp, Artificial Sequence
  Do.a.7 cr
caactgcaaccagtttcctc

SEQ ID NO: 258 - DNA, 20 bp, Artificial Sequence
  Do.a.8 cr
tgcaaccagtttcctcttgg

SEQ ID NO: 259 - DNA, 20 bp, Artificial Sequence
  Do.a.9 cr
accagtttcctcttgggtgg

SEQ ID NO: 260 - DNA, 20 bp, Artificial Sequence
  Do.a.10 cr
cagtttcctcttgggtggta

SEQ ID NO: 261 - DNA, 19 bp, Artificial Sequence
  Do.b.1
taccacccaagaggagact

SEQ ID NO: 262 - DNA, 19 bp, Artificial Sequence
  Do.b.2
ccacccaagaggagactgg

SEQ ID NO: 263 - DNA, 20 bp, Artificial Sequence
  Do.b.3
acccaagaggagactggttg

SEQ ID NO: 264 - DNA, 19 bp, Artificial Sequence
  Do.b.4
caagaggagactggttgca

SEQ ID NO: 265 - DNA, 20 bp, Artificial Sequence
  Do.b.5
aggagactggttgcagttga

SEQ ID NO: 266 - DNA, 20 bp, Artificial Sequence
  Do.b.6 cr
ctcaactgcaaccagtctcc

SEQ ID NO: 267 - DNA, 20 bp, Artificial Sequence
  Do.b.7 cr
caactgcaaccagtctcctc

SEQ ID NO: 268 - DNA, 20 bp, Artificial Sequence
  Do.b.8 cr
tgcaaccagtctcctcttgg

SEQ ID NO: 269 - DNA, 20 bp, Artificial Sequence
  Do.b.9 cr
accagtctcctcttgggtgg

SEQ ID NO: 270 - DNA, 19 bp, Artificial Sequence
  Do.b.10 cr
cagtctcctcttgggtggt

SEQ ID NO: 271 - DNA, 20 bp, Artificial Sequence
  Jo.a.pos.1
ccccagaacatgactaccac

SEQ ID NO: 272 - DNA, 21 bp, Artificial Sequence
  Jo.a.pos.2
ccagaacatgactaccacaca

SEQ ID NO: 273 - DNA, 22 bp, Artificial Sequence
  Jo.a.pos.3
agaacatgactaccacacacgc

SEQ ID NO: 274 - DNA, 21 bp, Artificial Sequence
  Jo.a.pos.4
catgactaccacacacgctgt

SEQ ID NO: 275 - DNA, 19 bp, Artificial Sequence
  Jo.a.pos.5
actaccacacacgctgtgg

SEQ ID NO: 276 - DNA, 20 bp, Artificial Sequence
  Jo.a.pos.1 cr
gccacagcgtgtgtggtagt

SEQ ID NO: 277 - DNA, 21 bp, Artificial Sequence
  Jo.a.pos.2 cr
acagcgtgtgtggtagtcatg

SEQ ID NO: 278 - DNA, 21 bp, Artificial Sequence
  Jo.a.pos.3 cr
agcgtgtgtggtagtcatgtt

SEQ ID NO: 279 - DNA, 22 bp, Artificial Sequence
  Jo.a.pos.4 cr
cgtgtgtggtagtcatgttctg

SEQ ID NO: 280 - DNA, 20 bp, Artificial Sequence
  Jo.a.pos.5 cr
gtggtagtcatgttctgggg

SEQ ID NO: 281 - DNA, 23 bp, Artificial Sequence
  Jo.a.neg.1
ctaccccagaacatgactatcac

SEQ ID NO: 282 - DNA, 22 bp, Artificial Sequence
  Jo.a.neg.2
cccagaacatgactatcacaca

SEQ ID NO: 283 - DNA, 23 bp, Artificial Sequence
  Jo.a.neg.3
cagaacatgactatcacacacgc

SEQ ID NO: 284 - DNA, 22 bp, Artificial Sequence
  Jo.a.neg.4
catgactatcacacacgctgtg

SEQ ID NO: 285 - DNA, 20 bp, Artificial Sequence
  Jo.a.neg.5
actatcacacacgctgtggc

SEQ ID NO: 286 - DNA, 24 bp, Artificial Sequence
  Jo.a.neg.1 cr
aatagccacagcgtgtgtgatagt

SEQ ID NO: 287 - DNA, 22 bp, Artificial Sequence
  Jo.a.neg.2 cr
cacagcgtgtgtgatagtcatg

SEQ ID NO: 288 - DNA, 22 bp, Artificial Sequence
  Jo.a.neg.3 cr
cagcgtgtgtgatagtcatgtt

SEQ ID NO: 289 - DNA, 23 bp, Artificial Sequence
  Jo.a.neg.4 cr
cgtgtgtgatagtcatgttctgg

SEQ ID NO: 290 - DNA, 23 bp, Artificial Sequence
  Jo.a.neg.5 cr
gtgatagtcatgttctggggtag

SEQ ID NO: 291 - DNA, 21 bp, Artificial Sequence
  Fy.A.1
ccagatggagactatggtgcc

SEQ ID NO: 292 - DNA, 20 bp, Artificial Sequence
  Fy.A.2
atggagactatggtgccaac

SEQ ID NO: 293 - DNA, 21 bp, Artificial Sequence
  Fy.A.3
ggagactatggtgccaacctg

SEQ ID NO: 294 - DNA, 20 bp, Artificial Sequence
  Fy.A.4
gactatggtgccaacctgga

SEQ ID NO: 295 - DNA, 19 bp, Artificial Sequence
  Fy.A.5
tatggtgccaacctggaag

SEQ ID NO: 296 - DNA, 19 bp, Artificial Sequence
  Fy.A.1.cr
cttccaggttggcaccata

SEQ ID NO: 297 - DNA, 20 bp, Artificial Sequence
  Fy.A.2.cr
tccaggttggcaccatagtc

SEQ ID NO: 298 - DNA, 20 bp, Artificial Sequence
  Fy.A.3.cr
aggttggcaccatagtctcc

SEQ ID NO: 299 - DNA, 20 bp, Artificial Sequence
  Fy.A.4.cr
gttggcaccatagtctccat

SEQ ID NO: 300 - DNA, 20 bp, Artificial Sequence
  Fy.A.5.cr
gcaccatagtctccatctgg

SEQ ID NO: 301 - DNA, 22 bp, Artificial Sequence
  Fy.B.1
cccagatggagactatgatgcc

SEQ ID NO: 302 - DNA, 21 bp, Artificial Sequence
  Fy.B.2
gatggagactatgatgccaac

SEQ ID NO: 303 - DNA, 22 bp, Artificial Sequence
  Fy.B.3
tggagactatgatgccaacctg

SEQ ID NO: 304 - DNA, 21 bp, Artificial Sequence
  Fy.B.4
gactatgatgccaacctggaa

SEQ ID NO: 305 - DNA, 20 bp, Artificial Sequence
  Fy.B.5
tatgatgccaacctggaagc

SEQ ID NO: 306 - DNA, 20 bp, Artificial Sequence
  Fy.B.1.cr
gcttccaggttggcatcata

SEQ ID NO: 307 - DNA, 21 bp, Artificial Sequence
  Fy.B.2.cr
ttccaggttggcatcatagtc

SEQ ID NO: 308 - DNA, 21 bp, Artificial Sequence
  Fy.B.3.cr
caggttggcatcatagtctcc

SEQ ID NO: 309 - DNA, 21 bp, Artificial Sequence
  Fy.B.4.cr
gttggcatcatagtctccatc

SEQ ID NO: 310 - DNA, 21 bp, Artificial Sequence
  Fy.B.5.cr
gcatcatagtctccatctggg

SEQ ID NO: 311 - DNA, 20 bp, Artificial Sequence
  Fy.GATA.normal.1
agtccttggctcttatcttg

SEQ ID NO: 312 - DNA, 22 bp, Artificial Sequence
  Fy.GATA.normal.2
agtccttggctcttatcttgga

SEQ ID NO: 313 - DNA, 21 bp, Artificial Sequence
  Fy.GATA.normal.3
cttggctcttatcttggaagc

SEQ ID NO: 314 - DNA, 22 bp, Artificial Sequence
  Fy.GATA.normal.4
gctcttatcttggaagcacagg

SEQ ID NO: 315 - DNA, 22 bp, Artificial Sequence
  Fy.GATA.normal.5
cttatcttggaagcacaggcgc

SEQ ID NO: 316 - DNA, 20 bp, Artificial Sequence
  Fy.GATA.normal.1.cr
gcctgtgcttccaagataag

SEQ ID NO: 317 - DNA, 20 bp, Artificial Sequence
  Fy.GATA.normal.2.cr
tgtgcttccaagataagagc

SEQ ID NO: 318 - DNA, 20 bp, Artificial Sequence
  Fy.GATA.normal.3.cr
tgcttccaagataagagcca

SEQ ID NO: 319 - DNA, 21 bp, Artificial Sequence
  Fy.GATA.normal.4.cr
ttccaagataagagccaagga

SEQ ID NO: 320 - DNA, 20 bp, Artificial Sequence
  Fy.GATA.normal.5.cr
caagataagagccaaggact

SEQ ID NO: 321 - DNA, 18 bp, Artificial Sequence
  Fy.GATA.mut.1
tccttggctcttaccttg

SEQ ID NO: 322 - DNA, 20 bp, Artificial Sequence
  Fy.GATA.mut.2
tccttggctcttaccttgga

SEQ ID NO: 323 - DNA, 19 bp, Artificial Sequence
  Fy.GATA.mut.3
tggctcttaccttggaagc

SEQ ID NO: 324 - DNA, 21 bp, Artificial Sequence
  Fy.GATA.mut.4
gctcttaccttggaagcacag

SEQ ID NO: 325 - DNA, 21 bp, Artificial Sequence
  Fy.GATA.mut.5
cttaccttggaagcacaggcg

SEQ ID NO: 326 - DNA, 19 bp, Artificial Sequence
  Fy.GATA.mut.1.cr
cctgtgcttccaaggtaag

SEQ ID NO: 327 - DNA, 19 bp, Artificial Sequence
  Fy.GATA.mut.2.cr
gtgcttccaaggtaagagc

SEQ ID NO: 328 - DNA, 19 bp, Artificial Sequence
  Fy.GATA.mut.3.cr
gcttccaaggtaagagcca

SEQ ID NO: 329 - DNA, 20 bp, Artificial Sequence
  Fy.GATA.mut.4.cr
ttccaaggtaagagccaagg

SEQ ID NO: 330 - DNA, 18 bp, Artificial Sequence
  Fy.GATA.mut.5.cr
caaggtaagagccaagga

SEQ ID NO: 331 - DNA, 21 bp, Artificial Sequence
  Fy.X.(b).1
ttttcagacctctcttccgct

SEQ ID NO: 332 - DNA, 19 bp, Artificial Sequence
  Fy.X.(b).2
agacctctcttccgctggc

SEQ ID NO: 333 - DNA, 18 bp, Artificial Sequence
  Fy.X.(b).3
ctctcttccgctggcagc

SEQ ID NO: 334 - DNA, 18 bp, Artificial Sequence
  Fy.X.(b).4
ctcttccgctggcagctc

SEQ ID NO: 335 - DNA, 18 bp, Artificial Sequence
  Fy.X.(b).5
cttccgctggcagctctg

SEQ ID NO: 336 - DNA, 17 bp, Artificial Sequence
  Fy.X.(b).1.cr
cagagctgccagcggaa

SEQ ID NO: 337 - DNA, 17 bp, Artificial Sequence
  Fy.X.(b).2.cr
agctgccagcggaagag

SEQ ID NO: 338 - DNA, 18 bp, Artificial Sequence
  Fy.X.(b).3.cr
ctgccagcggaagagagg

SEQ ID NO: 339 - DNA, 18 bp, Artificial Sequence
  Fy.X.(b).4.cr
gccagcggaagagaggtc

SEQ ID NO: 340 - DNA, 18 bp, Artificial Sequence
  Fy.X.(b).5.cr
cagcggaagagaggtctg

SEQ ID NO: 341 - DNA, 23 bp, Artificial Sequence
  Fy.X.1
gcttttcagacctctcttctgct

SEQ ID NO: 342 - DNA, 21 bp, Artificial Sequence
  Fy.X.2
tcagacctctcttctgctggc

SEQ ID NO: 343 - DNA, 20 bp, Artificial Sequence
  Fy.X.3
acctctcttctgctggcagc

SEQ ID NO: 344 - DNA, 20 bp, Artificial Sequence
  Fy.X.4
ctcttctgctggcagctctg

SEQ ID NO: 345 - DNA, 19 bp, Artificial Sequence
  Fy.X.5
cttctgctggcagctctgc

SEQ ID NO: 346 - DNA, 18 bp, Artificial Sequence
  Fy.X.1.cr
gcagagctgccagcagaa

SEQ ID NO: 347 - DNA, 20 bp, Artificial Sequence
  Fy.X.2.cr
gagctgccagcagaagagag

SEQ ID NO: 348 - DNA, 20 bp, Artificial Sequence
  Fy.X.3.cr
agctgccagcagaagagagg

SEQ ID NO: 349 - DNA, 20 bp, Artificial Sequence
  Fy.X.4.cr
gccagcagaagagaggtctg

SEQ ID NO: 350 - DNA, 21 bp, Artificial Sequence
  Fy.X.5.cr
cagcagaagagaggtctgaaa

SEQ ID NO: 351 - DNA, 18 bp, Artificial Sequence
  Jk.a.1
cagccccatttgaggaca

SEQ ID NO: 352 - DNA, 20 bp, Artificial Sequence
  Jk.a.2
gccccatttgaggacatcta

SEQ ID NO: 353 - DNA, 22 bp, Artificial Sequence
  Jk.a.3
ccatttgaggacatctactttg

SEQ ID NO: 354 - DNA, 22 bp, Artificial Sequence
  Jk.a.4
atttgaggacatctactttgga

SEQ ID NO: 355 - DNA, 22 bp, Artificial Sequence
  Jk.a.5
gaggacatctactttggactct

SEQ ID NO: 356 - DNA, 23 bp, Artificial Sequence
  Jk.a.1.cr
cagagtccaaagtagatgtcctc

SEQ ID NO: 357 - DNA, 23 bp, Artificial Sequence
  Jk.a.2.cr
agtccaaagtagatgtcctcaaa

SEQ ID NO: 358 - DNA, 22 bp, Artificial Sequence
  Jk.a.3.cr
aaagtagatgtcctcaaatggg

SEQ ID NO: 359 - DNA, 20 bp, Artificial Sequence
  Jk.a.4.cr
tagatgtcctcaaatggggc

SEQ ID NO: 360 - DNA, 19 bp, Artificial Sequence
  Jk.a.5.cr
atgtcctcaaatggggctg

SEQ ID NO: 361 - DNA, 19 bp, Artificial Sequence
  Jk.b.1
tcagccccatttgagaaca

SEQ ID NO: 362 - DNA, 20 bp, Artificial Sequence
  Jk.b.2
gccccatttgagaacatcta

SEQ ID NO: 363 - DNA, 23 bp, Artificial Sequence
  Jk.b.3
cccatttgagaacatctactttg

SEQ ID NO: 364 - DNA, 23 bp, Artificial Sequence
  Jk.b.4
atttgagaacatctactttggac

SEQ ID NO: 365 - DNA, 23 bp, Artificial Sequence
  Jk.b.5
gagaacatctactttggactctg

SEQ ID NO: 366 - DNA, 24 bp, Artificial Sequence
  Jk.b.1.cr
ccagagtccaaagtagatgttctc

SEQ ID NO: 367 - DNA, 25 bp, Artificial Sequence
  Jk.b.2.cr
agagtccaaagtagatgttctcaaa

SEQ ID NO: 368 - DNA, 23 bp, Artificial Sequence
  Jk.b.3.cr
ccaaagtagatgttctcaaatgg

SEQ ID NO: 369 - DNA, 22 bp, Artificial Sequence
  Jk.b.4.cr
agtagatgttctcaaatggggc

SEQ ID NO: 370 - DNA, 20 bp, Artificial Sequence
  Jk.b.5.cr
atgttctcaaatggggctga

SEQ ID NO: 371 - DNA, 23 bp, Artificial Sequence
  KEL1.1
tccttaaactttaaccgaatgct

SEQ ID NO: 372 - DNA, 24 bp, Artificial Sequence
  KEL1.2
ttaaactttaaccgaatgctgaga

SEQ ID NO: 373 - DNA, 24 bp, Artificial Sequence
  KEL1.3
aactttaaccgaatgctgagactt

SEQ ID NO: 374 - DNA, 21 bp, Artificial Sequence
  KEL1.4
aaccgaatgctgagacttctg

SEQ ID NO: 375 - DNA, 23 bp, Artificial Sequence
  KEL1.5
cgaatgctgagacttctgatgag

SEQ ID NO: 376 - DNA, 23 bp, Artificial Sequence
  KEL1.1 CR
actcatcagaagtctcagcattc

SEQ ID NO: 377 - DNA, 21 bp, Artificial Sequence
  KEL1.2 CR
tcagaagtctcagcattcggt

SEQ ID NO: 378 - DNA, 22 bp, Artificial Sequence
  KEL1.3 CR
aagtctcagcattcggttaaag

SEQ ID NO: 379 - DNA, 24 bp, Artificial Sequence
  KEL1.4 CR
tctcagcattcggttaaagtttaa

SEQ ID NO: 380 - DNA, 23 bp, Artificial Sequence
  KEL1.5 CR
agcattcggttaaagtttaagga

SEQ ID NO: 381 - DNA, 22 bp, Artificial Sequence
  KEL.2.1
ccttaaactttaaccgaacgct

SEQ ID NO: 382 - DNA, 21 bp, Artificial Sequence
  KEL.2.2
aactttaaccgaacgctgaga

SEQ ID NO: 383 - DNA, 22 bp, Artificial Sequence
  KEL.2.3
ctttaaccgaacgctgagactt

SEQ ID NO: 384 - DNA, 20 bp, Artificial Sequence
  KEL.2.4
aaccgaacgctgagacttct

SEQ ID NO: 385 - DNA, 21 bp, Artificial Sequence
  KEL.2.5
cgaacgctgagacttctgatg

SEQ ID NO: 386 - DNA, 21 bp, Artificial Sequence
  KEL.2.1 CR
tcatcagaagtctcagcgttc

SEQ ID NO: 387 - DNA, 20 bp, Artificial Sequence
  KEL.2.2 CR
cagaagtctcagcgttcggt

SEQ ID NO: 388 - DNA, 21 bp, Artificial Sequence
  KEL.2.3 CR
agtctcagcgttcggttaaag

SEQ ID NO: 389 - DNA, 21 bp, Artificial Sequence
  KEL.2.4 CR
tctcagcgttcggttaaagtt

SEQ ID NO: 390 - DNA, 22 bp, Artificial Sequence
  KEL.2.5 CR
agcgttcggttaaagtttaagg

SEQ ID NO: 391 - DNA, 21 bp, Artificial Sequence
  KEL.3.1
aatctccatcacttcatggct

SEQ ID NO: 392 - DNA, 20 bp, Artificial Sequence
  KEL.3.2
tccatcacttcatggctgtt

SEQ ID NO: 393 - DNA, 20 bp, Artificial Sequence
  KEL.3.3
atcacttcatggctgttcca

SEQ ID NO: 394 - DNA, 20 bp, Artificial Sequence
  KEL.3.4
acttcatggctgttccagtt

SEQ ID NO: 395 - DNA, 19 bp, Artificial Sequence
  KEL.3.5
tcatggctgttccagtttc

SEQ ID NO: 396 - DNA, 21 bp, Artificial Sequence
  KEL.3.1.cr
agaaactggaacagccatgaa

SEQ ID NO: 397 - DNA, 21 bp, Artificial Sequence
  KEL.3.2.cr
aactggaacagccatgaagtg

SEQ ID NO: 398 - DNA, 21 bp, Artificial Sequence
  KEL.3.3.cr
tggaacagccatgaagtgatg

SEQ ID NO: 399 - DNA, 20 bp, Artificial Sequence
  KEL.3.4.cr
acagccatgaagtgatggag

SEQ ID NO: 400 - DNA, 20 bp, Artificial Sequence
  KEL.3.5.cr
gccatgaagtgatggagatt

SEQ ID NO: 401 - DNA, 19 bp, Artificial Sequence
  KEL.4.1
tctccatcacttcacggct

SEQ ID NO: 402 - DNA, 19 bp, Artificial Sequence
  KEL.4.2
ccatcacttcacggctgtt

SEQ ID NO: 403 - DNA, 18 bp, Artificial Sequence
  KEL.4.3
cacttcacggctgttcca

SEQ ID NO: 404 - DNA, 18 bp, Artificial Sequence
  KEL.4.4
acttcacggctgttccag

SEQ ID NO: 405 - DNA, 18 bp, Artificial Sequence
  KEL.4.5
tcacggctgttccagttt

SEQ ID NO: 406 - DNA, 19 bp, Artificial Sequence
  KEL.4.1.cr
aaactggaacagccgtgaa

SEQ ID NO: 407 - DNA, 19 bp, Artificial Sequence
  KEL.4.2.cr
ctggaacagccgtgaagtg

SEQ ID NO: 408 - DNA, 20 bp, Artificial Sequence
  KEL.4.3.cr
ggaacagccgtgaagtgatg

SEQ ID NO: 409 - DNA, 18 bp, Artificial Sequence
  KEL.4.4.cr
acagccgtgaagtgatgg

SEQ ID NO: 410 - DNA, 18 bp, Artificial Sequence
  KEL.4.5.cr
gccgtgaagtgatggaga

SEQ ID NO: 411 - DNA, 23 bp, Artificial Sequence
  KEL.6.1
tactgcctgggggctgccccgcc

SEQ ID NO: 412 - DNA, 19 bp, Artificial Sequence
  KEL.6.2
tgggggctgccccgcctgt

SEQ ID NO: 413 - DNA, 17 bp, Artificial Sequence
  KEL.6.3
gctgccccgcctgtgac

SEQ ID NO: 414 - DNA, 18 bp, Artificial Sequence
  KEL.6.4
ctgccccgcctgtgacaa

SEQ ID NO: 415 - DNA, 17 bp, Artificial Sequence
  KEL.6.5
gccccgcctgtgacaac

SEQ ID NO: 416 - DNA, 17 bp, Artificial Sequence
  KEL.6.1.cr
gttgtcacaggcggggc

SEQ ID NO: 417 - DNA, 18 bp, Artificial Sequence
  KEL.6.2.cr
ttgtcacaggcggggcag

SEQ ID NO: 418 - DNA, 18 bp, Artificial Sequence
  KEL.6.3.cr
tcacaggcggggcagccc

SEQ ID NO: 419 - DNA, 19 bp, Artificial Sequence
  KEL.6.4.cr
acaggcggggcagccccca

SEQ ID NO: 420 - DNA, 17 bp, Artificial Sequence
  KEL.6.5.cr
aggcggggcagccccca

SEQ ID NO: 421 - DNA, 22 bp, Artificial Sequence
  KEL.7.1
actgcctgggggctgcctcgcc

SEQ ID NO: 422 - DNA, 21 bp, Artificial Sequence
  KEL.7.2
cctgggggctgcctcgcctgt

SEQ ID NO: 423 - DNA, 18 bp, Artificial Sequence
  KEL.7.3
ggctgcctcgcctgtgac

SEQ ID NO: 424 - DNA, 20 bp, Artificial Sequence
  KEL.7.4
ctgcctcgcctgtgacaacc

SEQ ID NO: 425 - DNA, 18 bp, Artificial Sequence
  KEL.7.5
gcctcgcctgtgacaacc

SEQ ID NO: 426 - DNA, 18 bp, Artificial Sequence
  KEL.7.1.cr
ggttgtcacaggcgaggc

SEQ ID NO: 427 - DNA, 20 bp, Artificial Sequence
  KEL.7.2.cr
ggttgtcacaggcgaggcag

SEQ ID NO: 428 - DNA, 21 bp, Artificial Sequence
  KEL.7.3.cr
ttgtcacaggcgaggcagccc

SEQ ID NO: 429 - DNA, 21 bp, Artificial Sequence
  KEL.7.4.cr
acaggcgaggcagcccccagg

SEQ ID NO: 430 - DNA, 19 bp, Artificial Sequence
  KEL.7.5.cr
aggcgaggcagcccccagg

SEQ ID NO: 431 - DNA, 17 bp, Artificial Sequence
  Lu.a.1
ggagctcgcccccgcct

SEQ ID NO: 432 - DNA, 17 bp, Artificial Sequence
  Lu.a.2
gctcgcccccgcctagc

SEQ ID NO: 433 - DNA, 17 bp, Artificial Sequence
  Lu.a.3
cgcccccgcctagcctc

SEQ ID NO: 434 - DNA, 17 bp, Artificial Sequence
  Lu.a.4
cccccgcctagcctcgg

SEQ ID NO: 435 - DNA, 17 bp, Artificial Sequence
  Lu.a.5
cccgcctagcctcggct

SEQ ID NO: 436 - DNA, 17 bp, Artificial Sequence
  Lu.a.1 CR
agccgaggctaggcggg

SEQ ID NO: 437 - DNA, 17 bp, Artificial Sequence
  Lu.a.2 CR
ccgaggctaggcggggg

SEQ ID NO: 438 - DNA, 17 bp, Artificial Sequence
  Lu.a.3 CR
gaggctaggcgggggcg

SEQ ID NO: 439 - DNA, 17 bp, Artificial Sequence
  Lu.a.4 CR
gctaggcgggggcgagc

SEQ ID NO: 440 - DNA, 17 bp, Artificial Sequence
  Lu.a.5 CR
aggcgggggcgagctcc

SEQ ID NO: 441 - DNA, 18 bp, Artificial Sequence
  Lu.b.1
gggagctcgcccccacct

SEQ ID NO: 442 - DNA, 19 bp, Artificial Sequence
  Lu.b.2
gagctcgcccccacctagc

SEQ ID NO: 443 - DNA, 19 bp, Artificial Sequence
  Lu.b.3
ctcgcccccacctagcctc

SEQ ID NO: 444 - DNA, 19 bp, Artificial Sequence
  Lu.b.4
cccccacctagcctcggct

SEQ ID NO: 445 - DNA, 19 bp, Artificial Sequence
  Lu.b.5
cccacctagcctcggctga

SEQ ID NO: 446 - DNA, 19 bp, Artificial Sequence
  Lu.b.1 CR
tcagccgaggctaggtggg

SEQ ID NO: 447 - DNA, 19 bp, Artificial Sequence
  Lu.b.2 CR
agccgaggctaggtggggg

SEQ ID NO: 448 - DNA, 19 bp, Artificial Sequence
  Lu.b.3 CR
gaggctaggtgggggcgag

SEQ ID NO: 449 - DNA, 19 bp, Artificial Sequence
  Lu.b.4 CR
gctaggtgggggcgagctc

SEQ ID NO: 450 - DNA, 18 bp, Artificial Sequence
  Lu.b.5 CR
aggtgggggcgagctccc

SEQ ID NO: 451 - DNA, 20 bp, Artificial Sequence
  M.1
gtgagcatatcagcatcaag

SEQ ID NO: 452 - DNA, 21 bp, Artificial Sequence
  M.2
atcagcatcaagtaccactgg

SEQ ID NO: 453 - DNA, 20 bp, Artificial Sequence
  M.3
catcaagtaccactggtgtg

SEQ ID NO: 454 - DNA, 20 bp, Artificial Sequence
  M.4
taccactggtgtggcaatgc

SEQ ID NO: 455 - DNA, 18 bp, Artificial Sequence
  M.5
ctggtgtggcaatgcaca

SEQ ID NO: 456 - DNA, 19 bp, Artificial Sequence
  M.1cr
tgtgcattgccacaccagt

SEQ ID NO: 457 - DNA, 20 bp, Artificial Sequence
  M.2.cr
gcattgccacaccagtggta

SEQ ID NO: 458 - DNA, 21 bp, Artificial Sequence
  M.3.cr
ccacaccagtggtacttgatg

SEQ ID NO: 459 - DNA, 19 bp, Artificial Sequence
  M.4.cr
accagtggtacttgatgct

SEQ ID NO: 460 - DNA, 20 bp, Artificial Sequence
  M.5.cr
cttgatgctgatatgctcac

SEQ ID NO: 461 - DNA, 21 bp, Artificial Sequence
  N.1
tgtgagcatatcagcattaag

SEQ ID NO: 462 - DNA, 23 bp, Artificial Sequence
  N.2
atcagcattaagtaccactgagg

SEQ ID NO: 463 - DNA, 21 bp, Artificial Sequence
  N.3
cattaagtaccactgaggtgg

SEQ ID NO: 464 - DNA, 19 bp, Artificial Sequence
  N.4
accactgaggtggcaatgc

SEQ ID NO: 465 - DNA, 20 bp, Artificial Sequence
  N.5
ctgaggtggcaatgcacact

SEQ ID NO: 466 - DNA, 20 bp, Artificial Sequence
  N.1.cr
gtgtgcattgccacctcagt

SEQ ID NO: 467 - DNA, 20 bp, Artificial Sequence
  N.2.cr
gcattgccacctcagtggta

SEQ ID NO: 468 - DNA, 22 bp, Artificial Sequence
  N.3.cr
ccacctcagtggtacttaatgc

SEQ ID NO: 469 - DNA, 20 bp, Artificial Sequence
  N.4.cr
cctcagtggtacttaatgct

SEQ ID NO: 470 - DNA, 21 bp, Artificial Sequence
  N.5.cr
cttaatgctgatatgctcaca

SEQ ID NO: 471 - DNA, 23 bp, Artificial Sequence
  big.S.1
tttgctttataggagaaatggga

SEQ ID NO: 472 - DNA, 21 bp, Artificial Sequence
  big.S.2
ctttataggagaaatgggaca

SEQ ID NO: 473 - DNA, 24 bp, Artificial Sequence
  big.S.3
ttataggagaaatgggacaacttg

SEQ ID NO: 474 - DNA, 21 bp, Artificial Sequence
  big.S.4
gagaaatgggacaacttgtcc

SEQ ID NO: 475 - DNA, 21 bp, Artificial Sequence
  big.S.5
aaatgggacaacttgtccatc

SEQ ID NO: 476 - DNA, 21 bp, Artificial Sequence
  big.S.1.cr
gatggacaagttgtcccattt

SEQ ID NO: 477 - DNA, 21 bp, Artificial Sequence
  big.S.2.cr
gacaagttgtcccatttctcc

SEQ ID NO: 478 - DNA, 22 bp, Artificial Sequence
  big.S.3.cr
aagttgtcccatttctcctata

SEQ ID NO: 479 - DNA, 23 bp, Artificial Sequence
  big.S.4.cr
tgtcccatttctcctataaagca

SEQ ID NO: 480 - DNA, 23 bp, Artificial Sequence
  big.S.5.cr
cccatttctcctataaagcaaaa

SEQ ID NO: 481 - DNA, 21 bp, Artificial Sequence
  little.s.1
tgctttataggagaaacggga

SEQ ID NO: 482 - DNA, 20 bp, Artificial Sequence
  little.s.2
tttataggagaaacgggaca

SEQ ID NO: 483 - DNA, 19 bp, Artificial Sequence
  little.s.3
ggagaaacgggacaacttg

SEQ ID NO: 484 - DNA, 20 bp, Artificial Sequence
  little.s.4
gagaaacgggacaacttgtc

SEQ ID NO: 485 - DNA, 20 bp, Artificial Sequence
  little.s.5
aaacgggacaacttgtccat

SEQ ID NO: 486 - DNA, 19 bp, Artificial Sequence
  little.s.1.cr
tggacaagttgtcccgttt

SEQ ID NO: 487 - DNA, 20 bp, Artificial Sequence
  little.s.2.cr
acaagttgtcccgtttctcc

SEQ ID NO: 488 - DNA, 21 bp, Artificial Sequence
  little.s.3.cr
agttgtcccgtttctcctata

SEQ ID NO: 489 - DNA, 22 bp, Artificial Sequence
  little.s.4.cr
tgtcccgtttctcctataaagc

SEQ ID NO: 490 - DNA, 20 bp, Artificial Sequence
  little.s.5.cr
cccgtttctcctataaagca

SEQ ID NO: 491 - DNA, 20 bp, Artificial Sequence
  U.pos.1
ttgctgctctctttagctcc

SEQ ID NO: 492 - DNA, 23 bp, Artificial Sequence
  U.pos.2
ctctctttagctcctgtagtgat

SEQ ID NO: 493 - DNA, 23 bp, Artificial Sequence
  U.pos.3
agctcctgtagtgataatactca

SEQ ID NO: 494 - DNA, 25 bp, Artificial Sequence
  U.pos.4
gtagtgataatactcattatttttg

SEQ ID NO: 495 - DNA, 23 bp, Artificial Sequence
  U.pos.5
taatactcattatttttggggtg

SEQ ID NO: 496 - DNA, 23 bp, Artificial Sequence
  U.pos.1.cr
caccccaaaaataatgagtatta

SEQ ID NO: 497 - DNA, 26 bp, Artificial Sequence
  U.pos.2.cr
caaaaataatgagtattatcactaca

SEQ ID NO: 498 - DNA, 24 bp, Artificial Sequence
  U.pos.3.cr
agtattatcactacaggagctaaa

SEQ ID NO: 499 - DNA, 21 bp, Artificial Sequence
  U.pos.4.cr
atcactacaggagctaaagag

SEQ ID NO: 500 - DNA, 20 bp, Artificial Sequence
  U.pos.5.cr
gagctaaagagagcagcaaa

SEQ ID NO: 501 - DNA, 22 bp, Artificial Sequence
  U.neg.1
ttttgctgctctctttatctcc

SEQ ID NO: 502 - DNA, 24 bp, Artificial Sequence
  U.neg.2
gctctctttatctcctgtagagat

SEQ ID NO: 503 - DNA, 24 bp, Artificial Sequence
  U.neg.3
tatctcctgtagagataacactca

SEQ ID NO: 504 - DNA, 24 bp, Artificial Sequence
  U.neg.4
gtagagataacactcattattttt

SEQ ID NO: 505 - DNA, 22 bp, Artificial Sequence
  U.neg.5
taacactcattatttttggggt

SEQ ID NO: 506 - DNA, 22 bp, Artificial Sequence
  U.neg.1.cr
accccaaaaataatgagtgtta

SEQ ID NO: 507 - DNA, 25 bp, Artificial Sequence
  U.neg.2.cr
aaaaataatgagtgttatctctaca

SEQ ID NO: 508 - DNA, 24 bp, Artificial Sequence
  U.neg.3.cr
agtgttatctctacaggagataaa

SEQ ID NO: 509 - DNA, 23 bp, Artificial Sequence
  U.neg.4.cr
atctctacaggagataaagagag

SEQ ID NO: 510 - DNA, 24 bp, Artificial Sequence
  U.neg.5.cr
gagataaagagagcagcaaaatta

SEQ ID NO: 511 - DNA, 19 bp, Artificial Sequence
  Rh.big.C.1
tgagccagttcccttctgg

SEQ ID NO: 512 - DNA, 18 bp, Artificial Sequence
  Rh.big.C.2
gagccagttcccttctgg

SEQ ID NO: 513 - DNA, 19 bp, Artificial Sequence
  Rh.big.C.3
ctgagccagttcccttctg

SEQ ID NO: 514 - DNA, 18 bp, Artificial Sequence
  Rh.big.C.4
ccttctgggaaggtggtc

SEQ ID NO: 515 - DNA, 19 bp, Artificial Sequence
  Rh.big.C.5
ccttctgggaaggtggtca

SEQ ID NO: 516 - DNA, 19 bp, Artificial Sequence
  Rh.big.C.1.cr
tgaccaccttcccagaagg

SEQ ID NO: 517 - DNA, 18 bp, Artificial Sequence
  Rh.big.C.2.cr
gaccaccttcccagaagg

SEQ ID NO: 518 - DNA, 18 bp, Artificial Sequence
  Rh.big.C.3.cr
ccagaagggaactggctc

SEQ ID NO: 519 - DNA, 19 bp, Artificial Sequence
  Rh.big.C.4.cr
ccagaagggaactggctca

SEQ ID NO: 520 - DNA, 19 bp, Artificial Sequence
  Rh.big.C.5.cr
cagaagggaactggctcag

SEQ ID NO: 521 - DNA, 18 bp, Artificial Sequence
  Rh.little.c.1
gagccagttccctcctgg

SEQ ID NO: 522 - DNA, 17 bp, Artificial Sequence
  Rh.little.c.2
agccagttccctcctgg

SEQ ID NO: 523 - DNA, 18 bp, Artificial Sequence
  Rh.little.c.3
tgagccagttccctcctg

SEQ ID NO: 524 - DNA, 17 bp, Artificial Sequence
  Rh.little.c.4
cctcctgggaaggtggt

SEQ ID NO: 525 - DNA, 18 bp, Artificial Sequence
  Rh.little.c.5
cctcctgggaaggtggtc

SEQ ID NO: 526 - DNA, 18 bp, Artificial Sequence
  Rh.little.c.1.cr
gaccaccttcccaggagg

SEQ ID NO: 527 - DNA, 17 bp, Artificial Sequence
  Rh.little.c.2.cr
accaccttcccaggagg

SEQ ID NO: 528 - DNA, 17 bp, Artificial Sequence
  Rh.little.c.3.cr
ccaggagggaactggct

SEQ ID NO: 529 - DNA, 18 bp, Artificial Sequence
  Rh.little.c.4.cr
ccaggagggaactggctc

SEQ ID NO: 530 - DNA, 18 bp, Artificial Sequence
  Rh.little.c.5.cr
caggagggaactggctca

SEQ ID NO: 531 - DNA, 20 bp, Artificial Sequence
  RhC.intron.2.1
agggtgccctttgtcacttc

SEQ ID NO: 532 - DNA, 20 bp, Artificial Sequence
  RhC.intron.2.2
gccctttgtcacttcccagt

SEQ ID NO: 533 - DNA, 20 bp, Artificial Sequence
  RhC.intron.2.3
cctttgtcacttcccagtgg

SEQ ID NO: 534 - DNA, 22 bp, Artificial Sequence
  RhC.intron.2.4
ttgtcacttcccagtggtacaa

SEQ ID NO: 535 - DNA, 22 bp, Artificial Sequence
  RhC.intron.2.5
tcacttcccagtggtacaatca

SEQ ID NO: 536 - DNA, 20 bp, Artificial Sequence
  RhC.intron.2.1.cr
gaagtgacaaagggcaccct

SEQ ID NO: 537 - DNA, 20 bp, Artificial Sequence
  RhC.intron.2.2.cr
actgggaagtgacaaagggc

SEQ ID NO: 538 - DNA, 20 bp, Artificial Sequence
  RhC.intron.2.3.cr
ccactgggaagtgacaaagg

SEQ ID NO: 539 - DNA, 22 bp, Artificial Sequence
  RhC.intron.2.4.cr
tgtaccactgggaagtgacaaa

SEQ ID NO: 540 - DNA, 22 bp, Artificial Sequence
  RhC.intron.2.5.cr
tgattgtaccactgggaagtga

SEQ ID NO: 541 - DNA, 21 bp, Artificial Sequence
  Rh.big.E.1
gccaagtgtcaactctcctct

SEQ ID NO: 542 - DNA, 22 bp, Artificial Sequence
  Rh.big.E.2
caagtgtcaactctcctctgct

SEQ ID NO: 543 - DNA, 22 bp, Artificial Sequence
  Rh.big.E.3
gtgtcaactctcctctgctgag

SEQ ID NO: 544 - DNA, 23 bp, Artificial Sequence
  Rh.big.E.4
caactctcctctgctgagaagtc

SEQ ID NO: 545 - DNA, 20 bp, Artificial Sequence
  Rh.big.E.5
tctcctctgctgagaagtcc

SEQ ID NO: 546 - DNA, 21 bp, Artificial Sequence
  Rh.big.E.1.cr
ggacttctcagcagaggagag

SEQ ID NO: 547 - DNA, 22 bp, Artificial Sequence
  Rh.big.E.2.cr
cttctcagcagaggagagttga

SEQ ID NO: 548 - DNA, 22 bp, Artificial Sequence
  Rh.big.E.3.cr
ctcagcagaggagagttgacac

SEQ ID NO: 549 - DNA, 21 bp, Artificial Sequence
  Rh.big.E.4.cr
gcagaggagagttgacacttg

SEQ ID NO: 550 - DNA, 20 bp, Artificial Sequence
  Rh.big.E.5.cr
gaggagagttgacacttggc

SEQ ID NO: 551 - DNA, 21 bp, Artificial Sequence
  Rh.little.e.1
gccaagtgtcaactctgctct

SEQ ID NO: 552 - DNA, 22 bp, Artificial Sequence
  Rh.little.e.2
caagtgtcaactctgctctgct

SEQ ID NO: 553 - DNA, 21 bp, Artificial Sequence
  Rh.little.e.3
tgtcaactctgctctgctgag

SEQ ID NO: 554 - DNA, 21 bp, Artificial Sequence
  Rh.little.e.4
caactctgctctgctgagaag

SEQ ID NO: 555 - DNA, 20 bp, Artificial Sequence
  Rh.little.e.5
tctgctctgctgagaagtcc

SEQ ID NO: 556 - DNA, 21 bp, Artificial Sequence
  Rh.little.e.1.cr
ggacttctcagcagagcagag

SEQ ID NO: 557 - DNA, 21 bp, Artificial Sequence
  Rh.little.e.2.cr
ttctcagcagagcagagttga

SEQ ID NO: 558 - DNA, 21 bp, Artificial Sequence
  Rh.little.e.3.cr
tcagcagagcagagttgacac

SEQ ID NO: 559 - DNA, 21 bp, Artificial Sequence
  Rh.little.e.4.cr
gcagagcagagttgacacttg

SEQ ID NO: 560 - DNA, 20 bp, Artificial Sequence
  Rh.little.e.5.cr
gagcagagttgacacttggc

SEQ ID NO: 561 - DNA, 19 bp, Artificial Sequence
  RhD 1
ttccccacagctccatcat

SEQ ID NO: 562 - DNA, 20 bp, Artificial Sequence
  RhD 2
acagctccatcatgggctac

SEQ ID NO: 563 - DNA, 21 bp, Artificial Sequence
  RhD 3
tccatcatgggctacaacttc

SEQ ID NO: 564 - DNA, 21 bp, Artificial Sequence
  RhD 4
tcatgggctacaacttcagct

SEQ ID NO: 565 - DNA, 21 bp, Artificial Sequence
  RhD 5
gggctacaacttcagcttgct

SEQ ID NO: 566 - DNA, 21 bp, Artificial Sequence
  RhD cr 1
agcaagctgaagttgtagccc

SEQ ID NO: 567 - DNA, 21 bp, Artificial Sequence
  RhD cr 2
agctgaagttgtagcccatga

SEQ ID NO: 568 - DNA, 20 bp, Artificial Sequence
  RhD cr 3
gaagttgtagcccatgatgg

SEQ ID NO: 569 - DNA, 17 bp, Artificial Sequence
  RhD cr 4
gcccatgatggagctgt

SEQ ID NO: 570 - DNA, 18 bp, Artificial Sequence
  RhD cr 5
tgatggagctgtggggaa

SEQ ID NO: 571 - DNA, 19 bp, Artificial Sequence
  r′s.T.1
ggaaggtcaacttggtgca

SEQ ID NO: 572 - DNA, 21 bp, Artificial Sequence
  r′s.T.2
ggaaggtcaacttggtgcagt

SEQ ID NO: 573 - DNA, 19 bp, Artificial Sequence
  r′s.T.3
caacttggtgcagttggtg

SEQ ID NO: 574 - DNA, 19 bp, Artificial Sequence
  r′s.T.4
acttggtgcagttggtggt

SEQ ID NO: 575 - DNA, 20 bp, Artificial Sequence
  r′s.T.5
ttggtgcagttggtggtgat

SEQ ID NO: 576 - DNA, 20 bp, Artificial Sequence
  r′s.T.1.cr
catcaccaccaactgcacca

SEQ ID NO: 577 - DNA, 20 bp, Artificial Sequence
  r′s.T.2.cr
caccaccaactgcaccaagt

SEQ ID NO: 578 - DNA, 20 bp, Artificial Sequence
  r′s.T.3.cr
accaactgcaccaagttgac

SEQ ID NO: 579 - DNA, 21 bp, Artificial Sequence
  r′s.T.4.cr
actgcaccaagttgaccttcc

SEQ ID NO: 580 - DNA, 19 bp, Artificial Sequence
  r′s.T.5.cr
tgcaccaagttgaccttcc

SEQ ID NO: 581 - DNA, 20 bp, Artificial Sequence
  Rh.DVI.1
atttcaaccctcttggcctt

SEQ ID NO: 582 - DNA, 20 bp, Artificial Sequence
  Rh.DVI.2
aaccctcttggcctttgttt

SEQ ID NO: 583 - DNA, 20 bp, Artificial Sequence
  Rh.DVI.3
tcttggcctttgtttccttg

SEQ ID NO: 584 - DNA, 20 bp, Artificial Sequence
  Rh.DVI.4
ggtatcagcttgagagctcg

SEQ ID NO: 585 - DNA, 20 bp, Artificial Sequence
  Rh.DVI.5
atcagcttgagagctcggag

SEQ ID NO: 586 - DNA, 20 bp, Artificial Sequence
  Rh.DVI.1.cr
aaggccaagagggttgaaat

SEQ ID NO: 587 - DNA, 20 bp, Artificial Sequence
  Rh.DVI.2.cr
aaacaaaggccaagagggtt

SEQ ID NO: 588 - DNA, 20 bp, Artificial Sequence
  Rh.DVI.3.cr
caaggaaacaaaggccaaga

SEQ ID NO: 589 - DNA, 20 bp, Artificial Sequence
  Rh.DVI.4.cr
cgagctctcaagctgatacc

SEQ ID NO: 590 - DNA, 20 bp, Artificial Sequence
  Rh.DVI.5.cr
ctccgagctctcaagctgat

SEQ ID NO: 591 - DNA, 21 bp, Artificial Sequence
  RhD Y 1
tttctttgcagacttaggtgc

SEQ ID NO: 592 - DNA, 21 bp, Artificial Sequence
  RhD Y 2
ctttgcagacttaggtgcaca

SEQ ID NO: 593 - DNA, 22 bp, Artificial Sequence
  RhD Y 3
tttgcagacttaggtgcacagt

SEQ ID NO: 594 - DNA, 19 bp, Artificial Sequence
  RhD Y 4
acttaggtgcacagtgcgg

SEQ ID NO: 595 - DNA, 19 bp, Artificial Sequence
  RhD Y 5
cttaggtgcacagtgcggt

SEQ ID NO: 596 - DNA, 19 bp, Artificial Sequence
  RhD Y cr 1
caccgcactgtgcacctaa

SEQ ID NO: 597 - DNA, 20 bp, Artificial Sequence
  RhD Y cr 2
ccgcactgtgcacctaagtc

SEQ ID NO: 598 - DNA, 21 bp, Artificial Sequence
  RhD Y cr 3
gcactgtgcacctaagtctgc

SEQ ID NO: 599 - DNA, 20 bp, Artificial Sequence
  RhD Y cr 4
tgcacctaagtctgcaaaga

SEQ ID NO: 600 - DNA, 25 bp, Artificial Sequence
  RhD Y cr 5
cacctaagtctgcaaagaaatagcg

SEQ ID NO: 601 - DNA, 24 bp, Artificial Sequence
  RhD non Y 1
ctatttctttgcagacttatgtgc

SEQ ID NO: 602 - DNA, 22 bp, Artificial Sequence
  RhD non Y 2
tctttgcagacttatgtgcaca

SEQ ID NO: 603 - DNA, 22 bp, Artificial Sequence
  RhD non Y 3
ttgcagacttatgtgcacagtg

SEQ ID NO: 604 - DNA, 20 bp, Artificial Sequence
  RhD non Y 4
acttatgtgcacagtgcggt

SEQ ID NO: 605 - DNA, 20 bp, Artificial Sequence
  RhD non Y 5
cttatgtgcacagtgcggtg

SEQ ID NO: 606 - DNA, 20 bp, Artificial Sequence
  RhD non Y cr 1
acaccgcactgtgcacataa

SEQ ID NO: 607 - DNA, 21 bp, Artificial Sequence
  RhD non Y cr 2
accgcactgtgcacataagtc

SEQ ID NO: 608 - DNA, 21 bp, Artificial Sequence
  RhD non Y cr 3
gcactgtgcacataagtctgc

SEQ ID NO: 609 - DNA, 21 bp, Artificial Sequence
  RhD non Y cr 4
tgtgcacataagtctgcaaag

SEQ ID NO: 610 - DNA, 25 bp, Artificial Sequence
  RhD non Y cr 5
cacataagtctgcaaagaaatagcg

SEQ ID NO: 611 - DNA, 17 bp, Artificial Sequence
  Yt.a.1
gcgggagacttccacgg

SEQ ID NO: 612 - DNA, 18 bp, Artificial Sequence
  Yt.a.2
gggagacttccacggcct

SEQ ID NO: 613 - DNA, 18 bp, Artificial Sequence
  Yt.a.3
gagacttccacggcctgc

SEQ ID NO: 614 - DNA, 17 bp, Artificial Sequence
  Yt.a.4
gacttccacggcctgca

SEQ ID NO: 615 - DNA, 18 bp, Artificial Sequence
  Yt.a.5
ttccacggcctgcaggta

SEQ ID NO: 616 - DNA, 18 bp, Artificial Sequence
  Yt.a.1 CR
tacctgcaggccgtggaa

SEQ ID NO: 617 - DNA, 17 bp, Artificial Sequence
  Yt.a.2 CR
tgcaggccgtggaagtc

SEQ ID NO: 618 - DNA, 18 bp, Artificial Sequence
  Yt.a.3 CR
gcaggccgtggaagtctc

SEQ ID NO: 619 - DNA, 18 bp, Artificial Sequence
  Yt.a.4 CR
aggccgtggaagtctccc

SEQ ID NO: 620 - DNA, 17 bp, Artificial Sequence
  Yt.a.5 CR
ccgtggaagtctcccgc

SEQ ID NO: 621 - DNA, 18 bp, Artificial Sequence
  Yt.b.1
gcgggagacttcaacggc

SEQ ID NO: 622 - DNA, 19 bp, Artificial Sequence
  Yt.b.2
gggagacttcaacggcctg

SEQ ID NO: 623 - DNA, 19 bp, Artificial Sequence
  Yt.b.3
gagacttcaacggcctgca

SEQ ID NO: 624 - DNA, 18 bp, Artificial Sequence
  Yt.b.4
gacttcaacggcctgcag

SEQ ID NO: 625 - DNA, 19 bp, Artificial Sequence
  Yt.b.5
ttcaacggcctgcaggtaa

SEQ ID NO: 626 - DNA, 19 bp, Artificial Sequence
  Yt.b.1 CR
ttacctgcaggccgttgaa

SEQ ID NO: 627 - DNA, 18 bp, Artificial Sequence
  Yt.b.2 CR
ctgcaggccgttgaagtc

SEQ ID NO: 628 - DNA, 19 bp, Artificial Sequence
  Yt.b.3 CR
tgcaggccgttgaagtctc

SEQ ID NO: 629 - DNA, 19 bp, Artificial Sequence
  Yt.b.4 CR
caggccgttgaagtctccc

SEQ ID NO: 630 - DNA, 18 bp, Artificial Sequence
  Yt.b.5 CR
gccgttgaagtctcccgc

SEQ ID NO: 631 - DNA, 20 bp, Artificial Sequence
  a3
cagaccataagcacaggcgt

SEQ ID NO: 632 - DNA, 20 bp, Artificial Sequence
  a9
gctcgtccacagtgcgttat

SEQ ID NO: 633 - DNA, 20 bp, Artificial Sequence
  a17
cggcgttcaagcaaaccgaa

SEQ ID NO: 634 - DNA, 20 bp, Artificial Sequence
  a23
gacatatagctccactcaga

SEQ ID NO: 635 - DNA, 20 bp, Artificial Sequence
  a27
tagggtactgatgagcactc

SEQ ID NO: 636 - DNA, 38 bp, Artificial Sequence
  Wr-left
gccgcgaattcactagtgggcttcaaggtgtccaactc

SEQ ID NO: 637 - DNA, 38 bp, Artificial Sequence
  Wr-right
ggccgcgggaattcgattaggatgaagaccagcagagc

SEQ ID NO: 638 - DNA, 38 bp, Artificial Sequence
  Yt-left
ggccgcgggaattcgattccttcgtgcctgtggtagat

SEQ ID NO: 639 - DNA, 38 bp, Artificial Sequence
  Yt-right
gccgcgaattcactagtgttctgggacttctgggaatg

SEQ ID NO: 640 - DNA, 40 bp, Artificial Sequence
  Lu-left
gccgcgaattcactagtgggacccagagagagagagactg

SEQ ID NO: 641 - DNA, 37 bp, Artificial Sequence
  Lu-right
ggccgcgggaattcgattgggagtccagctggtatgg

SEQ ID NO: 642 - DNA, 20 bp, Artificial Sequence
  CS05 complement
catgagctagaagtcaggac

SEQ ID NO: 643 - DNA, 23 bp, Artificial Sequence
  primer Rex6AD3
tgtctagtttcttaccggcaagt

SEQ ID NO: 644 - DNA, 21 bp, Artificial Sequence
  primer R1068
attgccggctccgacggtatc

SEQ ID NO: 645 - DNA, 18 bp, Artificial Sequence
  primer R348
ctgaacagtgtgatgacc

SEQ ID NO: 646 - DNA, 19 bp, Artificial Sequence
  primer
tgatgaccaccttcccagg

SEQ ID NO: 647 - DNA, 18 bp, Artificial Sequence
  primer R637
gccctcttcttgtggatg

SEQ ID NO: 648 - DNA, 20 bp, Artificial Sequence
  primer
cagggccaccaccatttgaa

SEQ ID NO: 649 - DNA, 21 bp, Artificial Sequence
  primer
gaacatgccacttcactccag

SEQ ID NO: 650 - DNA, 20 bp, Artificial Sequence
  primer MN-left
gagggaatttgtcttttgca

SEQ ID NO: 651 - DNA, 18 bp, Artificial Sequence
  primer MN-Right
agaggcaagaattcctcc